(19) United States
(12) Patent Application Publication
(10) Pub. No.: US 2024/0096446 A1
(43) Pub. Date: Mar. 21, 2024

(54) MOTIF-BASED IDENTIFICATION OF FRAMEWORK AND COMPLEMENTARITY-DETERMINING REGIONS IN ADAPTIVE IMMUNE RECEPTORS

(71) Applicant: 10X GENOMICS, INC., Pleasanton, CA (US)

(72) Inventors: David Benjamin JAFFE, Pleasanton, CA (US); Wyatt James MCDONNELL, Pleasanton, CA (US)

(73) Assignee: 10X GENOMICS, INC., Pleasanton, CA (US)

(21) Appl. No.: 18/454,338

(22) Filed: Aug. 23, 2023

Related U.S. Application Data
(63) Continuation of application No. PCT/US2022/015986, filed on Feb. 10, 2022.
(60) Provisional application No. 63/153,292, filed on Feb. 24, 2021.

(51) Int. Cl. G16B 20/30; G16B 30/00; G16B 40/00

(52) U.S. Cl. CPC G16B 20/30; G16B 30/00; G16B 40/00

(57) ABSTRACT

A method for identifying framework regions and complementarity-determining regions in an amino acid sequence. The amino acid sequence is received. A plurality of candidate start positions is identified within the amino acid sequence for a start position for a selected region of interest. A score is generated for each candidate start position of the plurality of candidate start positions via analysis of a motif window that begins at each candidate start position. The start position for the selected region of interest is identified based on a candidate start position of the plurality of candidate start positions having a highest score.

### CROSS-REFERENCE TO RELATED APPLICATIONS

This application claims priority to U.S. Provisional Patent Application No. 63/153,292, filed Feb. 24, 2021, which is incorporated by reference herein in its entirety.

### FIELD

This description is generally directed towards systems and methods for identifying regions of interest in adaptive immune receptors. More specifically, methods and systems are provided for identifying framework and complementarity-determining regions in adaptive immune receptors via evolutionarily conserved motifs.

### BACKGROUND

Immune cell receptors are comprised of several key structural elements. Such key structural elements include highly variable complementarity-determining regions (CDRs) and intervening framework regions (FWRs). Being able to identify the locations of these regions within an amino acid sequence may help researchers identify the locations of mutations at key structural positions within the amino acid sequence. Knowing the locations of these mutations with respect to FWRs and CDRs may also help place these mutations in helpful contexts. For example, researchers may want to know whether mutations are present or absent in regions that need to have a low count of mutations for the development of a receptor into a biotherapeutic molecule. Further, being able to identify the locations of FWRs and CDRs may enable researchers to mine large datasets for signatures of immune recognition of pathogens. Still further, being able to identify the locations of FWRs and CDRs may enable researchers to group receptors into putative specificities despite differences in the amino acid sequences of the receptors. However, the high variability of these FWRs and CDRs may make identifying and locating these regions difficult.

### SUMMARY

In one or more embodiments, a method is provided for identifying framework regions and complementarity-determining regions in an amino acid sequence. The method includes receiving the amino acid sequence. The method further includes identifying a plurality of candidate start positions within the amino acid sequence for a start position for a selected region of interest. The method includes generating a score for each candidate start position of the plurality of candidate start positions via analysis of a motif window that begins at each candidate start position. The method includes identifying the start position for the selected region of interest based on a candidate start position of the plurality of candidate start positions having a highest score.

In one or more embodiments, a method is provided for identifying framework regions and complementarity-determining regions in an amino acid sequence. The method includes receiving the amino acid sequence and a chain type for the amino acid sequence. A first start position for FWR1 within the amino acid sequence is identified using an FWR1 motif. A second start position for CDR1 within the amino acid sequence is identified using the chain type, the first start position, and a CDR1 motif. A third start position for FWR2 within the amino acid sequence is identified using the chain type and an FWR2 motif. A fourth position for CDR2 within the amino acid sequence is identified using the chain type and the third start position. A fifth position for CDR3 within the amino acid sequence is identified using a CDR3 motif. A sixth position for FWR3 within the amino acid sequence is identified using the fifth position for FWR2 and a FWR3 motif selected based on the chain type. A seventh position for FWR4 is identified based on the fifth position. One or more of the first start position, the second start position, the third start position, the fourth start position, the fifth start position, and the sixth start position are validated. A sequence output that identifies sequences for one or more of the framework regions and complementarity-determining regions determined to have valid start positions is generated.

In one or more embodiments, a system is provided for identifying framework regions and complementarity-determining regions in an amino acid sequence. The system comprises a data source and a processor. The data source is configured to obtain the amino acid sequence generated from a sample. The amino acid sequence is for a chain of an immune cell receptor that is associated with an individual immune cell in the sample. The processor is configured to receive the amino acid sequence from the data source. The processor is further configured to: identify a plurality of candidate start positions within the amino acid sequence for a start position for a selected region of interest; generate a score for each candidate start position of the plurality of candidate start positions via analysis of a motif window that begins at each candidate start position; and identify the start position for the selected region of interest based on a candidate start position of the plurality of candidate start positions having a highest score.

In one or more embodiments, a non-transitory computer-readable medium in which a program is stored is provided. The program is configured for causing a computer to perform a method for identifying framework regions and complementarity-determining regions in an amino acid sequence. This method includes receiving the amino acid sequence; identifying a plurality of candidate start positions within the amino acid sequence for a start position for a selected region of interest; generating a score for each candidate start position of the plurality of candidate start positions via analysis of a motif window that begins at each candidate start position; and identifying the start position for the selected region of interest based on a candidate start position of the plurality of candidate start positions having a highest score.

These and other aspects and implementations are discussed in detail herein. The foregoing information and the following detailed description include illustrative examples of various aspects and implementations and provide an overview or framework for understanding the nature and character of the claimed aspects and implementations. The drawings provide illustration and a further understanding of the various aspects and implementations and are incorporated in and constitute a part of this specification.

### BRIEF DESCRIPTION OF FIGURES

The accompanying drawings are not intended to be drawn to scale. Like reference numbers and designations in the various drawings indicate like elements. For purposes of clarity, not every component may be labeled in every drawing. In the drawings:

FIG. 1 is a schematic diagram of a workflow for single cell sequencing in accordance with various embodiments.

FIG. 2 is an illustration of a V(D)J structure in accordance with one or more embodiments.

FIG. 3 is a flowchart of a process for identifying framework regions and complementarity-determining regions in an amino acid sequence in accordance with one or more embodiments.

FIG. 4 is a flowchart of a process for locating FWRs and CDRs in an amino acid sequence in accordance with one or more embodiments.

FIG. 5 is a flowchart of a process for identifying a start position for a selected region of interest in accordance with one or more embodiments.

FIG. 6 is a flowchart of a process for generating a sequence output identifying one or more FWRs, one or more CDRs, or both in an amino acid sequence in accordance with various embodiments.

FIG. 7 is an illustration of a position weight matrix for FWR1 in accordance with various embodiments.

FIG. 8 is an illustration of a position weight matrix for CDR1 in accordance with various embodiments.

FIG. 9 is an illustration of a position weight matrix for FWR2 in accordance with various embodiments.

FIG. 10 is an illustration of a set of position weight matrices for CDR2 in accordance with various embodiments.

FIG. 11 is an illustration of a set of position weight matrices for FWR3 in accordance with various embodiments.

FIG. 12 is an illustration of a position weight matrix for CDR3 in accordance with various embodiments.

FIG. 13 is a block diagram that illustrates a computer system in accordance with various embodiments.

FIG. 14 is a schematic diagram showing an exemplary capture probe, in accordance with various embodiments.

FIG. 15 is a schematic illustrating a cleavable capture probe, wherein the cleaved capture probe can enter into a non-permeabilized cell and bind to analytes within the sample, in accordance with various embodiments.

FIG. 16 is a schematic diagram of an exemplary multiplexed spatially-barcoded feature, in accordance with various embodiments.

FIG. 17A is a schematic diagram illustrating an exemplary embodiment of a spatial methodology for generating immune cell data (e.g., sequence data for an antigen binding molecule (ABM), in accordance with various embodiments.

FIG. 17B is a schematic diagram illustrating an exemplary embodiment of a spatial methodology for generating immune cell data, in accordance with various embodiments.

FIG. 18 is a schematic diagram illustrating an exemplary analyte enrichment strategy following analyte capture on the array, in accordance with various embodiments.

FIG. 19 is a schematic diagram illustrating a sequencing strategy with a primer specific complementary to the sequencing flow cell attachment sequence (e.g., P5) and a custom sequencing primer complementary to a portion of the constant region of the analyte, in accordance with various embodiments.

FIG. 20 is a schematic diagram illustrating an exemplary nucleic acid library preparation method to remove a portion of an analyte sequence via double circularization of a member of a nucleic acid library, in accordance with various embodiments.

FIG. 21 is a schematic diagram illustrating another exemplary workflow for processing such double-stranded circularized nucleic acid product, in accordance with various embodiments.

FIG. 22 is a schematic diagram illustrating an exemplary nucleic acid library preparation method to remove all or a portion of a constant sequence of an analyte from a member of a nucleic acid library via circularization, in accordance with various embodiments.

FIG. 23 is a schematic diagram illustrating an exemplary nucleic acid library method to reverse the orientation of an analyte sequence in a member of a nucleic acid library, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present disclosure in any way.

### DETAILED DESCRIPTION

### I. Introduction

The following description of various embodiments is exemplary and explanatory only and is not to be construed as limiting or restrictive in any way. Other embodiments, features, objects, and advantages of the present disclosure will be apparent from the description, accompanying drawings, and claims.

It should be understood that the use of subheadings herein is for organizational purposes and should not be read to limit the application of any feature described under a particular subheading to the various embodiments herein. Each and every feature described herein is applicable and usable in all the various embodiments discussed herein. Further, all features described herein may be used in any contemplated combination, regardless of the specific example embodiments that are described. Still further, the exemplary description of a specific feature is used largely for informational purposes and is not in any way meant to limit the design, sub-features, and/or functionality of the specifically described feature.

Any publication mentioned herein is incorporated by reference herein in its entirety for the purpose of describing and disclosing devices, compositions, formulations, and methodologies which are described in the publication and which might be used in connection with the present disclosure.

The various embodiments described herein provide a way of identifying framework regions (FWRs) and complementarity-determining regions (CDRs) of immune cell receptors (e.g., B cell receptors and T cell receptors) within a given amino acid sequence for these immune cell receptors. The various embodiments described herein provide a way of identifying FWRs and CDRs within an amino acid sequence with a greater deal of accuracy as compared to at least some currently available methodologies for identifying these regions. For example, numbering the same amino sequence for an immune cell receptor using different currently available methodologies may result in different answers for the identification of the FWRs and CDRs. For example, one currently available numbering system might provide different sequences for CDR1 and CDR2 as compared to another currently available numbering system. These differences may be, at least in part, due to various mutations within the amino acid sequence (e.g., insertions, deletions).

Thus, the various embodiments described herein provide methods and systems for identifying the sequences for FWRs and CDRs in an accurate and reliable manner regardless of whether mutations (e.g., insertions, deletions) have occurred. The various embodiments described herein enable identification of the sequences for FWRs and CDRs despite the FWRs and CDRs of different amino acid sequences having different lengths. The various embodiments provide a standardized approach for precisely defining the sequences for FWRs and CDRs more quickly, simply, and efficiently than at least some currently available methodologies and systems. Being able to precisely define these FWR and CDR sequences in adaptive immune receptors is important to understanding immune response, including the binding between immune receptors and antigens.

### II. Definitions & Exemplary Context

As used herein, the terms “comprise,” “comprises,” “comprising,” “contain,” “contains,” “containing,” “have,” “having,” “include,” “includes,” and “including” and their variants are not intended to be limiting, are inclusive or open-ended, and do not exclude additional, unrecited additives, components, integers, elements, or method steps. For example, a process, method, system, composition, kit, or apparatus that comprises a list of features is not necessarily limited only to those features but may include other features not expressly listed or inherent to such process, method, system, composition, kit, or apparatus.

Unless otherwise required by context, singular terms shall include pluralities and plural terms shall include the singular.

Unless defined otherwise, all scientific and technical terms used herein with respect to the various embodiments described herein have the same meaning as commonly understood by those of ordinary skill in the art.

Generally, the nomenclatures, techniques, and laboratory procedures described herein (e.g., in connection with cell and tissue culture, and molecular biology, as well as protein, oligonucleotide, and polynucleotide chemistry and hybridization) are ones that are well-known and commonly used in the art. Standard techniques may be used, for example, for nucleic acid purification and preparation, chemical analysis, recombinant nucleic acid, and oligonucleotide synthesis. Enzymatic reactions and purification techniques may be performed as described herein, according to manufacturer's specifications, as commonly accomplished in the art, or a combination thereof. Various techniques and procedures described herein are generally performed according to conventional methods well-known in the art and as described in various general and more specific references that are cited and discussed throughout the instant specification. See, e.g., Joseph Sambrook, David W. Russell, Molecular Cloning: A Laboratory Manual (Cold Spring Harbor Laboratory Press, 3rd ed. 2001).

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The terms “a,” “an,” and “the,” as used herein, generally refers to singular and plural references unless the context clearly dictates otherwise. “A and/or B” is used herein to include all of the following alternatives: “A”, “B”, “A or B”, and “A and B”.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to +5%, or up to +1%.

A “nucleotide,” as used herein, comprises a nucleoside and a phosphate group. A “nucleoside,” as used herein, comprises a nucleobase and a five-carbon sugar (e.g., ribose, deoxyribose, or analogs thereof). When the nucleobase is bonded to ribose, the nucleoside may be referred to as a ribonucleoside. When the nucleobase is bonded to deoxyribose, the nucleoside may be referred to as a deoxyribonucleoside. A “nucleobase,” which may be also referred to as a “nitrogenous base,” can take the form of one of five types: adenine (A), guanine (G), thymine (T), uracil (U), and cytosine (C).

A “polynucleotide,” “nucleic acid,” or “oligonucleotide” refers to a linear polymer of nucleotides (or nucleosides joined by internucleosidic linkages). Generally, a polynucleotide comprises at least three nucleotides. Generally, an oligonucleotide is comprised of nucleotides that range in number from a few nucleotides (or monomeric units) to several hundreds of nucleotides (monomeric units). Whenever a polynucleotide such as an oligonucleotide is represented by a sequence of letters, such as “ATGCCTG,” it will be understood that the nucleotides are in 5′→3′ order or direction from left to right and that “A” denotes adenine, “C” cytosine, “G” denotes guanine, and “T” denotes thymine, unless otherwise noted. The letters A, C, G, and T may be used to refer to the nucleobases themselves, as described above, the nucleosides that include those nucleobases, or the nucleotides that include those bases, as is standard in the art.

Deoxyribonucleic acid (DNA) is a chain of nucleotides consisting of 4 types of nucleotides: adenine (A), thymine (T), cytosine (C), and guanine (G). Ribonucleic acid (RNA) is comprised of 4 types of nucleotides: A, C, G, and uracil (U). Certain pairs of nucleotides specifically bind to one another in a complementary fashion, which may be referred to as complementary base pairing. For example, C pairs with G and A pairs with T. In the case of RNA, however, A pairs with U. When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” “fragment sequence,” or “nucleic acid sequencing read” denotes any information or data that is indicative of the order of the nucleotide bases (e.g., A, C, G, T/U) in a molecule (e.g., whole genome, whole transcriptome, exome, oligonucleotide, polynucleotide, fragment, etc.) of DNA or RNA. It should be understood that the present disclosure contemplates that this sequence information may be obtained using any of the available varieties of techniques, platforms, or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic-based systems, etc., or a combination thereof.

The term “barcode” may refer to a label, or identifier, that conveys or is capable of conveying information (e.g., information about an analyte in a sample, a bead, a feature, a capture probe, and/or a nucleic acid barcode molecule). A barcode can be part of an analyte, a capture probe, a reporter oligonucleotide, an analyte capture agent, or nucleic acid barcode molecule, or independent of an analyte, a capture probe, a reporter oligonucleotide, an analyte capture agent, or nucleic acid barcode molecule. A barcode can be attached to an analyte, a capture probe, a reporter oligonucleotide, an analyte capture agent, or nucleic acid barcode molecule in a reversible or irreversible manner. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for or facilitates identification and/or quantification of individual sequencing-reads. In some embodiments, a barcode can be configured for use as a fluorescent barcode. For example, in some embodiments, a barcode can be configured for hybridization to fluorescently labeled oligonucleotide probes. Barcodes can be configured to spatially resolve molecular components found in biological samples, for example, at single-cell resolution (e.g., a barcode can be or can include a “spatial barcode”). In some embodiments, a barcode includes two or more sub-barcodes that together function as a single barcode. For example, a polynucleotide barcode can include two or more polynucleotide sequences (e.g., sub-barcodes). In some embodiments, the two or more sub-barcodes are separated by one or more non-barcode sequences. In some embodiments, the two or more sub-barcodes are not separated by non-barcode sequences.

In some embodiments, a barcode can include one or more unique molecular identifiers (UMIs). Generally, a unique molecular identifier is a contiguous nucleic acid segment or two or more non-contiguous nucleic acid segments that function as a label or identifier for a particular analyte, or for a nucleic acid barcode molecule that binds a particular analyte (e.g., mRNA) via the capture sequence.

The term “barcoded nucleic acid molecule” generally refers to a nucleic acid molecule that results from, for example, the processing of a nucleic acid barcode molecule (e.g., a capture probe comprising a spatial barcode sequence) with a nucleic acid sequence (e.g., nucleic acid sequence complementary to a nucleic acid primer sequence encompassed by the nucleic acid barcode molecule). The nucleic acid sequence may be a targeted sequence or a non-targeted sequence. For example, hybridization and reverse transcription of a nucleic acid molecule (e.g., a messenger RNA (mRNA) molecule) of a cell with a nucleic acid barcode molecule (e.g., a nucleic acid barcode molecule containing a barcode sequence and a nucleic acid primer sequence complementary to a nucleic acid sequence of the mRNA molecule) results in a barcoded nucleic acid molecule that has a sequence corresponding to the nucleic acid sequence of the mRNA and the barcode sequence (or a reverse complement thereof). A barcoded nucleic acid molecule may serve as a template, such as a template polynucleotide, that can be further processed (e.g., amplified) and sequenced to obtain the target nucleic acid sequence. For example, a barcoded nucleic acid molecule may be further processed (e.g., amplified) and sequenced to obtain the nucleic acid sequence of the mRNA.

In some embodiments, where nucleic acid barcode molecule comprises a single cell barcode sequence, the nucleic acid barcode molecule may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcode molecule, or both, can include a nucleic acid reaction, such as, in non-limiting examples, reverse transcription, nucleic acid extension, ligation, etc. For example, the nucleic acid molecule comprising the nucleic acid sequence may be subjected to reverse transcription and then be attached to the nucleic acid barcode molecule to generate the barcoded nucleic acid molecule, or the nucleic acid molecule comprising the nucleic acid sequence may be attached to the nucleic acid barcode molecule and subjected to a nucleic acid reaction (e.g., extension, ligation) to generate the barcoded nucleic acid molecule. The barcoded nucleic acid molecule may serve as a template, such as a template polynucleotide, that can be further processed (e.g., amplified) and sequenced to obtain the target nucleic acid sequence. For example, the barcoded nucleic acid molecule may be further processed (e.g., amplified) and sequenced to obtain the nucleic acid sequence of the nucleic acid molecule (e.g., mRNA).

The term “cell barcode,” as used herein, refers to a known nucleotide sequence that serves as a unique identifier for a single GEM droplet. Each barcode usually contains reads from a single cell.

The term “clonotype,” as used herein, refers to a set of adaptive immune cells that are clonal progeny of a fully recombined, unmutated common ancestor. T cell clonotypes are generally distinguished by the nucleotide sequence of the rearranged TCR, which does not undergo somatic hypermutation (SHM) in most vertebrate species. B cell clonotypes are commonly divergent from each other at the nucleotide level. For this reason, B cell clonotypes also frequently contain multiple exact subclonotypes.

The term “exact subclonotype,” as used herein, refers to a subset of cells within a clonotype that share identical immune receptor sequences at the nucleotide level, spanning the entirety of the V, D, and J genes and the V(D)J junction. Exact subclonotypes share the same V, D, J, and C gene annotations (e.g. cells that have identical V(D)J sequences but different C genes or isotypes are split into distinct exact subclonotypes).

The term “sample,” as used herein, generally refers to a biological sample of a subject. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swap. The sample may be a plasma or serum sample. The sample may be a cell-free or cell free sample. A cell-free sample may include extracellular polynucleotides. Extracellular polynucleotides may be isolated from a bodily sample that may be selected from a group consisting of blood, plasma, serum, urine, saliva, mucosal excretions, sputum, stool and tears.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses). The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

The term “primer,” as used herein generally refers to a strand of RNA or DNA that serves as a starting point for nucleic acid (e.g., DNA) synthesis. A primer may be used in a primer extension reaction, which may be a nucleic acid amplification reaction, such as, for example, polymerase chain reaction (PCR) or reverse transcription PCR (RT-PCR). The primer may have a sequence that is capable of coupling to a nucleic acid molecule. Such sequence may be complementary to the nucleic acid molecule, such as a poly-T sequence or a predetermined sequence, or a sequence that is otherwise capable of coupling (e.g., hybridizing) to the nucleic acid molecule, such as a universal primer.

As used herein, the term “cell” is used interchangeably with the term “biological cell.” Non-limiting examples of biological cells include eukaryotic cells, plant cells, animal cells, such as mammalian cells, reptilian cells, avian cells, fish cells or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells and the like. A mammalian cell can be, for example, from a human, mouse, rat, horse, goat, sheep, cow, primate or the like.

As used herein, a genome is the genetic material of a cell or organism, including animals, such as mammals, e.g., humans. In humans, the genome includes the total DNA, such as, for example, genes, noncoding DNA and mitochondrial DNA. The human genome typically contains 23 pairs of linear chromosomes: 22 pairs of autosomal chromosomes plus the sex-determining X and Y chromosomes. The 23 pairs of chromosomes include one copy from each parent. The DNA that makes up the chromosomes is referred to as chromosomal DNA and is present in the nucleus of human cells (nuclear DNA). Mitochondrial DNA is located in mitochondria as a circular chromosome, is inherited from only the female parent, and is often referred to as the mitochondrial genome as compared to the nuclear genome of DNA located in the nucleus.

The phrase “sequencing” refers to any technique known in the art that allows the identification of consecutive nucleotides of at least part of a nucleic acid. Non-limiting exemplary sequencing techniques include RNA-seq (also known as whole transcriptome sequencing), Illumina™ sequencing, direct sequencing, random shotgun sequencing, Sanger dideoxy termination sequencing, whole-genome sequencing, massively parallel signature sequencing (MPSS), sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, and any combination thereof.

In general, the methods and systems described herein accomplish sequencing of nucleic acid molecules including, but not limited to, DNA (e.g., genomic DNA), RNA (e.g., mRNA, including full-length mRNA transcripts, and small RNAs, such as miRNA, tRNA, and rRNA), and cDNA. In various embodiments, the methods and systems described herein accomplish genomic sequencing of nucleic acid molecules (e.g., DNA, RNA, and mRNA). In various embodiments, the methods and systems described herein accomplish genomic sequencing of immune cell receptor sequences (e.g., DNA, RNA, and mRNA). In various embodiments, the methods and systems described herein can accomplish transcriptome sequencing, e.g., whole transcriptome sequencing of mRNA encoding immune cell receptors. In some embodiments, the methods and systems described herein can also accomplish targeted genomic sequencing of nucleic acid molecules (e.g., DNA, RNA, and mRNA). In various embodiments, the methods and systems described herein accomplish single cell genomic sequencing, for example, single cell genomic sequencing of nucleic acid molecules (e.g., RNA and mRNA) encoding immune cell receptors of single cells, such as B cell receptors (BCRs) and T cell receptors (TCRs).

In various embodiments, the methods and systems described herein can include high-throughput sequencing technologies, e.g., high-throughput DNA and RNA sequencing technologies. In various embodiments, the methods and systems described herein can include high-throughput, higher accuracy short-read DNA and RNA sequencing technologies. In various embodiments, the methods and systems described herein can include long-read RNA sequencing, e.g., by sequencing cDNA transcripts in their entirety without assembly. In various embodiments, the methods and systems described herein can also, for example, segment long nucleic acid molecules into smaller fragments that can be sequenced using high-throughput, higher accuracy short-read sequencing technologies, and that segmentation is accomplished in a manner that allows the sequence information derived from the smaller fragments to retain the original long range molecular sequence context, i.e., allowing the attribution of shorter sequence reads to originating longer individual nucleic acid molecules. By attributing sequence reads to an originating longer nucleic acid molecule, one can gain significant characterization information for that longer nucleic acid sequence that one cannot generally obtain from short sequence reads alone. This long-range molecular context is not only preserved through a sequencing process but is also preserved through the targeted enrichment process used in targeted sequencing approaches.

In general, the methods and systems described herein are directed to single cell analysis (including single- and multi-modal analyses) of genomic sequencing of nucleic acids (e.g., RNA and mRNA) encoding immune cell receptors of single cells, such as B cell receptors (BCRs) and T cell receptors (TCRs). Single cell analysis, including single cell multi-modal analyses (e.g., single cell immune cell receptor sequencing combined with, for example, gene expression, protein expression, and/or antigen capture technologies), as well as processing and sequencing of nucleic acids, in accordance with the methods and systems described in the present application are described in further detail, for example, in U.S. Pat. Nos. 9,689,024; 9,701,998; 10,011,872; 10,221,442; 10,337,061; 10,550,429; 10,273,541; and U.S. Pat. Pub. 20180105808, which are all herein incorporated by reference in their entirety for all purposes and in particular for all written description, figures and working examples directed to processing nucleic acids and sequencing and other characterizations of genomic material.

The term “B cells”, also known as B lymphocytes, refer to a type of white blood cell of the small lymphocyte subtype. They function in the humoral immunity component of the adaptive immune system by expressing and/or secreting antibodies. Additionally, B cells present antigens (they are also classified as professional antigen-presenting cells (APCs)) and secrete cytokines. In mammals, B cells mature in the bone marrow, which is at the core of most bones. In birds, B cells mature in the bursa of Fabricius, an immune organ where they were first discovered by Chang and Glick, (B for bursa) and not from bone marrow as commonly believed. B cells, unlike the other two classes of lymphocytes, T cells and natural killer cells, have the ability to secrete antibodies. Further, B cells can recognize intact antigen and therefore do not require that these antigens be located on or bound to peptide Major Histocompatibility Complex (pMHC) or human leukocyte antigen (HLA) molecules. BCRs allow a B cell to bind to specific antigens, against which it will initiate an antibody response.

The term “T cell”, also known as T lymphocytes, refer to a type of an adaptive immune cell. T cells develops in the thymus gland, hence the name T cell, and play a central role in the immune response of the body. T cells can be distinguished from other lymphocytes by the presence of a T cell receptor (TCR) on the cell surface. These immune cells originate as precursor cells, derived from bone marrow, and then develop into several distinct types of T cells once they have migrated to the thymus gland. T cell differentiation continues even after they have left the thymus. T cells include, but are not limited to, helper T cells, cytotoxic T cells, memory T cells, regulatory T cells, and killer T cells. Helper T cells stimulate B cells to make antibodies and help killer cells develop. Based on the T cell receptor chain, T cells can also include T cells that express αβ TCR chains, T cells that express γδ TCR chains, as well as unique TCR co-expressors (i.e., hybrid αβ-γδ T cells) that co-express the αβ and γδ TCR chains.

T cells can also include engineered T cells that can attack specific cancer cells. A patient's T cells can be collected and genetically engineered to produce chimeric antigen receptors (CAR). These engineered T cells are called CAR T cells, which forms the basis of the developing technology called CAR-T therapy. These engineered CAR T cells are grown by the billions in the laboratory and then infused into a patient's body, where the cells are designed to multiply and recognize the cancer cells that express the specific protein. This technology, also called adoptive cell transfer is emerging as a potential next-generation immunotherapy treatment.

T cells, such as the killer T cells can directly kill cells that have already been infected by a foreign invader. T cells can also use cytokines as messenger molecules to send chemical instructions to the rest of the immune system to ramp up its response. Activating T cells against cancer cells is the basis behind checkpoint inhibitors, a relatively new class of immunotherapy drugs that have recently been approved to treat lung cancer, melanoma, and other difficult cancers. Cancer cells often evade patrolling T cells by sending signals that make them seem harmless. Checkpoint inhibitors disrupt those signals and prompt the T cells to attack the cancer cells.

The term “naïve”, as used herein, can refer to B-lymphocytes or T-lymphocytes that have not yet reacted with an epitope of an antigen or that have a cellular phenotype consistent with that of a lymphocyte that has not yet responded to antigen-specific activation after clonal licensing.

The term “Fab”, also referred to as an antigen-binding fragment, refers to the variable portions of an antibody molecule with a paratope that enables the binding of a given epitope of a cognate antigen. The amino acid and nucleotide sequences of the Fab portion of antibody molecules are hypervariable. This is in contrast to the “Fc” or crystallizable fragment, which is relatively constant and encodes the isotype for a given antibody; this region can also confer additional functional capacity through processes such as antibody-dependent complement deposition, cellular cytotoxicity, cellular trogocytosis, and cellular phagocytosis.

The phrase “clonal selection” refers to the selection and activation of specific B lymphocytes and T lymphocytes by the binding of epitopes to B cell receptors or T cell receptors with a corresponding fit and the subsequent elimination (negative selection) or licensing for clonal expansion (positive selection) of a B or T lymphocyte after binding of an antigenic determinant.

The phrase “clonal expansion” refers to the proliferation of B lymphocytes and T lymphocytes activated by clonal selection in order to produce a clonal population of daughter cells with the same antigen specificity and functional capacity. In the case of T lymphocytes this antigen specificity is exact at the nucleotide and protein level and in the case of B lymphocytes this antigen specificity can be exact at the nucleotide and protein level or mutated relative to the parent population by mutations at the nucleotide level (and by extension the protein level). This enables the body to have enough antigen-specific lymphocytes to mount an effective immune response.

The term “cytokines” refers to a wide variety of intercellular regulatory proteins produced by many different cells in the body, which ultimately control every aspect of body defense. Cytokines activate and deactivate phagocytes and immune defense cells, enhance or inhibit the functions of the different immune defense cells, and promote or inhibit a variety of nonspecific body defenses.

The phrase “T helper lymphocytes”, also referred to as helper cells, refer to a type of white blood cell that orchestrate the immune response and enhance the activities of the killer T-cells (those that destroy pathogens) and B cells (antibody and immunoglobulin producers).

The phrase “affinity maturation” refers to the gradual modification of the paratope and entire B cell receptor as a result of somatic hypermutation. B lymphocytes with higher affinity B cell receptors that can bind the epitope more tightly and, therefore, bind the epitope for a longer period are able to proliferate more and survive longer. These B cells can eventually differentiate into plasma cells, which secrete their antibodies and form the basis of serum-mediated immunity.

The phrase “somatic hypermutation” (SHM) refers to a cellular mechanism by which the adaptive immune system adapts to foreign elements confronting it (e.g. viruses, bacteria, biomolecules). A major component of the process of affinity maturation, SHM diversifies B cell receptors used to recognize foreign elements (antigens) and allows the immune system to adapt its response to new threats during the lifetime of an organism. Somatic hypermutation involves a programmed process of mutation predominantly affecting select framework and complementarity-determining regions of immunoglobulin genes. Unlike germline mutation, SHM operates at the level of an organism's individual immune cells. These mutations are not transmitted to the organism's offspring but are transmitted to daughter cells of individual B cell clones. Mistargeted somatic hypermutation is a likely mechanism in the development of B cell lymphomas and many other cancers. Somatic hypermutation can also lead to the acquisition of non-VDJ template DNA within B cell receptor sequences, such as LAIR1 insertions in malaria-specific neutralizing antibodies.

Somatic hypermutation is a distinct diversification mechanism from isotype switching (also called class switching). Mutations acquired during somatic hypermutation eventually lead to isotype switching, in which a B cell's antibody can be coupled to different functions by switching to a different Fc/constant region sequence. Isotype switching is an irreversible process, in that once a B cell has switched from a given constant region (e.g. IGHM) to a new constant region (e.g. IGHA1) it can no longer use the IgM constant region as the DNA encoding the IgM Fc is excised and removed during isotype switching.

The term “contig”, originating from the term “contiguous”, refers to a set of overlapping DNA segments that together represent a consensus region of DNA. In bottom-up sequencing projects, a contig refers to overlapping sequence data (reads); in top-down sequencing projects, contig refers to the overlapping clones that form a physical map of the genome that is used to guide sequencing and assembly. Contigs can thus refer both to overlapping DNA sequences and to overlapping physical segments (fragments) contained in clones depending on the context. Note that clone, in reference to overlapping clones, refers to individual bacteria or constructs (e.g. phagemids, cosmids, etc.) containing distinct insertions of genomes that were utilized in early efforts to map genomes

The phrase “heavy chain” refers to the large polypeptide subunit of an antibody (immunoglobulin). The first recombination event to occur is between one D and one J gene segment of the heavy chain locus. Any DNA between these two gene segments is deleted. This D-J recombination is followed by the joining of one V gene segment, from a region upstream of the newly formed DJ complex, forming a rearranged VDJ gene segment. All other gene segments between V and D segments are now deleted from the cell's genome. Primary transcript (unspliced RNA) is generated containing the VDJ region of the heavy chain and both the constant mu and delta chains (Cμ and Cδ) (i.e., the primary transcript contains the segments: V-D-J-Cμ-Cδ). The primary RNA is processed to add a polyadenylated (poly-A) tail after the C chain and to remove sequence between the VDJ segment and this constant gene segment. Translation of this mRNA leads to the production of the IgM heavy chain protein and the IgD heavy chain protein (its splice variant). Expression of the immunoglobulin heavy chain with one or more surrogate light chains constitutes the pre-B cell receptor that allows a B cell to undergo selection and maturation.

The phrase “light chain” refers to the small polypeptide subunit of an antibody (immunoglobulin). The kappa (κ) and lambda (λ) chains of the immunoglobulin light chain loci rearrange in a very similar way, except that the light chains lack a D segment. In other words, the first step of recombination for the light chains involves the joining of the V and J chains to give a VJ complex before the addition of the constant chain gene during primary transcription. Translation of the spliced mRNA for either the kappa or lambda chains results in formation of the Ig κ or Ig λ light chain protein. Assembly of the Ig heavy chain and one of the light chains results in the formation of membrane bound form of the immunoglobulin IgM that is expressed on the surface of the immature B cell. B cells may express up to two heavy chains and/or two light chains in respectively rare and uncommon instances through a phenomenon known as allelic inclusion. This phenomenon can only be directly observed using single-cell technologies, though it can be inferred with a degree of uncertainty using a combination of bulk sequencing technologies and probabilistic inference via an extension of the birthday paradox.

The phrase “complementarity-determining regions” (CDRs) refers to part of the variable chains in immunoglobulins (antibodies) and T cell receptors, generated by B cells and T cells respectively, where these molecules are particularly hypervariable. The antigen-binding site of most antibodies and T cell receptors is typically distributed across these CDRs, collectively forming a paratope. However, there are many documented examples of paratopes that enable antigen recognition that fall outside of the CDRs. As the most variable parts of the molecules, CDRs are crucial to the diversity of antigen specificities and immune cell receptor sequences generated by lymphocytes.

V(D)J recombination is a genetic recombination mechanism that occurs in developing lymphocytes during the early stages of T and B cell maturation. Through somatic recombination, this mechanism produces a highly diverse repertoire of antibodies/immunoglobulins and T cell receptors (TCRs) found in B cells and T cells, respectively. This process is a defining feature of the adaptive immune system and these receptors are defining features of adaptive immune cells.

V(D)J recombination occurs in the primary immune organs (bone marrow for B cells and thymus for T cells) and in a generally random fashion. The process leads to the rearranging of variable (V), joining (J), and in some cases, diversity (D) gene segments. As discussed above, the heavy chain possesses numerous V, D, and J gene segments, while the light chain possesses only V and J gene segments. The process ultimately results in novel amino acid sequences in the antigen-binding regions of immunoglobulins and TCRs that allow for the recognition of antigens from nearly all pathogens including, for example, bacteria, viruses, and parasites. Furthermore, the recognition can also be allergic in nature or may recognize host tissues and lead to autoimmunity.

Human antibody molecules, including B cell receptors (BCRs), include both heavy and light chains, each of which contains both constant (C) and variable (V) regions, and are genetically encoded on three loci. The first is the immunoglobulin heavy locus on chromosome 14, containing the gene segments for the immunoglobulin heavy chain. The second is the immunoglobulin kappa (κ) locus on chromosome 2, containing the gene segments for part of the immunoglobulin light chain. The third is the immunoglobulin lambda (λ) locus on chromosome 22, containing the gene segments for the remainder of the immunoglobulin light chain.

Each heavy or light chain contains multiple copies of different types of gene segments for the variable regions of the antibody proteins. For example, the human immunoglobulin heavy chain region contains two C gene segments (Cμ and Cδ), 44 V gene segments, 27 D gene segments and 6 J gene segments. The number of given segments present in any individual can vary, as these gene segments are carried in haplotypes; for this reason, inference of both the alleles present within an individual and the germline sequence of those alleles is an important step in correctly identifying B cell clonotypes. The light chains possess two C gene segments (Cλ and Cκ) and numerous V and J gene segments, but do not have D gene segments. DNA rearrangement causes one copy of each type of gene segment to mate with any given lymphocyte, generating a substantial antibody repertoire. Approximately 10¹⁴ combinations are possible, with 1.5×10² to 3×10³ potentially removed via self-reactivity.

Accordingly, each naïve B cell makes an antibody with a unique Fab site through a series of gene recombination steps, and later mutations, with the specific molecules of the given antibody attaching to the B cell's surface as a B cell receptor (BCR). These BCRs are then available to react with epitopes of an antigen.

The term “CDR3 (Complementarity-Determining Region 3), as used herein, refers to three complementarity-determining regions are the portions of the amino acid sequence of a T or B cell receptor which are predicted to bind to an antigen. The nucleotide region encoding CDR3 spans the V(D)J junction, making it more diverse than that of the other CDRs. This serves as a useful way to identify unique chains.

When the immune system encounters an antigen, epitopes of that antigen will be presented to many B lymphocytes. B lymphocytes must first rearrange a heavy chain that enables pre-B cell receptor ligand binding. B lymphocytes that bind multivalent self-targets after rearrangement of the light chain too strongly are eliminated and die or undergo a secondary recombination event, while B cells that do not bind self-targets too strongly are licensed to exit the bone marrow. The latter becomes available to respond to non-self antigens and to undergo clonal expansion. This process is known as clonal selection.

Cytokines produced by activated CD4 T helper lymphocytes enable those activated B lymphocytes (B cells) to rapidly proliferate to produce large clones of thousands of identical B cells. More specifically, when under threat (i.e., via bacteria, virus, etc.), the body releases white blood cells by the immune system. CD4 T lymphocytes help the response to a threat by triggering the maturation of other types of white blood cell. They produce special proteins, called cytokines, have plural functions, including the ability to summon all of the other immune cells to the area, and also the ability to cause nearby cells to differentiate (become specialized) into mature B cells and T cells.

Accordingly, while only a few B cells in the body may have an antibody molecule that can bind a particular epitope, eventually many thousands of cells are produced with the right specificity, allowing the body's immune system to act en masse. This is referred to as clonal expansion. Natural phenomena such as IgA deficiency and murine transgenic models have shown that there are multiple paths by which a B cell receptor can acquire novel antigen specificity even from a very limited repertoire through the processes of somatic hypermutation and affinity maturation.

As the B cells proliferate, they undergo affinity maturation as a result of somatic hypermutation. This allows the B cells to “fine-tune” the paratopes of the antibody to more effectively fit with the recognized epitopes. B cells with high affinity B cell receptors on their surface bind epitopes more tightly and for a longer period of time, which enables these cells to selectively proliferate. Over the course of this proliferation and expansion, these variant B cells differentiate into plasma cells that synthesize and secrete vast quantities of antibodies with Fab sites that fit the target epitopes very precisely.

The phrase “immune cell” refers to a cell that is part of the immune system and that helps the body fight infections and other diseases. Immune cells include innate immune cells (such as basophils, dendritic cells, neutrophils, etc.) that are the first line of body's defense and are deployed to help attack the invading foreign cells (e.g., cancer cells) and pathogens. The innate immune cells can quickly respond to foreign cells and pathogens to fight infection, battle a virus, or defend the body against bacteria. Immune cells can also include adaptive immune cells (such as lymphocytes including B cells and T cells). The adaptive immune cells can come into action when an invading foreign cells or pathogens slip through the first line of body's defense mechanism. The adaptive immune cells can take longer to develop, because their behaviors evolve from learned experiences, but they can tend to live longer than innate immune cells. Adaptive immune cells remember foreign invaders after their first encounter and fight them off the next time they enter the body. Both types of immune cells employ important natural defenses in helping the body fight foreign cells and pathogens for fighting infections and other diseases.

Accordingly, the immune cells of the disclosure can include, but are not limited to, neutrophils, eosinophils, basophils, mast cells, monocytes, macrophages, dendritic cells, natural killer cells, and lymphocytes (such as B cells and T cells). The immune cells of the disclosure can further include dual expresser cells or DE (such as unique dual-receptor-expressing lymphocytes that co-express functional B cell receptor (BCR) and T cell receptor (TCR)), cells with adaptive immune receptors that may diversify or may not diversify (including immune cells expressing a chimeric antigen receptor with a fixed nucleotide sequence or with the capacity to mutate), and TCR co-expressors (i.e., hybrid αβ-γδ T cells) that co-express both αβ and γδ TCR chains.

The phrase “immune cell receptor”, “immune receptor”, or “immunologic receptor” refers to a receptor or immune cell receptor sequence, usually on a cell membrane, which can recognize components of pathogenic microorganisms (e.g., components of bacterial cell wall, bacterial flagella or viral nucleic acids) and foreign cells (e.g., cancer cells), which are foreign and not found naturally on the host cells, or binds to a target molecule (for example, a cytokine), and causes a response in the immune system. The immune cell receptors of the immune system can include, but are not limited to, pattern recognition receptors (PRRs), Toll-like receptors (TLRs), killer activated and killer inhibitor receptors (KARs and KIRs), complement receptors, Fc receptors, B cell receptors, and T cell receptors.

The phrase “immune cell receptor sequences” of an immune cell receptor include both heavy and light chains, each of which contains both constant (C) and variable (V) regions. For example, B cell receptors (BCRs) or B cell receptor sequences (including human antibody molecules) comprise of immunoglobulin heavy and light chains, each of which contains both constant (C) and variable (V) regions. Each heavy or light chain not only contains multiple copies of different types of gene segments for the variable regions of the antibody proteins, but also contains constant regions. For example, the BCR or human immunoglobulin heavy chain contains two (2) constant (Constant mu (Cμ) and delta (Cδ)) gene segments and forty four (44) Variable (V) gene segments, plus twenty seven (27) Diversity (D) gene segments, and six (6) Joining (J) gene segments. The BCR light chains also possess two (2) constant gene segments ((Constant lambda (Cλ) and kappa (Cκ)) and numerous V and J gene segments, but do not have any D gene segments. DNA rearrangement (i.e., recombination events) in developing B cells can cause one copy of each type of gene segment to go in any given lymphocyte, generating an enormous antibody repertoire. Accordingly, the primary transcript (unspliced RNA) of a BCR heavy chain can be generated containing the VDJ region of the heavy chain and both the constant mu and delta chains (Cμ and Cδ), i.e., the heavy chain primary transcript can contain the segments: V-D-J-Cμ-Cδ). In case of the B cell receptor and human immunoglobulin light chain, the first step of recombination for the light chains involves the joining of the V and J chains to give a VJ complex before the addition of the constant chain gene during primary transcription. Translation of the spliced mRNA for either the constant κ (Cκ) or λ (Cλ) chains results in formation of the Ig κ or Igλ light chain protein.

In general, most T cell receptors (TCR) are composed of an alpha (a) chain and a beta (β) chain, each of which contains both constant (C) and variable (V) regions. Thus, the most common type of a T cell receptor is called an alpha-beta TCR because it is composed of two different chains, one α-chain and one beta β-chain. A less common type of TCR is the gamma-delta TCR, which contains a different set of chains, one gamma (γ) chain and one delta (δ) chain. The T cell receptor genes are similar to immunoglobulin genes for the BCR and undergo similar DNA rearrangement (i.e., recombination events) in developing T cells as for the B cells. For example, the alpha-beta TCR genes also contain multiple V, D, and J gene segments in their beta chains and V and J gene segments in their alpha chains, which are re-arranged during the development of the T cells to provide a cell with a unique T cell antigen receptor. Thus, the β-chain of the TCR can contain Vβ-Dβ-Jβ gene segments and constant domain (Cβ) genes resulting in a Vβ-Dβ-Jβ-Cβ sequence of the TCR β-chain. The re-arrangement of the alpha (α) chain of the TCR follows β chain rearrangement, and can include Vα-Jα gene segments and constant domain (Cα) genes resulting in a Vα-J α-Cα sequence of the TCR α-chain. Similar to the alpha-beta TCRs, the TCR-γ chain is produced by V-J recombination and can contain Vγ-Jγ gene segments and constant domain (Cγ) genes resulting in a Vγ-Jγ-Cγ sequence of the TCR γ-chain, while the TCR-δ chain is produced using V-D-J recombination, and can contain Vδ-Dδ-Jδ gene segments and constant domain (Cδ) genes resulting in a Vδ-Dδ-Jδ-Cδ sequence of the TCR δ-chain.

The phrase “immune cell receptor constant region sequence” or “immune receptor constant region sequence” refers to the constant region or constant region sequence of an immune cell receptor. For example, the immune cell receptor constant region sequence or immune receptor constant region sequence can include, but is not limited to, the constant mu (Cμ) and delta (Cδ) region genes and sequences of a BCR and immunoglobulin heavy chain, the constant lambda (Cλ) and kappa (Cκ) region genes and sequences of a BCR and immunoglobulin light chain, the alpha constant (Cα) region genes and sequences of a TCR α-chain sequence, the beta constant (Cβ) region genes and sequences of a TCR β-chain sequence, the gamma constant (Cγ) region genes and sequences of a TCR γ-chain sequence, and the delta constant (Cδ) region genes and sequences of a TCR α-chain sequence.

### III. Barcoding and Sequencing Methodologies

Various sequencing technologies or combinations of sequencing technologies may be used to generate data that provides sequence information for individual cells. Examples of such sequencing technologies include single cell sequencing technologies such as non-droplet and droplet-based microfluidic single cell sequencing (e.g., single cell genomic sequencing) technologies, array-based microwell- and nanowell-based single cell sequencing technologies (e.g., array-based microwell- and nanowell based single cell genomic sequencing), and in situ sequencing technologies. Other examples include spatial analysis methodologies, e.g., spatially indexed single cell technologies, which are described further herein.

Any known sequencing methods (e.g., including single cell sequencing methods and spatial analysis methodologies) can be used to provide immune cell data (e.g., single immune cell sequencing data or spatial data) in various embodiments. In various embodiments, with single cell sequencing methods, single cells can be separated into partitions such as droplets or wells, wherein each partition comprises a single cell with a known identifier like a barcode. The barcode can be attached to a support, for example, a bead, such as a solid bead or a gel bead.

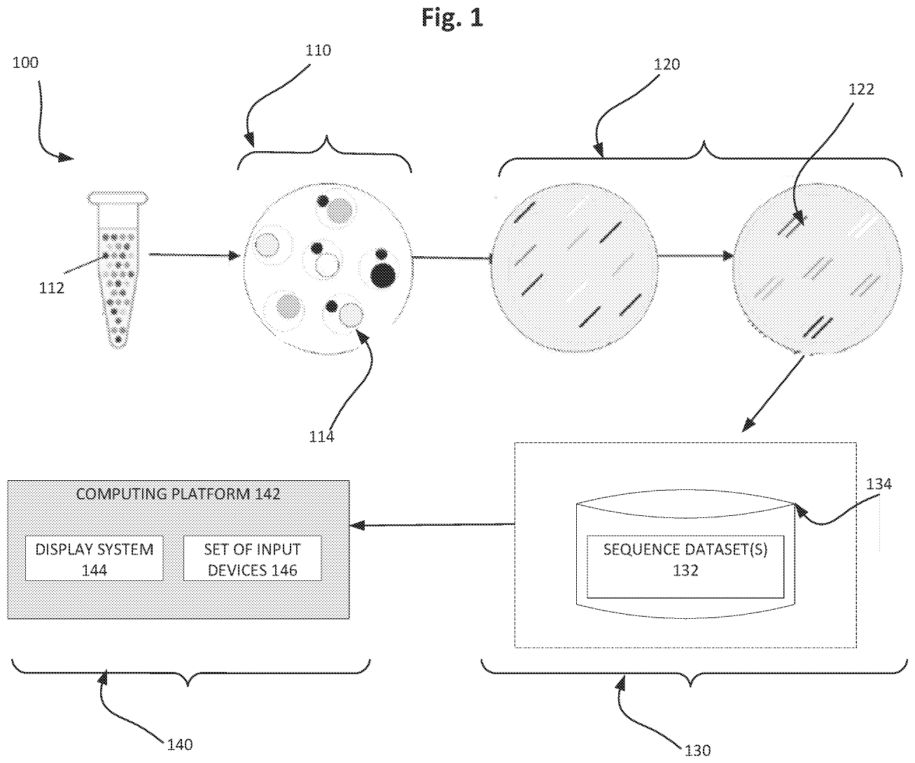

FIG. 1 is a schematic diagram of a workflow 100 for immune cell sequencing in accordance with various embodiments. Workflow 100 in FIG. 1 is an example of one manner in which immune cell sequencing (e.g., single cell or spatial analysis methodologies) may be implemented. It should be understood that in other example embodiments, workflow 100 may include one or more features in addition to or in place of the features described herein, one or more fewer features than described herein, or a combination thereof.

Workflow 100 may be used to generate immune cell data or sequence information for individual cells such as, for example, individual immune cells. For example, workflow 100 may be used to generate sequence information, antigen binding information, or a combination thereof, which may be used for identifying V(D)J information, clonotype information, antigen specificity, one or more other types of information, or a combination thereof in accordance with various embodiments. Workflow 100 includes sample preparation and processing 110, library construction 120, sequencing 130, and data analysis 140, which are further described below.

Sample preparation and processing 110, library construction 120, sequencing 130, and data analysis 140 may be performed using any number of or combination of the methodologies, systems, or concepts described herein and/or any number of or combination of methodologies, systems, or concepts described in U.S. Pat. Nos. 10,323,278, 10,550,429, 10,815,525, 10,725,027, 10,343,166, 10,583,440; U.S. Patent Application Publication Nos. 2018/0105808, 2018/0179590, 2019/0367969; U.S. Provisional Patent Application Nos. 63/135,493, 63/135,504, 63/135,514, and 63/135,519; and International Publication No. WO 2019/040637, each of which is incorporated herein by reference in its entirety.

Workflow 100 can include various combinations of features, whether it be more or less features than that illustrated in FIG. 1. As such, FIG. 1 simply illustrates one example of a possible workflow. Workflow 100 may include using, for example, single cell sequencing methodologies or spatial analysis methodologies. Accordingly, the single cell methodologies described below with respect to FIG. 1 are merely examples of how the workflow in FIG. 1 may be implemented and are not meant to limiting. Spatial analysis methodologies that may be included in workflow 100 in FIG. 1 are described further below.

### Sample Preparation and Processing

In one or more embodiments, sample preparation and processing 110 includes the preparation and processing of sample 112. Sample preparation and processing 110 may include partition-based approaches for processing single cells or their components or spatial array based methodologies, as described further herein.

In exemplary partition-based approaches, sample preparation and processing 110 includes, without limitation, the partitioning of sample 112 into wells (e.g., microwell arrays, nanowell arrays, etc.), droplets, or some other form of partition. Generally, in single cell sequencing, sample 112 is partitioned into a plurality of partitions 114 for a plurality of single cells.

Each “partition” of the partitions 114 of sample 112 is intended to capture a single cell. For example, a partition may generally include a single cell (or a lysate of a single cell) and a support. In one or more embodiments, this support includes (e.g., has bonded to it) a plurality of nucleic acid barcode molecules (e.g., polynucleotides such as, but not limited to, oligonucleotides, etc.) that uniquely identify the corresponding single cell. The nucleic acid barcode molecules of the plurality of nucleic acid barcode molecules share a common barcode sequence (or barcode). For example, in one or more embodiments, the support takes the form of a bead (e.g., a gel or hydrogel bead) and a plurality of oligonucleotides are provided on the bead that share a common barcode sequence. Thus, the bead is associated with a unique barcode sequence that is repeated in each of the plurality of oligonucleotides on the bead. In some embodiments, these beads are gel beads. A gel bead emulsion (GEM) contains a single cell, a single gel bead, and one or more reagents (e.g., lysis reagents, reverse transcriptase enzymes or reagents, etc.).

Sample preparation and processing 110 can result in the partitioning of sample 112 into partitions 114 with at least a subset of partitions 114 containing single cells that are comprised of analytes of interest (e.g., nucleic acid molecules such as, but not limited to, mRNA, etc.). In some embodiments, sample preparation and processing 110 further includes lysing the single cells within the partitions to release cellular components, including the analytes of interest (e.g., nucleic acid molecules or components), and barcoding the cellular components. The nucleic acid components released from a single cell within a particular partition are barcoded with the common barcode sequence that is associated with the support (e.g., bead) of the partition to thereby form barcoded nucleic acid molecules. These barcoded nucleic acid molecules may be sequenced to yield different types of information about the cells from which they originated.

In one or more embodiments, a partition that includes a single cell and a support associated with a unique barcode sequence may further include one or more reagents (e.g., lysis reagents including, for example, without limitation, bioactive reagents) to allow for processing of the single cell and the release of the cellular components of the single cell via lysis. For example, lysis may be used to release nucleic acid components such as, but not limited to, RNA (e.g., mRNA), DNA, or both. Further, lysis may also cause the release of the plurality of nucleic acid barcode molecules on the support such that these nucleic acid barcode molecules may anneal to and barcode the complements of the released nucleic acid components. In this manner, the various nucleic acid components that are released all include the common barcode sequence associated with the support within that partition.

For example, the support may be a bead that can be degraded (e.g., dissolved) via one or more reagents to release the nucleic acid barcode molecules (e.g., oligonucleotides that all share identical barcode sequences). The nucleic acid barcode molecules released from the support, as well as the nucleic acid components released from the single cell (e.g., mRNA) and reagents (e.g., reverse transcription (RT) reagents), within a partition are used to perform a nucleic acid extension reaction (e.g., reverse transcription of polyadenylated mRNA) to generate barcoded nucleic acid molecules (e.g., barcoded cDNA) within the partition. For example, all cDNA molecules that trace back to a same single cell within the partition will share an identical barcode sequence. In this manner, the barcoded nucleic acid molecules generated in a partition enable future sequencing to be mapped back to the original single cells from which the barcoded nucleic acid molecules originated.

Various protocols known in the art can be employed to generate samples such as sample 112 for use with one or more of the embodiments described herein. A sample (e.g., suspension) can be generated from any type of cells. For example, such cells may include eukaryotic cells (e.g., eukaryotic cells with a chromatin structure). Further, cells from fresh or cryopreserved cell lines (e.g., human cell lines, mouse cell lines, etc.), as well as more fragile primary cells, may be used. In one or more embodiments, the cells in a sample include, but are not limited to, immune cells (e.g., B cells, T cells), peripheral blood mononuclear cells (PBMCs), bone marrow mononuclear cells (BMMCs), lymphocytes, or a combination thereof. Still further, sample 112 may be formed by cells from a single donor or multiple donors.

### Library Construction

Library construction 120 includes the generation of a library 122, e.g., based on single cell or spatial analysis. In one or more embodiments, library 122 contains a plurality of DNA fragments. These DNA fragments may be utilized for sequencing, which occurs in sequencing 130. In one or more embodiments, barcoded cDNA molecules recovered from the partitions 114 formed and processed in sample preparation and processing 110 can be used as templates for multiplexed PCR to produce a single cell library. Library 122 may include molecules from one or more samples, molecules from samples from one or more donors, molecules from multiple libraries corresponding to one or more donors, or a combination thereof.

In one or more embodiments, library construction 120 includes library preparation. Library preparation may include, for example, adding one or more adapter sequences, optionally a sample index (SI) sequence, or a combination thereof to each of the recovered barcoded cDNA molecules in library construction 120. An SI sequence may include, for example, without limitation, one or more oligonucleotides (e.g., four oligonucleotides) that enable unique identification of the original sample, e.g., sample 112. In various embodiments, when analyzing the immune cell data (e.g., single cell sequencing data or spatial data) for a given sample, the sample index can be used to associate the data with the given sample.

### Sequencing

Sequencing 130 is performed to generate immune cell data such as one or more sequence datasets (or sequencing datasets) 132 based on the fully constructed library 122. Sequence dataset 132 may provide immune cell receptor information, e.g., on a single cell basis. In one or more embodiments, sequence dataset 132 may include a sequence (e.g., a codon sequence) for each molecule (e.g., barcoded cDNA molecule) included in library 122. Sequencing 130 may be performed using, for example, but is not limited to a next-generation sequencing (NGS) protocol. This NGS protocol may be implemented using any number of or combination of sequencing technologies and devices including, for example, without limitation, the sequencing technologies provided by Illumina®, Pacific Biosciences (PacBio®), Oxford Nanopore Technologies (ONT), etc.

Sequence dataset 132 is generated in a format that is compatible with data analysis 140 described below or that can be converted into a format compatible with data analysis 140. As one non-limiting example, sequence dataset 132 may be generated in a FASTQ format, which is a text-based format for storing biological sequences such as nucleotide sequences. In other embodiments, a different file format may be used for sequence dataset 132. In one or more embodiments, sequence dataset 132 is stored in data store 134. For example, data store 134 can be configured to store sequence dataset 132 for a single cell, including data for receptors (e.g., immune cell receptors), fragments thereof, or a combination thereof from single cells. Further, various software tools can be employed for processing and sending sequence dataset 132 as input into the downstream data analysis 140 portion of workflow 100. It is understood that various systems and methods with the embodiments herein are contemplated and can be employed to simultaneously analyze the inputted single cell sequencing data or spatial data for sequence analysis in accordance with various embodiments.

### Spatial Analysis Methodologies

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev D, dated October 2020), and/or the Visium Spatial Tissue Optimization Reagent Kits User Guide (e.g., Rev D, dated October 2020), both of which are available at the 10× Genomics Support Documentation website, and can be used herein in any combination. Further non-limiting aspects of spatial analysis methodologies and compositions are described herein.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

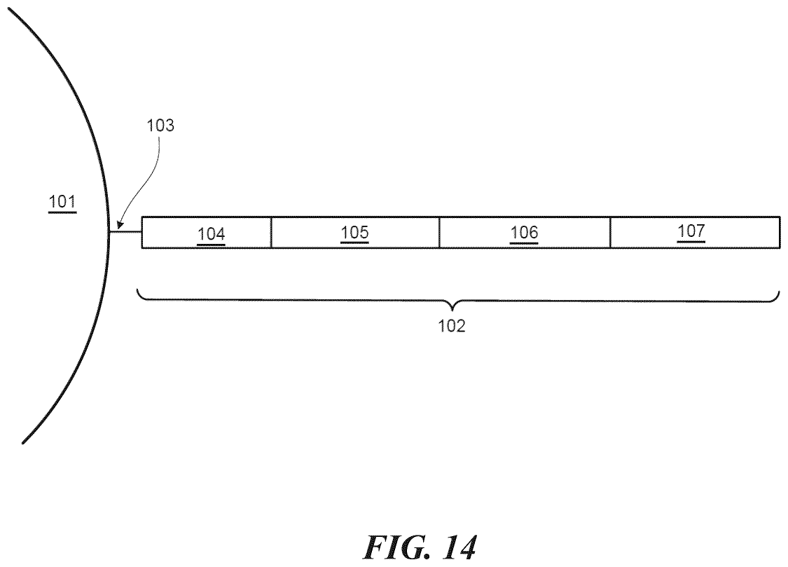

FIG. 14 is a schematic diagram showing an exemplary capture probe, as described herein. As shown in FIG. 14, the capture probe 102 is optionally coupled to a feature 101 by a cleavage domain 103, such as a disulfide linker. The capture probe can include a functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 14 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

Referring again to FIG. 14, in some embodiments, the spatial barcode 105 and functional sequences 104 are common to all of the probes attached to a given feature. In some embodiments, the UMI sequence 106 of a capture probe attached to a given feature is different from the UMI sequence of a different capture probe attached to the given feature.

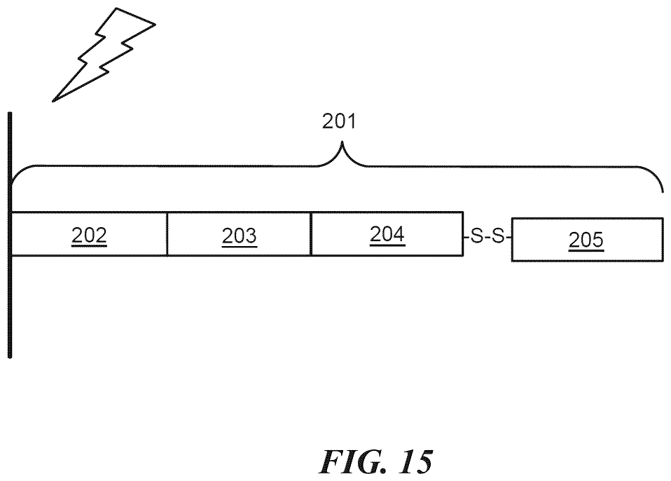

FIG. 15 is a schematic illustrating a cleavable capture probe, wherein the cleaved capture probe can enter into a non-permeabilized cell and bind to analytes within the sample. As shown in FIG. 15, the capture probe 201 contains a cleavage domain 202, a cell penetrating peptide 203, a reporter molecule 204, and a disulfide bond (—S—S—). 205 represents all other parts of a capture probe, for example a spatial barcode and a capture domain.

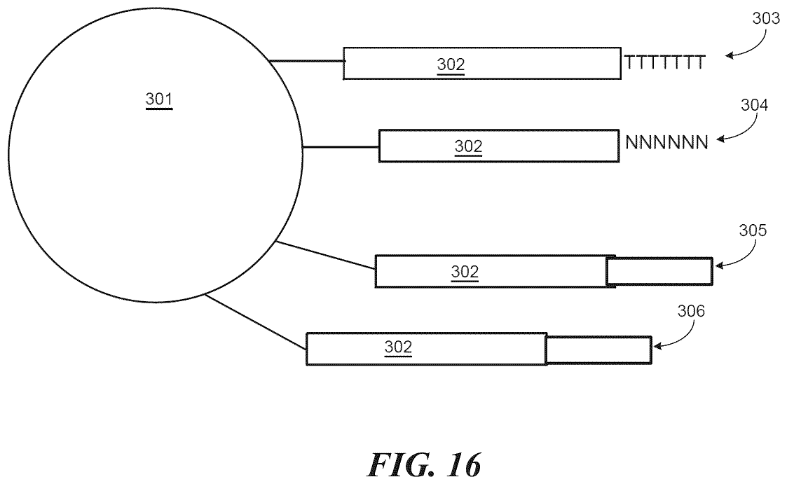

FIG. 16 is a schematic diagram of an exemplary multiplexed spatially-barcoded feature. In FIG. 16, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 16, capture-probe barcoded constructs can be tailored for analyses of any given analyte associated with a nucleic acid and capable of binding with such a construct. For example, the schemes shown in FIG. 16 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor) or antigen binding molecule (ABM).

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

For spatial array-based methods, a substrate may function as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample (e.g., a tissue sample) with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., a tissue sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Exemplary spatial methodologies for generating immune cell data (e.g., spatial datasets of at least one of immune cell receptors, antibodies, or fragments thereof from a tissue sample) are further described in WO2021247568 and WO2021247543, which are hereby incorporated by reference in their entirety. Such immune cell data may be obtained from tissue samples, e.g., tissue sections. The tissue section can be a fresh frozen tissue section, a fixed tissue section, or an FFPE tissue section. In some embodiments, the tissue sample is fixed and/or stained (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Tissue samples are also described in Section (I)(d) of WO 2020/176788 and/or U.S. Patent Application Publication No. 2020/0277663.

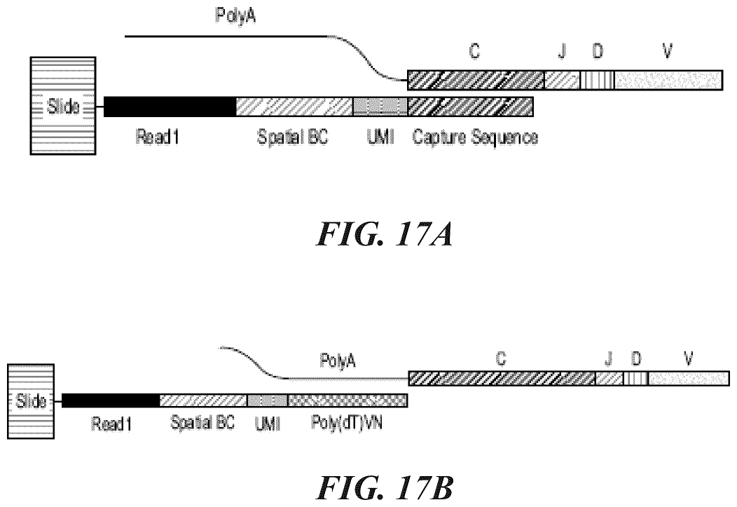

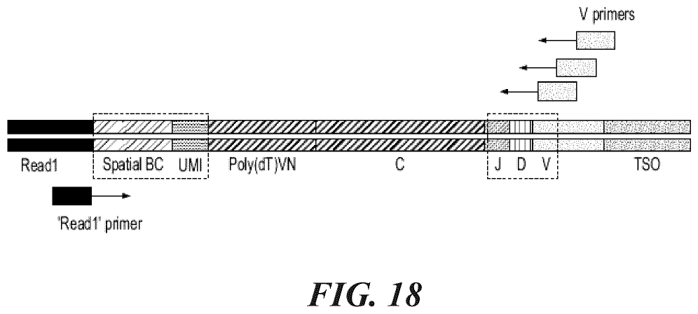

An exemplary embodiment of a spatial methodology for generating immune cell data (e.g., sequence data for an antigen binding molecule (ABM)) is depicted in FIG. 17A. An exemplary capture probe with a capture sequence that specifically binds to a nucleic acid sequence encoding a constant region of an ABM is depicted in FIG. 17A. In some embodiments, the ABM is selected from: a TCR alpha chain, a TCR beta chain, a TCR gamma chain, a TCR delta chain an immunoglobulin kappa light chain, an immunoglobulin lambda light chain, an immunoglobulin heavy chain. In some embodiments, the first capture sequence binds specifically to a nucleic acid sequence encoding a constant region of the T cell receptor alpha chain. In some embodiments, the first capture sequence binds specifically to a nucleic acid sequence encoding a constant region of the T cell receptor beta chain. In some embodiments, the first capture sequence binds specifically to a nucleic acid sequence encoding a constant region of the T cell receptor delta chain. In some embodiments, the first capture sequence binds specifically to a nucleic acid sequence encoding a constant region of the T cell receptor gamma chain. In some embodiments, the first capture sequence binds specifically to a nucleic acid sequence encoding a constant region of the immunoglobulin kappa light chain. In some embodiments, the first capture sequence binds specifically to a nucleic acid sequence encoding a constant region of the immunoglobulin lambda light chain. In some embodiments, the first capture sequence binds specifically to a nucleic acid sequence encoding a constant region of the immunoglobulin heavy chain.

Another exemplary embodiment of a spatial methodology for generating immune cell data is depicted in FIG. 17B. In such embodiments, the capture sequence sequence is a homopolymeric sequence, e.g., a polyT sequence. FIG. 17B shows an exemplary poly(A) capture with a poly(T) capture domain. A poly(T) capture domain can capture other analytes, including analytes encoding ABMs within the tissue sample.

In some embodiments, following capture of analytes by capture probes, capture probes can be extended, e.g., via reverse transcription. Second strand synthesis can generate double stranded cDNA products that are spatially barcoded. The double stranded cDNA products, which may comprise ABM encoding sequences and non-ABM related analytes, can be enriched for ABM encoding sequences.

An exemplary enrichment workflow may comprise amplifying the cDNA products (or amplicons thereof) with a first primer that specifically binds to a functional sequence of the first capture probe or reverse complement thereof and a second primer that binds to a nucleic acid sequence encoding a variable region of the ABM expressed by the ABM-expressing cell or reverse complement thereof. In some embodiments, the first primer and the second primer flank the spatial barcode of the first spatially barcoded polynucleotide or amplicon thereof. In some embodiments, the first primer and the second primer flank a J junction, a D junction, and/or a V junction.

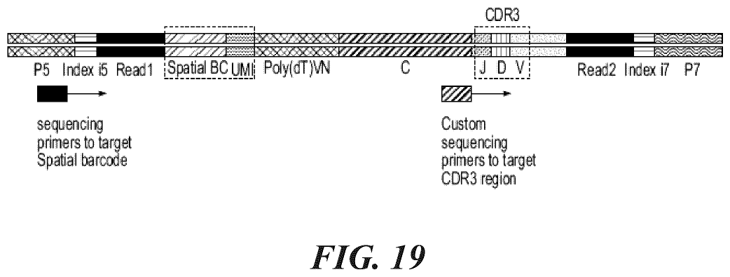

FIG. 18 shows an exemplary analyte enrichment strategy following analyte capture on the array. The portion of the immune cell analyte of interest includes the sequence of the V(D)J region, including CDR sequences. As described herein, a poly(T) capture probe captures an analyte encoding an ABM, an extended capture probe is generated by a reverse transcription reaction, and a second strand is generated. The resulting nucleic acid library can be enriched by the exemplary scheme shown in FIG. 18, where an amplification reaction including a Read 1 primer complementary to the Read 1 sequence of the capture probe and a primer complementary to a portion of the variable region of the immune cell analyte, can enrich the library via PCR. While FIG. 18 depicts a Read 1 primer, it is understood that a primer complementary to other functional sequences, such as other sequencing primer sequences, or sequencer specific flow cell attachment sequences, or portions of such functional sequences, may also be used. While FIG. 18 depicts a polyT capture sequence, it is understood that other capture sequences disclosed herein may be present in library members. The enriched library can be further enriched by nested primers complementary to a portion of the variable region internal (e.g., 5′) to the initial variable region primer for practicing nested PCR.

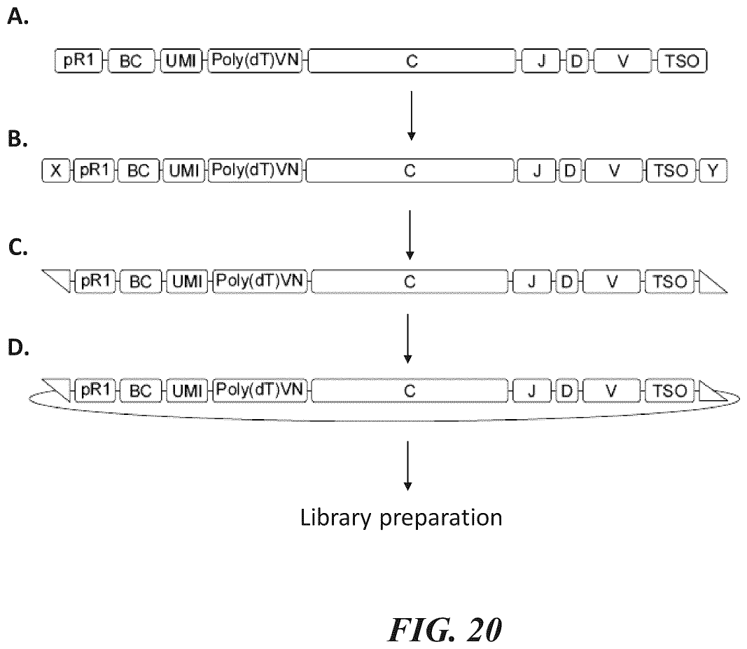

FIG. 19 shows a sequencing strategy with a primer specific complementary to the sequencing flow cell attachment sequence (e.g., P5) and a custom sequencing primer complementary to a portion of the constant region of the analyte. This sequencing strategy targets the constant region to obtain the sequence of the CDR regions, including CDR3, while concurrently or sequentially sequencing the spatial barcode (BC) and/or unique molecular identifier (UMI) of the capture probe. By capturing the sequence of a spatial barcode, UMI and a V(D)J region the receptor is not only determined, but its spatial location and abundance within a cell or tissue is also identified.

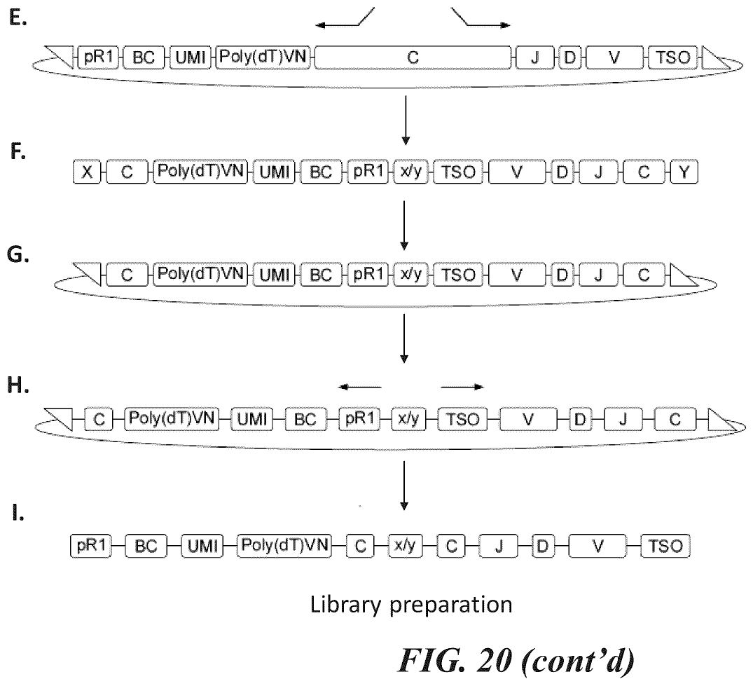

FIG. 20 shows an exemplary nucleic acid library preparation method to remove a portion of an analyte sequence via double circularization of a member of a nucleic acid library. Panel A shows an exemplary member of a nucleic acid library including, in a 5′ to 3′ direction, a first adaptor (e.g., primer sequence R1, pR1 (e.g., Read 1)), a barcode (e.g., a spatial barcode or a cell barcode), a unique molecular identifier (UMI), a capture domain (e.g., poly(T) VN sequence), a sequence complementary to an analyte (C, J, D and V), and a second adaptor (e.g., template switching oligonucleotide sequence (TSO)). For purposes of this example an analyte including a constant region (C) and V(D)J sequence are shown, however, the methods described herein can be equally applied to other analyte sequences in a nucleic acid library. Panel B shows the exemplary member of a nucleic acid library where additional sequences can be added to both the 3′ and 5′ ends of the nucleic acid member (shown as a X and Y) via a PCR reaction. The additional sequences added can include a recognition sequence for a restriction enzyme (e.g., restriction endonuclease). The restriction recognition sequence can be for a rare restriction enzyme. The exemplary member of the nucleic acid library shown in Panel B can be digested with a restriction enzyme to generate sticky ends shown in Panel C (shown as triangles) and can be intramolecularly circularized by ligation to generate the circularized member of the nucleic acid library shown in Panel D. The ligation can be performed with a DNA ligase. The ligase can be T4 ligase. A primer pair can be hybridized to a circularized nucleic acid member, where a first primer hybridizes to a 3′ portion of a sequence encoding the constant region (C) and includes a second restriction enzyme (e.g., restriction endonuclease) sequence that is non-complementary to the analyte sequence, and where a second primer hybridized to a 5′ portion of a sequence encoding the constant region (C), and where the second primer includes a second restriction enzyme sequence (Panel E). The first primer and the second primer can generate a linear amplification product (e.g., a first double-stranded nucleic acid product) as shown in Panel F, which includes the second restriction enzyme recognition sequences (shown as X and Y end sequences). The linear amplification product (Panel F) can be digested with a second restriction enzyme to generate sticky ends and can be intramolecularly ligated with a ligase (e.g., T4 DNA ligase) to generate a second double-stranded circularized nucleic acid product as shown in Panel G. The second double-stranded circularized nucleic product (Panel G) can be amplified with a third primer, pR1, substantially complementary to the first adaptor (e.g., Read 1) sequence and a fourth primer substantially complementary to the second adapter (e.g., TSO) as shown in Panel H to generate a version of the double-stranded member of the nucleic acid library lacking all, or a portion of, the sequence encoding the constant region (C) of the analyte (Panel I). The resulting double-stranded member of the nucleic acid library lacking all or a portion of the constant region can undergo library preparation methods, such as library preparation methods used in single-cell or spatial analyses. For example, the double-stranded member of the nucleic acid library lacking all, or a portion of, the sequence encoding the constant region of the analyte can be fragmented, followed by end repair, a-tailing, adaptor ligation, and/or additional amplification (e.g., PCR). The fragments can then be sequenced using, for example, paired-end sequencing using TruSeq Read 1 and TruSeq Read 2 as sequencing primer sites or any other sequencing method described herein. As such, sequences can be determined from regions more than about 1 kb away from the end of an analyte (e.g., 3′ end) and can link such a sequence to a barcode sequence (e.g., a spatial barcode, a cell barcode) in library preparation methods (e.g., sequencing preparation). For purposes of this example an analyte including a constant region (C) and V(D)J sequences are shown, however, the methods described herein can be equally applied to other analyte sequences in a nucleic acid library.

An exemplary member of a nucleic acid library can be prepared as shown in FIG. 20 to generate a first double-stranded circularized nucleic acid product shown in Panel D of FIG. 20 as previously described.

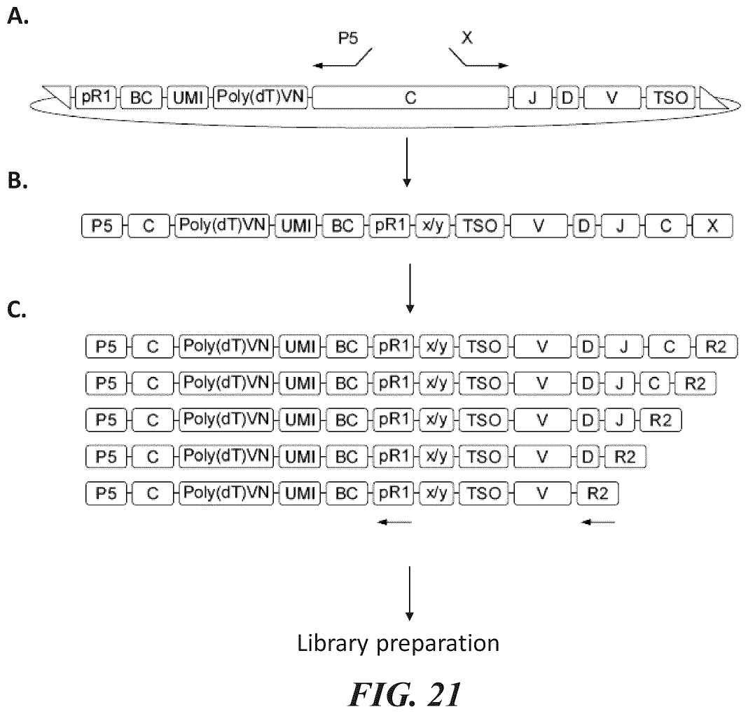

FIG. 21 depicts another exemplary workflow for processing such double-stranded circularized nucleic acid product. A primer pair can be contacted with the double-stranded circularized nucleic acid produce with a first primer that can hybridize to a sequence from a 3′ region of the sequence encoding the constant region of the analyte and a sequence including a first functional domain (e.g., P5). The second primer can hybridize to a sequence from a 5′ region of the sequence encoding the constant region of the analyte, and includes a sequence including a second functional domain (shown as “X”) as shown in Panel A. Amplification of the double-stranded circularized nucleic acid product results in a linear product as shown in Panel B, where all, or a portion of, the constant region (C) is removed. The first functional domain can include a sequencer specific flow cell attachment sequence (e.g., P5). The second functional domain can include an amplification domain such as a primer sequence to amplify the nucleic acid library prior to further sequencing preparation. The resulting double-stranded member of the nucleic acid library lacking all or a portion of the constant region can undergo library preparation methods, such as library preparation methods used in single-cell or spatial analyses. For example, the double-stranded member of the nucleic acid library lacking all, or a portion of, the sequence encoding the constant region of the analyte can be fragmented, followed by end repair, A-tailing, adaptor ligation, and/or amplification (e.g., PCR) (Panel C). The fragments can then be sequenced using, for example, paired-end sequencing using TruSeq Read 1 and TruSeq Read 2 as sequencing primer sites (Panel C, arrows), or any other sequencing method described herein. After library preparation methods described herein, a different sequencing primer for the first adaptor (e.g., Read 1) is used since the orientation of the first adaptor (e.g., Read 1) sequence will be reversed. Accordingly, sequences can be determined from regions more than about 1 kb away from the end of an analyte (e.g., 3′ end) and can link such a sequence to a barcode sequence (e.g., a spatial barcode, a cell barcode) in further library preparation methods (e.g., sequencing preparation). For purposes of this example an analyte including a constant region (C) and V(D)J sequence are shown, however, the methods described herein can be applied to other analyte sequences in a nucleic acid library as well.

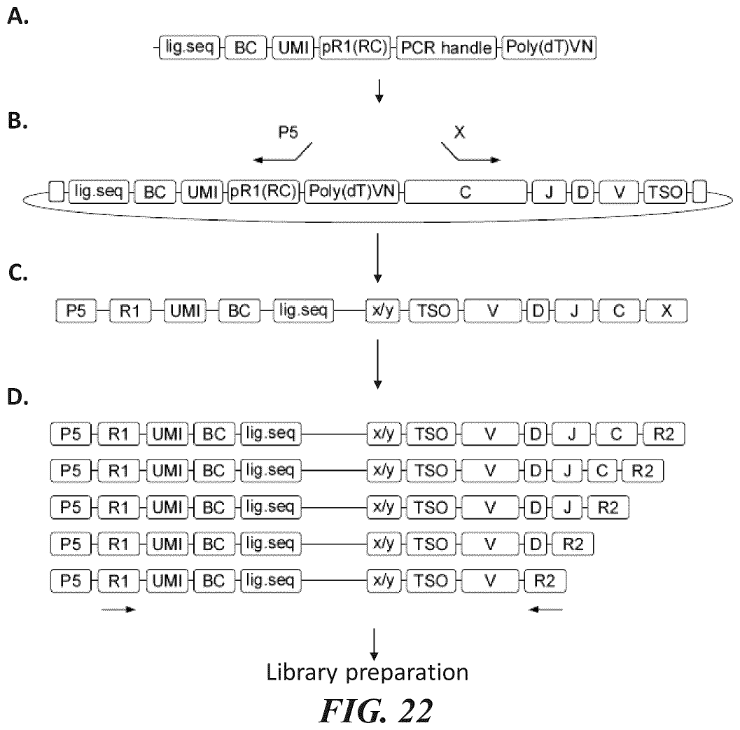

FIG. 22 shows an exemplary nucleic acid library preparation method to remove all or a portion of a constant sequence of an analyte from a member of a nucleic acid library via circularization. Panels A and B shows an exemplary member of a nucleic acid library including, in a 5′ to 3′ direction, a ligation sequence, a barcode sequence, a unique molecular identifier, a reverse complement of a first adaptor (e.g., primer sequence pR1 (e.g., Read 1)), a capture domain, a sequence complementary to the captured analyte sequence, and a second adapter (e.g., TSO sequence). The ends of the double-stranded nucleic acid can be ligated together via a ligation reaction where the ligation sequence splints the ligation to generate a circularized double-stranded nucleic acid as shown in Panel B. The circularized double-stranded nucleic acid can be amplified with a pair of primers to generate a linear nucleic acid product lacking all or a portion of the constant region of the analyte (Panels B and C). The first primer can include a sequence substantially complementary to the reverse complement of the first adaptor and a first functional domain. The first functional domain can be a sequencer specific flow cell attachment sequence (e.g., P5). The second primer can include a sequence substantially complementary to a sequence from a 5′ region of the sequence encoding the constant region of the analyte, and a second functional domain. The second functional domain can include an amplification domain such as a primer sequence to amplify the nucleic acid library prior to further sequencing preparation. The resulting double-stranded member of the nucleic acid library lacking all or a portion of the constant region can undergo library preparation methods, such as library preparation methods used in single-cell or spatial analyses. For example, the double-stranded member of the nucleic acid library lacking all, or a portion of, the sequence encoding the constant region of the analyte can be fragmented, followed by end repair, A-tailing, adaptor ligation, and/or amplification (e.g., PCR) (Panel C). The fragments can then be sequenced using, for example, paired-end sequencing using TruSeq Read 1 and TruSeq Read 2 as sequencing primer sites, or any other sequencing method described herein (Panel D). After library preparation methods (e.g., described herein), sequencing primers can be used since the orientation of Read 1 will be in the proper orientation for sequencing primer pR1. Accordingly, sequences can be determined from regions more than about 1 kb away from the end of an analyte (e.g., 3′ end) and can link such a sequence to a barcode sequence (e.g., a spatial barcode, a cell barcode) in further library preparation methods (e.g., sequencing preparation). For purposes of this example an analyte including a constant region (C) and V(D)J sequence are shown, however, the methods described herein can be applied to other analyte sequences in a nucleic acid library as well.

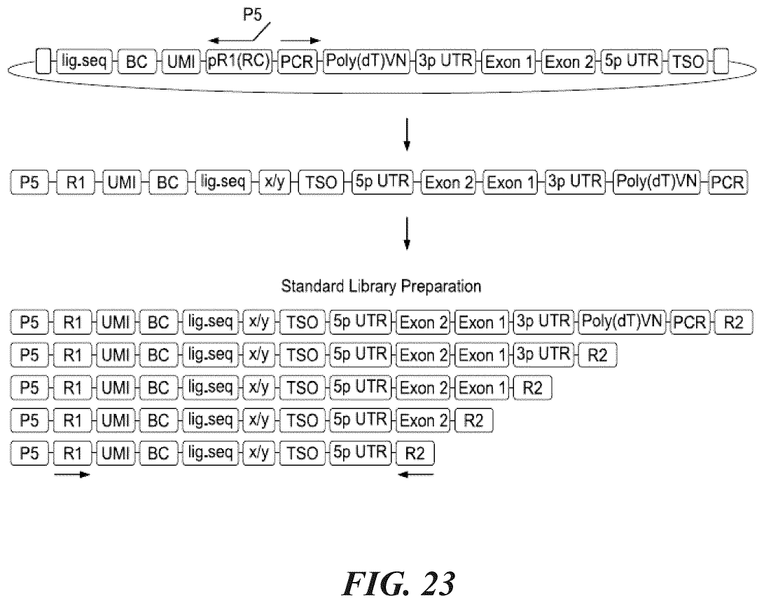

FIG. 23 shows an exemplary nucleic acid library method to reverse the orientation of an analyte sequence in a member of a nucleic acid library. Panel A shows an exemplary member of a nucleic acid library including, in a 5′ to 3′ direction, a ligation sequence, a barcode (e.g., a spatial barcode or a cell barcode), unique molecular identifier, a reverse complement of a first adaptor, an amplification domain, a capture domain, a sequence complementary to an analyte, and a second adapter. The ends of the double-stranded nucleic acid can be ligated together via a ligation reaction where the ligation sequence splints the ligation to generate a circularized double-stranded nucleic acid also shown in Panel A. The circularized double-stranded nucleic acid can be amplified to generate a linearized double-stranded nucleic acid product, where the orientation of the analyte is reversed such that the 5′ sequence (e.g., 5′ UTR) is brought in closer proximity to the barcode (e.g., a spatial barcode or a cell barcode) (Panel B). The first primer includes a sequence substantially complementary to the reverse complement of the first adaptor and a functional domain. The functional domain can be a sequencer specific flow cell attachment sequence (e.g., P5). The second primer includes a sequence substantially complementary to the amplification domain. The resulting double-stranded member of the nucleic acid library including a reversed analyte sequence (e.g., the 5′ end of the analyte sequence is brought in closer proximity to the barcode) can undergo library preparation methods, such as library preparation methods used in single-cell or spatial analyses. For example, the double-stranded member of the nucleic acid library lacking all, or a portion of, the sequence encoding the constant region of the analyte can be fragmented, followed by end repair, A-tailing, adaptor ligation, and/or amplification (e.g., PCR) (Panel C). The fragments can then be sequenced using, for example, paired-end sequencing using TruSeq Read 1 and TruSeq Read 2 as sequencing primer sites, or any other sequencing method described herein. Accordingly, sequences from the 5′ end of an analyte will be included in sequencing libraries (e.g., paired end sequencing libraries). Any type of analyte sequence in a nucleic acid library can be prepared by the methods described in this Example (e.g., reversed).

### Data Analysis

Data analysis 140 includes processing and analyzing sequence dataset 132. This analysis may be performed in any number of different ways to extract various pieces of information from sequence dataset 132. Various methods and systems may be employed to analyze sequence dataset 132 received as input in accordance with one or more embodiments described herein.

In one or more embodiments, data analysis 140 may be implemented using hardware, software, firmware, or a combination thereof. For example, data analysis 140 may be implemented using computing platform 142. Computing platform 142 may include a computer system, a cloud computing platform, some other type of computing platform, or a combination thereof. The computer system may include a single computer or multiple computers in communication with each other.

In one or more embodiments, computing platform 142 is communicatively coupled (e.g., via direct wired connection(s) or wireless connection(s)) to data store 134, display system 144, set of input devices 146, or a combination thereof. In one or more embodiments, display system 144, one or more input devices of set of input devices 146, or both are at least partially integrated within computing platform 142. In other embodiments, display system 144, one or more input devices of set of input devices 146, or a combination thereof may be separate from but in communication with computing platform 142. Computing platform 142 may receive, retrieve, or otherwise obtain sequence dataset 132 from data store 134. Display system 144 may be used to, for example, without limitation, visualize sequence dataset 132, information generated via data analysis 140, or both. Set of input devices 146 enable a user to provide user input for utilization during data analysis 140. Any combination or configuration of computing platform 142, data store 134, display system 144, or set of input devices 146 may be integrated into a system assembly (e.g., housed in a same housing and/or communicatively coupled via conventional device/component connection means).

The various embodiments, systems, and methods described herein include processing sequence dataset 132 via data analysis 140 to identify the locations of framework regions (FWRs) and complementarity-determining regions (CDRs) within a sequence. Knowing these locations may aid in the locating of mutations in the sequence with respect to region.

### IV. Motif-Based Identification of FWRs and CDRs in Adaptive Immune Receptors

As previously described, the various embodiments described herein provide methods and systems for identifying the sequences for FWRs and CDRs within a given amino acid sequence with a desired level of accuracy and precision regardless of whether mutations (e.g., insertions, deletions) have occurred. The various embodiments provide a standardized approach for defining these FWR and CDR sequences quickly, simply, and efficiently. The embodiments described herein enable identifying the sequences for FWRs and CDRs so that cells can be annotated into clonotypes to completion and more quickly as compared to existing systems and methodologies. For example, the embodiments described herein allow about 1.3 million cells to be annotated into clonotypes in about 90 minutes, while other existing systems would be unable to even complete such a process. The embodiments described herein are more robust than existing systems and methodologies such that using the embodiments described herein, indels within the FWRs and CDRs can be accurately identified. For example, HIV bnAb sequences containing indels may be correctly annotated using the embodiments described herein, where other existing software systems would mislabel the FWR and CDRs due to indels.

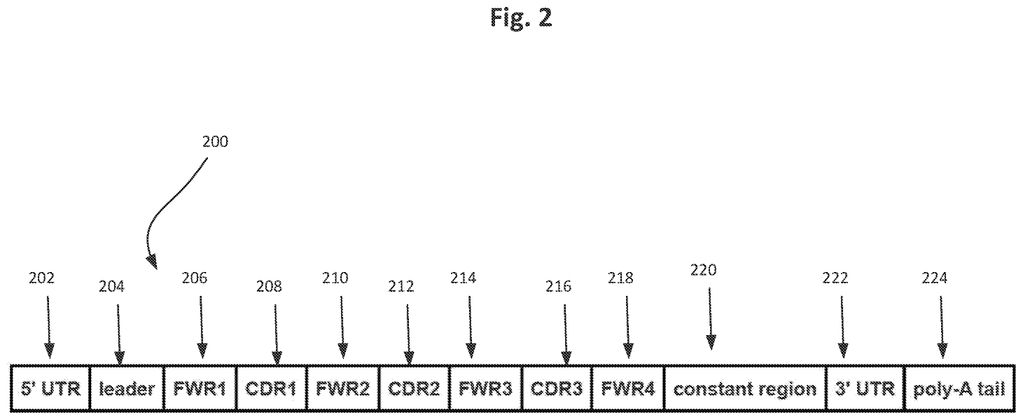

FIG. 2 is an illustration of a V(D)J structure 200 in accordance with one or more embodiments. V(D)J structure 200 is a structure of a V region of a chain of a protein complex such as, for example, an immune cell receptor. The chain may be a heavy chain, a kappa light chain, a lambda light chain, an alpha chain, or a beta chain. For example, a B cell receptor (e.g., antibody) is associated with heavy chains and light chains, while a T cell receptor is associated with an alpha chain and a beta chain.

V(D)J structure 200 includes the following components: 5′ untranslated region (5′ UTR) 202, leader region 204, framework region 1 (FWR1) 206, complementarity-determining region 1 (CDR1) 208, framework region 2 (FWR2) 210, complementarity-determining region 2 (CDR2) 212, framework region 3 (FWR3) 214, complementarity-determining region 3 (CDR3) 216, framework region 4 (FWR4) 218, constant region 220, 3′ untranslated region (3′ UTR) 222, and (polyadenylated) poly-A tail 224. The embodiments described herein provide methods and systems for defining the sequences for each of the FWR1 206, CDR1 208, FWR2 210, CDR2 212, FWR3 214, CDR3 216, and FWR4 218.

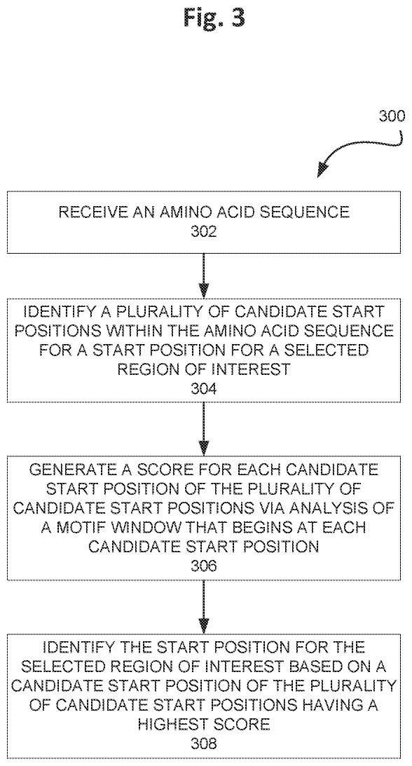

FIG. 3 is a flowchart of a process for identifying framework regions and complementarity-determining regions in an amino acid sequence in accordance with one or more embodiments. Process 300 is one example of a process that may be used to identify and locate the various FWRs and CDRs of an amino acid sequence for a chain. For example, process 300 is one example of a process that may be used to identify and locate the various FWRs and CDRs of V(D)J structure 200 in FIG. 2. In one or more embodiments, process 300 may be implemented using, for example, without limitation, computing platform 142 in FIG. 1.

Step 302 includes receiving an amino acid sequence. The amino acid sequence is a reference sequence that may be generated using, for example, workflow 100 in FIG. 1. In one or more embodiments, the amino acid sequence is included in a sequencing dataset generated for an immunoglobulin chain (e.g., heavy chain, lambda light chain, kappa light chain.). In one or more embodiments, the amino acid sequence is included in a sequencing dataset generated for T cell receptor chain (e.g., alpha chain, beta chain). In one or more embodiments, the amino acid sequence may be in an amino acid format, a nucleotide format, or an amino acid and nucleotide format. In one or more embodiments, each amino acid in the sequence corresponds to one position of the amino acid. These positions may be, for example, without limitation, numbered sequentially in the 5′ to 3′ direction.

Step 304 further includes identifying a plurality of candidate start positions within the amino acid sequence for a start position for a selected region of interest. The selected region of interest may be a FWR or a CDR. For example, the selected region of interest may be FWR1, FWR2, FWR3, or FWR4. Alternatively, the selected region of interest may be CDR1, CDR2, or CDR3. In one or more embodiments, the start position for a selected region of interest is the particular amino acid that demarcates the beginning of the selected region of interest. In one or more embodiments, a candidate start position is a position in the amino acid sequence that is identified as possibly being the start position for the selected region of interest. The plurality of candidate start positions may include any number of positions for evaluation such as, but not limited to, 5 positions, 8 positions, 9 positions, 13 positions, 23 positions, 34 positions, 40 positions, 50 positions, or some other number of positions selected from two positions up to and including the total number of positions in the amino acid sequence.

Step 306 includes generating a score for each candidate start position of the plurality of candidate start positions via analysis of a motif window that begins at each candidate start position. In one or more embodiments, the motif window is a subsequence of a selected number of positions. The motif window may also be referred to as an n-mer, where n is greater than one.

In one or more embodiments, generating the score for a corresponding candidate position includes evaluating a set of motif positions within the motif window that begins at the corresponding candidate position based on criteria for the set of amino acids at the set of motif positions. The score for the corresponding candidate start position is increased (e.g., points are added to the score) for any motif position in the set of motif positions that has an amino acid that matches the one or more criteria for that motif position. For example, without limitation, the one or more criteria for a particular motif position may include that the amino acid at that particular motif position matches one of a predetermined set of amino acids. If the motif position has an amino acid that matches an amino acid in the predetermined set of amino acids, the score for the corresponding candidate start position is increased.

In one or more embodiments, the score is generated using a position-weight matrix (PWM). For example, the PWM is used to identify a weight (or number of points) that is to be applied (e.g., added) to the score for each motif position of the set of motif positions within the motif window that meets the criteria for the amino acid at that position. Each candidate start position is given a score based on the PWM corresponding to the motif window that begins at that candidate start position. In one or more embodiments, each motif position of the set of motif positions is weighted differently from at least one other motif position of the set of motif positions.

In one or more embodiments, the motif window includes n motif positions. In various embodiments, the set of motif positions that are used for scoring within the motif window includes m positions, where m is less than or equal to n. A motif position in the set of motif positions is numbered with respect to the motif window, which is different from the numbering of the positions in the amino acid sequence. For example, without limitation, an 8^{th} motif position within the motif window may be the 8^{th} position with respect to a candidate start position at which the motif window begins, which may be 40^{th} overall position in the amino acid sequence.

Step 308 includes identifying the start position for the selected region of interest based on a candidate start position of the plurality of candidate start positions having a highest score. The highest scoring candidate start position is used to determine the start position of the selected region of interest. This determination can be made in various different ways. For example, in one or more embodiments, the start position is identified as the highest scoring candidate start position. In other embodiments, the start position is identified as some number of positions before or after the highest scoring candidate start position. In some embodiments, the start position is identified based on a formula that involves the highest scoring candidate start position.

Once the start position for the selected region of interest has been identified, this information may be used in various ways. In one or more embodiments, the start position is used to determine a start position for another region of interest in the amino acid sequence. In one or more embodiments, the start position is used to determine a plurality of candidate start positions for a start position for another region of interest in the amino acid sequence. Knowing the start position for the selected region of interest (e.g., a FWR or CDR) can enable, for example, without limitation, that selected region of interest to be “located” within the amino acid sequence. Knowing the location of the selected region of interest can, for example, without limitation, enable any mutations that are identified in the amino acid sequence to be identified as being located within or outside of the selected region of interest.

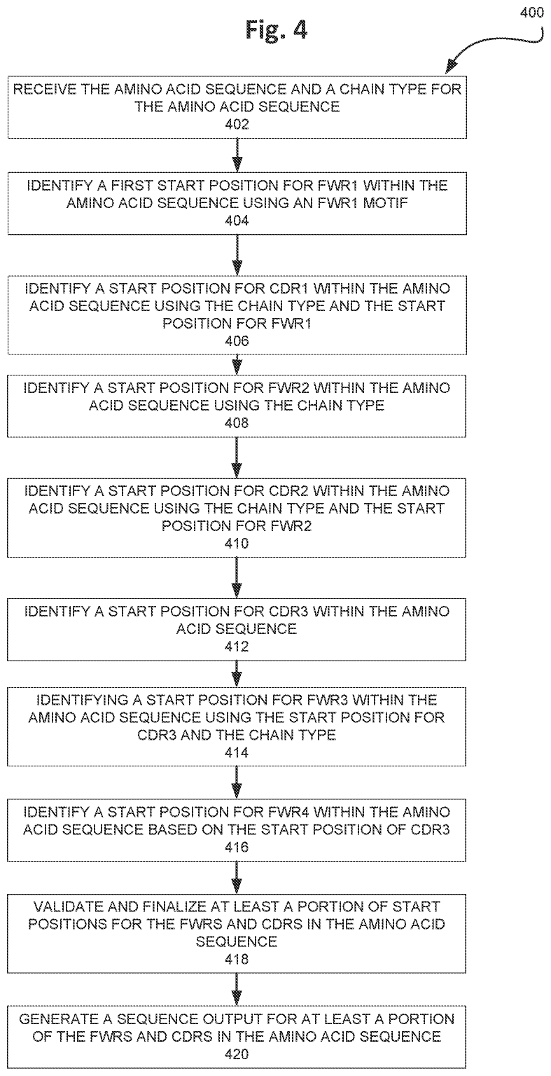

FIG. 4 is a flowchart of a process for locating FWRs and CDRs in an amino acid sequence in accordance with one or more embodiments. Process 400 is one example an embodiment for locating FWRs and CDRs using motifs. In one or more embodiments, process 400 may be implemented using, for example, without limitation, computing platform 142 in FIG. 1.

Step 402 includes receiving an amino acid sequence and a chain type for the amino acid is associated. The amino acid sequence is a reference sequence that may be generated using, for example, workflow 100 in FIG. 1. In one or more embodiments, the amino acid sequence is included in a sequencing dataset generated for an immunoglobulin chain (e.g., heavy chain, lambda light chain, kappa light chain.). In one or more embodiments, the amino acid sequence is included in a sequencing dataset generated for T cell receptor chain (e.g., alpha chain, beta chain). In one or more embodiments, the amino acid sequence may be in an amino acid format, a nucleotide format, or an amino acid and nucleotide format. In one or more embodiments, each amino acid in the sequence corresponds to one position of the amino acid. These positions may be, for example, without limitation, numbered sequentially in the 5′ to 3′ direction.

Step 404 includes identifying a start position for FWR1 within an amino acid sequence. In various embodiments, step 404 includes using an FWR1 motif. In one or more embodiments, the FWR1 motif is a scoring motif that is used to score different positions of the amino acid sequence to indicate the likelihood that the positions are the start position for the FWR1 motif. In one or more embodiments, the FWR1 motif takes the form of a position-weight-matrix.

Step 406 includes identifying a start position for CDR1 within the amino acid sequence using the chain type and the start position for FWR1. In various embodiments, step 406 includes using an CDR1 motif. In one or more embodiments, the CDR1 motif is a scoring motif that is used to score different positions of the amino acid sequence to indicate the likelihood that the positions are the start position for the CDR1 motif. In one or more embodiments, the CDR1 motif takes the form of a position-weight-matrix.

Step 408 includes identifying a start position for FWR2 within the amino acid sequence using the chain type. In various embodiments, step 408 includes using an FWR2 motif. In one or more embodiments, the FWR2 motif is a scoring motif that is used to score different positions of the amino acid sequence to indicate the likelihood that the positions are the start position for the FWR2 motif. In one or more embodiments, the FWR2 motif takes the form of a position-weight-matrix.

Step 410 includes identifying a start position for CDR2 within the amino acid sequence using the chain type and the start position for FWR2. In various embodiments, step 410 includes using an CDR2 motif. In one or more embodiments, the CDR2 motif is a scoring motif that is used to score different positions of the amino acid sequence to indicate the likelihood that the positions are the start position for the CDR2 motif. In one or more embodiments, the CDR2 motif takes the form of a position-weight-matrix. In one or more embodiments, step 410 is performed without using a CDR2 motif. For example, step 410 may be performed for light chains and beta chains without using a CDR2. In one or more embodiments, the CDR2 motif used for a heavy chain is different from the CDR2 motif used for an alpha chain.

Step 412 includes identifying a start position for CDR3 within the amino acid sequence. In various embodiments, step 412 includes using an CDR3 motif. In one or more embodiments, the CDR3 motif is a scoring motif that is used to score different positions of the amino acid sequence to indicate the likelihood that the positions are the start position for the CDR3 motif. In one or more embodiments, the CDR3 motif takes the form of a position-weight-matrix.

Step 414 includes identifying a start position for FWR3 within the amino acid sequence using the start position for CDR3 and the chain type. In various embodiments, step 414 includes using an FWR3 motif. In one or more embodiments, the FWR3 motif is a scoring motif that is used to score different positions of the amino acid sequence to indicate the likelihood that the positions are the start position for the FWR3 motif. In one or more embodiments, the FWR3 motif takes the form of a position-weight-matrix. In one or more embodiments, the FWR3 motif selected for step 414 is selected based on the chain type.

Step 416 includes identifying a start position for FWR4 within the amino acid sequence based on the start position of CDR3. In various embodiments, step 416 includes identifying the start position for FWR4 using one or more FWR4 motifs (e.g., PWMs).

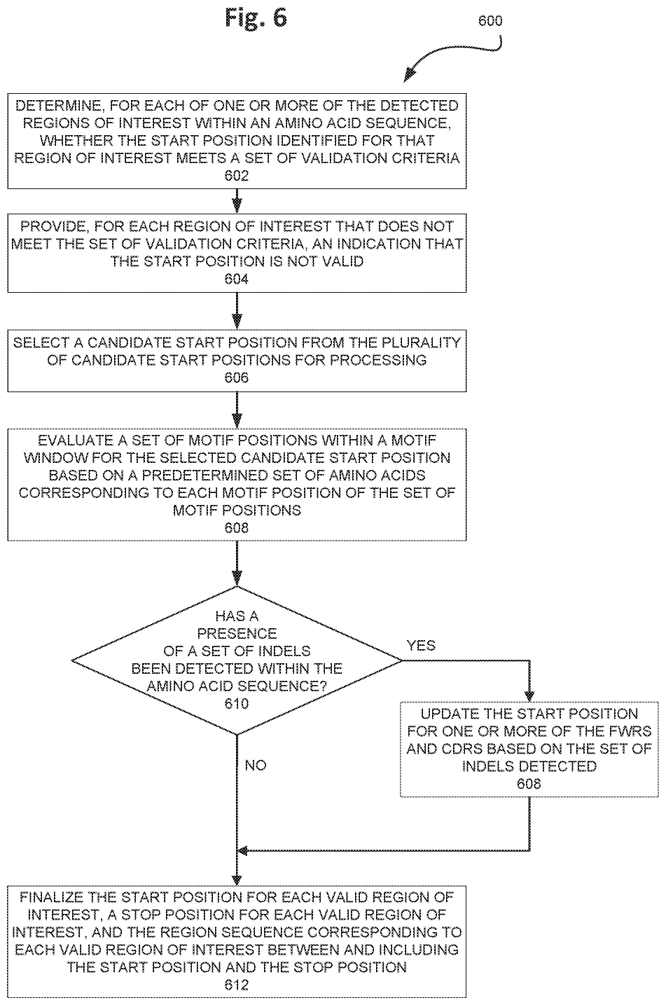

Step 418 includes validating and finalizing at least a portion of start positions for the FWRs and CDRs in the amino acid sequence. In one or more embodiments, step 418 includes validating the start position of FWR1, the start position of CDR1, the start position of FWR2, the start position of CDR2, the start position of FWR3, and the start position of CDR3, the start position of FWR4, or a combination thereof. One example of a manner in which step 418 may be implemented is described below in process 600 of FIG. 6.

Step 420 includes generating a sequence output for at least a portion of the FWRs and CDRs in the amino acid sequence. In various embodiments, this sequence output includes identifying sequences for a portion of the framework regions and complementarity-determining regions determined to have valid start positions. In one or more embodiments, the “region sequence” for a particular FWR or CDR may be returned in an amino acid format, a nucleotide format, or an amino acid and nucleotide format.

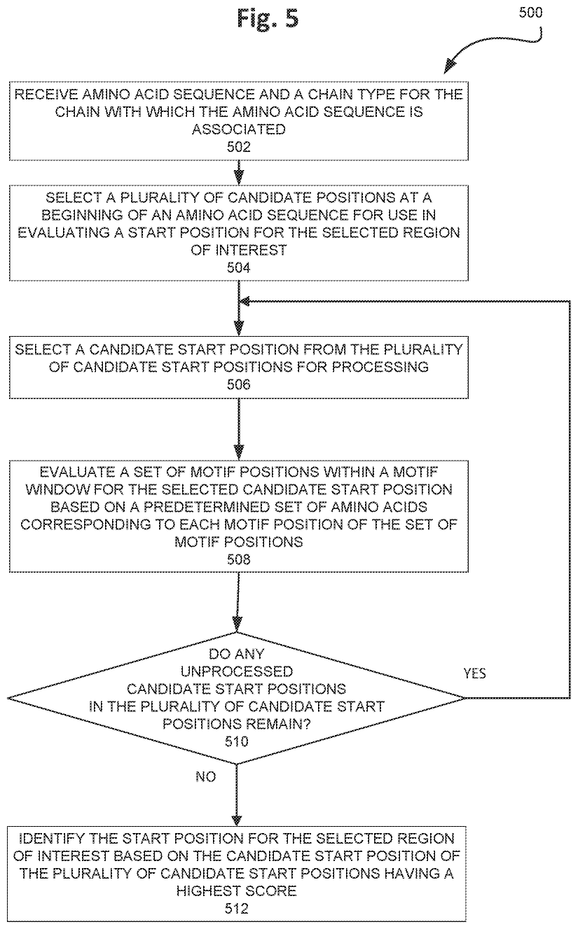

FIG. 5 is a flowchart of a process for identifying a start position for a selected region of interest in accordance with one or more embodiments. Process 500 is one example of a more detailed implementation of process 300 described in FIG. 3 above. In one or more embodiments, process 500 is one example of a manner in which one or more of operations 404-414 may be implemented. In one or more embodiments, process 500 may be implemented using, for example, without limitation, computing platform 142 in FIG. 1.

Step 502 includes receiving an amino acid sequence and a chain type for the chain with which the amino acid sequence is associated. The amino acid sequence is for the chain of an immune cell receptor. The chain type may be selected as one of: a heavy chain, a light chain (lambda light chain or kappa light chain), an alpha chain, or a beta chain.

Step 504 includes selecting a plurality of candidate positions at a beginning of an amino acid sequence for use in evaluating a start position for the selected region of interest. The selected region of interest may be a FWR or a CDR.

In one or more embodiments, when the selected region of interest is FWR1, step 504 includes selecting a first portion of the positions at the beginning of the amino acid sequence as the plurality of candidate start positions. In various embodiments, the first portion of the positions includes 50 positions at the beginning of the amino acid sequence, with each position having an amino acid at that position.

In one or more embodiments, when the selected region of interest is CDR1, step 504 includes selecting 9 positions that begin 19 positions after a previously identified start position for FWR1 as the plurality of candidate start positions. For example, the 19^{th} position of the amino acid sequence after the start position identified for FWR1 through the 27^{th} position of the amino acid sequence after the start position identified for FWR1 are used as the plurality of candidate start positions.

In one or more embodiments, when the selected region of interest is FWR2, step 504 includes selecting a first plurality of candidate start positions for when the amino acid sequence is associated with a heavy chain and a second plurality of candidate start positions for when the amino acid sequence is associated with a light chain, alpha chain or beta chain. In one or more embodiments, for a heavy chain, step 504 includes selecting 23 positions beginning at a 40^{th} position of the amino acid sequence as the plurality of candidate start positions. For example, positions 40-62 are used. In one or more embodiments, for a light chain, alpha chain, or beta chain, step 504 includes selecting 34 positions beginning at the 40^{th} position of the amino acid sequence as the plurality of candidate start positions. For example, positions 40-73 are used.

In one or more embodiments, when the selected region of interest is CDR2, step 504 includes selecting a first plurality of candidate start positions for when the amino acid sequence is associated with a heavy chain and a second plurality of candidate start positions for when the amino acid sequence is associated with an alpha chain. In one or more embodiments, for a heavy chain, step 504 includes selecting six positions after a previously identified start position for FWR2 as the plurality of candidate start positions. In one or more embodiments, for an alpha chain, step 504 includes selecting three positions after a previously identified start position for FWR2 as the plurality of candidate start positions.

In one or more embodiments, when the selected region of interest is CDR2 and when the amino acid sequence is associated with a light chain or beta chain, the start position for CDR2 may be determined directly based on the start position identified for FWR2. For example, without limitation, for a light chain, the start position for CDR2 may be identified as the 15^{th} position in the amino acid sequence after the start position for FWR2. For a beta chain, the start position for CDR2 may be identified as the 17^{th} position in the amino acid sequence after the start position for FWR2.

In one or more embodiments, when the selected region of interest is CDR3, all positions in the amino acid sequence are selected as the plurality of candidate start positions. In one or more embodiments, when the selected region of interest is FWR4, the start position for FWR4 is identified directly based on the start position identified for CDR3. In some embodiments, all positions in the amino acid sequence between the start position identified for CDR3 and a constant region of the amino acid sequence are selected as the plurality of candidate start positions.

In one or more embodiments, when the selected region of interest is FWR3, a different plurality of positions may be selected as the plurality of candidate start positions for each different possible chain type. In various embodiments, for a heavy chain, a 40^{th} position before a previously identified start position for a CDR3 through a 34^{th} position before the previously identified start position for CDR3 are selected as the plurality of candidate start positions. In various embodiments, for a light chain, a 35^{th} position before the previously identified start position for CDR3 through a 28^{th} position before the previously identified start position for CDR3 as the plurality of candidate start positions. In various embodiments, for an alpha chain, a 36^{th} position before the previously identified start position for CDR3 through a 33^{rd} position before the previously identified start position for CDR3 as the plurality of candidate start positions. In various embodiments, for a beta chain, a 38^{th} position before the previously identified start position for CDR3 through the 35^{th} position before the previously identified start position for CDR3 as the plurality of candidate start positions.

Step 506 includes selecting a candidate start position from the plurality of candidate start positions for processing. Step 506 begins an iterative process in which each candidate start position of the plurality of candidate start positions is associated with a different iteration. For each iteration, a motif window is analyzed for the corresponding candidate position. For example, for a given candidate start position, the motif window identifies relevant positions for evaluation beginning at the given candidate start position. In one or more embodiments, the candidate start position at which a motif windows begins is referred to as a corresponding candidate start position for that motif window.

Step 508 includes evaluating a set of motif positions within a motif window for the selected candidate start position based on a predetermined set of amino acids corresponding to each motif position of the set of motif positions. This evaluation may be performed for FWR1, CDR1, FWR2, CDR2, FWR3, or CD3. In some embodiments, this evaluation is performed for FWR4.

Evaluation Via FWR1 Motif:

In one or more embodiments, the motif window for FWR1 includes at least 23 motif positions. In one or more embodiments, step 508 includes evaluating six motif positions of the at least 23 motif positions within the motif window for the selected candidate start position based on a predetermined set of amino acids corresponding to each motif position of the six motif positions. In various embodiments, step 508 includes determining whether a corresponding amino acid at a 1^{st} motif position within the motif window matches one of the predetermined set of amino acids for the 1^{st} motif position, where the predetermined set of amino acids includes glutamine (Q), aspartic acid (D), glutamic acid (E), lysine (K), or glycine (G). In various embodiments, step 508 includes determining whether a corresponding amino acid at a 1^{st} motif position within the motif window is cysteine (C). In some embodiments, positions of the motif window are updated in response to a determination that the first position is cysteine. For example, the motif window may be shifted up by one such that for each original motif position after the 1^{st} motif position, a new motif position is increased in number by one from the original motif position.

In one or more embodiments, step 508 includes determining whether a corresponding amino acid at a 2^{nd} motif position within the motif window matches one of the predetermined set of amino acids for the 2^{nd} motif position, where the predetermined set of amino acids includes alanine (A), isoleucine (I), glutamine (Q), and valine (V). In one or more embodiments, step 508 includes determining whether a corresponding amino acid at a 4^{th} motif position within the motif window matches one of the predetermined set of amino acids for the 4^{th} motif position, where the predetermined set of amino acids includes leucine (L), methionine (M), and valine (V).

In various embodiments, step 508 includes determining whether a corresponding amino acid at a 6^{th} motif position within the motif window matches one of the predetermined set of amino acids for the 6^{th} motif position, where the predetermined set of amino acids includes glutamic acid (E) and glutamine (Q). In various embodiments, step 508 includes determining whether a corresponding amino acid at a 22^{nd} motif position within the motif window is cysteine (C). In various embodiments, step 508 includes determining whether a corresponding amino acid at a 23^{rd} motif position within the motif window is cysteine (C).

Evaluation Via CDR1 Motif:

In one or more embodiments, the motif window for CDR1 includes at least nine motif positions. In one or more embodiments, step 508 includes evaluating six motif positions of the at least nine motif positions within the motif window for the selected candidate start position based on a predetermined set of amino acids corresponding to each motif position of the six motif positions. In various embodiments, step 508 includes determining whether a corresponding amino acid at a 1^{st} motif position within the motif window is valine (V). In various embodiments, step 508 includes determining whether a corresponding amino acid at a 2^{nd} motif position within the motif window is threonine (T). In various embodiments, step 508 includes determining whether a corresponding amino acid at a 3^{rd} motif position within the motif window matches one of the predetermined set of amino acids for the 3^{rd} motif position, where the predetermined set of amino acids includes isoleucine (I), leucine (L), methionine (M), and valine (V).

In one or more embodiments, step 508 includes determining whether a corresponding amino acid at a 4^{th} motif position within the motif window matches one of the predetermined set of amino acids for the 4^{th} motif position, where the predetermined set of amino acids includes arginine (R), serine (S), and threonine (T). In one or more embodiments, step 508 includes determining whether a corresponding amino acid at a 5^{th} motif position within the motif window is cysteine (C). In one or more embodiments, step 508 includes determining whether a corresponding amino acid at an 8^{th} motif position within the motif window matches one of the predetermined set of amino acids for the 8^{th} motif position, where the predetermined set of amino acids includes isoleucine (I), serine (S), and aspartic acid (A).

Evaluation Via FWR2 Motif:

In one or more embodiments, the motif window for FWR2 includes at least 11 motif positions. In one or more embodiments, step 508 includes evaluating nine motif positions of the at least 11 motif positions within the motif window for the selected candidate start position based on a predetermined set of amino acids corresponding to each motif position of the nine motif positions. In various embodiments, step 508 includes determining whether a corresponding amino acid at a 1^{st} motif position within the motif window matches one of the predetermined set of amino acids for the 1^{st} motif position, where the predetermined set of amino acids includes phenylalanine (F), leucine (L), methionine (M), and valine (V).

In one or more embodiments, step 508 includes determining whether a corresponding amino acid at a 2^{nd} motif position within the motif window is tyrosine (Y). In one or more embodiments, step 508 includes determining whether a corresponding amino acid at a 3^{rd} motif position within the motif window is tryptophan (W). In one or more embodiments, step 508 includes determining whether a corresponding amino acid at a 4^{th} motif position within the motif window is tyrosine (Y). In one or more embodiments, step 508 includes determining whether a corresponding amino acid at a 5^{th} motif position within the motif window is arginine (R).

In various embodiments, step 508 includes determining whether a corresponding amino acid at a 6^{th} h motif position within the motif window is glutamine (Q). In one or more embodiments, step 508 includes determining whether a corresponding amino acid at a 9^{th} motif position within the motif window is glycine (G). In one or more embodiments, step 508 includes determining whether a corresponding amino acid at a 10^{th} motif position within the motif window matches one of the predetermined set of amino acids for the 10^{th} motif position, where the predetermined set of amino acids includes lysine (K) or glutamine (Q). In one or more embodiments, step 508 includes determining whether a corresponding amino acid at a 11^{th} motif position within the motif window matches one of the predetermined set of amino acids for the 11^{th} motif position, where the predetermined set of amino acids includes alanine (A), glycine (G), and lysine (K).

Evaluation Via CDR2 Motif:

In one or more embodiments, the motif window for CDR2 includes at least five motif positions. In one or more embodiments, step 508 includes evaluating the five motif positions within the motif window for the selected candidate start position based on a predetermined set of amino acids corresponding to each motif position of the five motif positions. In various embodiments, step 508 includes different determinations based on whether the chain type is a heavy chain or an alpha chain.

In one or more embodiments, for a heavy chain, step 508 includes determining whether a corresponding amino acid at a 1^{st} motif position within the motif window is leucine (L). In one or more embodiments, for a heavy chain, step 508 includes determining whether a corresponding amino acid at a 2^{nd} motif position within the motif window is glutamic acid (E). In one or more embodiments, for a heavy chain, step 508 includes determining whether a corresponding amino acid at a 3^{rd} motif position within the motif window is tryptophan (W). In one or more embodiments, for a heavy chain, step 508 includes determining whether a corresponding amino acid at a 4^{th} motif position within the motif window matches one of the predetermined set of amino acids for the 4^{th} motif position, where the predetermined set of amino acids includes isoleucine (I), leucine (L), methionine (M), and valine (V). In one or more embodiments, for a heavy chain, step 508 includes determining whether a corresponding amino acid at a 5^{th} motif position within the motif window matches one of the predetermined set of amino acids for the 4^{th} motif position, where the predetermined set of amino acids includes alanine (A), glycine (G), and serine (S).

In one or more embodiments, for an alpha chain, step 508 includes determining whether a corresponding amino acid at a 1^{st} motif position within the motif window matches one of the predetermined set of amino acids for the 1^{st} motif position, where the predetermined set of amino acids includes leucine (L) ad proline (P). In one or more embodiments, for an alpha chain, step 508 includes determining whether a corresponding amino acid at a 2^{nd} motif position within the motif window matches one of the predetermined set of amino acids for the 2^{nd} motif position, where the predetermined set of amino acids includes glutamic acid (E), isoleucine (I), glutamine (Q), threonine (T), and valine (V). In one or more embodiments, for an alpha chain, step 508 includes determining whether a corresponding amino acid at a 3^{rd} motif position within the motif window matches one of the predetermined set of amino acids for the 2^{nd} motif position, where the predetermined set of amino acids includes phenylalanine (F) and leucine (L). In one or more embodiments, for an alpha chain, step 508 includes determining whether a corresponding amino acid at a 4^{th} motif position within the motif window is leucine (L). In one or more embodiments, for an alpha chain, step 508 includes determining whether a corresponding amino acid at a 5^{th} motif position within the motif window matches one of the predetermined set of amino acids for the 2^{nd} motif position, where the predetermined set of amino acids includes isoleucine (I) and leucine (L).

Evaluation Via CDR3 Motif:

In one or more embodiments, the motif window for CDR3 includes at least 11 motif positions. In one or more embodiments, step 508 includes evaluating the 11 motif positions within the motif window for the selected candidate start position based on a predetermined set of amino acids corresponding to each motif position of the 11 motif positions. In various embodiments, step 508 includes determining whether a corresponding amino acid at a 1^{st} motif position within the motif window matches one of the predetermined set of amino acids for the 1^{st} motif position, where the predetermined set of amino acids includes alanine (A), leucine (L), and valine (V). In various embodiments, step 508 includes determining whether a corresponding amino acid at a 2^{nd} motif position within the motif window matches one of the predetermined set of amino acids for the 2^{nd} motif position, where the predetermined set of amino acids includes glutamic acid (E), glutamine (Q), and threonine (T).

In one or more embodiments, step 508 includes determining whether a corresponding amino acid at a 3^{rd} motif position within the motif window matches one of the predetermined set of amino acids for the 3^{rd} motif position, where the predetermined set of amino acids includes alanine (A), proline (P), and serine (S). In one or more embodiments, step 508 includes determining whether a corresponding amino acid at a 4^{th} motif position within the motif window matches one of the predetermined set of amino acids for the 3^{rd} motif position, where the predetermined set of amino acids includes glutamic acid (E), glycine (G), or serine (S). In one or more embodiments, step 508 includes determining whether a corresponding amino acid at a 5^{th} motif position within the motif window matches one of the predetermined set of amino acids for the 5^{th} motif position, where the predetermined set of amino acids includes aspartic acid (D) and glutamine (Q).

In one or more embodiments, step 508 includes determining whether a corresponding amino acid at a 6^{th} motif position within the motif window matches one of the predetermined set of amino acids for the 6^{th} motif position, where the predetermined set of amino acids includes alanine (A), serine (S), and threonine (T). In one or more embodiments, step 508 includes determining whether a corresponding amino acid at a 7^{th} motif position within the motif window matches one of the predetermined set of amino acids for the 7^{th} motif position, where the predetermined set of amino acids includes alanine (A), serine (S), and glycine (G). In one or more embodiments, step 508 includes determining whether a corresponding amino acid at an 8^{th} motif position within the motif window matches one of the predetermined set of amino acids for the 8^{th} motif position, where the predetermined set of amino acids includes leucine (L), threonine (T), and valine (V).

In one or more embodiments, step 508 includes determining whether a corresponding amino acid at a 9^{th} motif position within the motif window is tyrosine (Y). In one or more embodiments, step 508 includes determining whether a corresponding amino acid at a 10^{th} motif position within the motif window matches one of the predetermined set of amino acids for the 10^{th} motif position, where the predetermined set of amino acids includes phenylalanine (F), leucine (L), and tyrosine (Y). In one or more embodiments, step 508 includes determining whether a corresponding amino acid at a 11^{th} motif position within the motif window is cysteine (C). In one or more embodiments, step 508 includes determining whether a corresponding amino acid at a 5^{th} motif position within the motif window matches one of the predetermined set of amino acids for the 2^{nd} motif position, where the predetermined set of amino acids includes isoleucine (I) and leucine (L). In one or more embodiments, step 508 includes identifying the start position for the CDR2 as an 11^{th} motif position of the 11 motif positions for the candidate start position of the plurality of candidate start positions having the highest score.

Evaluation Via FWR3 Motif:

In one or more embodiments, the motif window for FWR3 for a heavy chain includes at least 10 motif positions. In one or more embodiments, the motif window for FWR3 for a light chain (e.g., lambda light chain or kappa light chain) includes at least 11 motif positions. In one or more embodiments, the motif window for FWR3 for a beta chain includes at least 12 motif positions. In one or more embodiments, the motif window for FWR3 for a heavy chain includes at least 12 motif positions.

In one or more embodiments, for a heavy chain, step 508 includes evaluating seven motif positions of the at least 10 motif positions within the motif window for the selected candidate start position based on a predetermined set of amino acids corresponding to each motif position of the seven motif positions. In various embodiments, for a heavy chain, step 508 includes determining whether a corresponding amino acid at a 1^{st} motif position within the motif window matches one of the predetermined set of amino acids for the 1^{st} motif position, where the predetermined set of amino acids includes asparagine (N) and tyrosine (Y).

In various embodiments, for a heavy chain, step 508 includes determining whether a corresponding amino acid at a 2^{nd} motif position within the motif window is tyrosine (Y). In one or more embodiments, for a heavy chain, step 508 includes determining whether a corresponding amino acid at a 3^{rd} motif position within the motif window matches one of the predetermined set of amino acids for the 3^{rd} motif position, where the predetermined set of amino acids includes alanine (A) and asparagine (N). In one or more embodiments, for a heavy chain, step 508 includes determining whether a corresponding amino acid at a 6^{th} position within the motif window matches one of the predetermined set of amino acids for the 6^{th} motif position, where the predetermined set of amino acids includes phenylalanine (F) and leucine (L).

In one or more embodiments, for a heavy chain, step 508 includes determining whether a corresponding amino acid at a 7^{th} position within the motif window matches one of the predetermined set of amino acids for the 7^{th} motif position, where the predetermined set of amino acids includes lysine (K), glutamine (Q), and arginine (R). In one or more embodiments, for a heavy chain, step 508 includes determining whether a corresponding amino acid at a 9^{th} position within the motif window matches one of the predetermined set of amino acids for the 9^{th} motif position, where the predetermined set of amino acids includes lysine (K) and arginine (R). In one or more embodiments, for a heavy chain, step 508 includes determining whether a corresponding amino acid at a 10^{th} position within the motif window matches one of the predetermined set of amino acids for the 10^{th} motif position, where the predetermined set of amino acids includes alanine (A), phenylalanine (F), valine (V), and leucine (L).

As described above, for a light chain, the motif window may include at least 8 motif positions. In one or more embodiments, for a light chain, step 508 includes evaluating five motif positions of the at least 8 motif positions within the motif window for the selected candidate start position based on a predetermined set of amino acids corresponding to each motif position of the five motif positions. In various embodiments, for a light chain, step 508 includes determining whether a corresponding amino acid at a 1^{st} motif position within the motif window is glycine (G).

In various embodiments, for a light chain, step 508 includes determining whether a corresponding amino acid at a 3^{rd} motif position within the motif window is proline (P). In various embodiments, for a light chain, step 508 includes determining whether a corresponding amino acid at a 5^{th} motif position within the motif window is arginine (R). In one or more embodiments, for a light chain, step 508 includes determining whether a corresponding amino acid at a 6^{th} motif position within the motif window is phenylalanine (F). In one or more embodiments, for a light chain, step 508 includes determining whether a corresponding amino acid at an 8^{th} motif position within the motif window is glycine (G).

As described above, for an alpha chain, the motif window may include at least 12 motif positions. In one or more embodiments, for an alpha chain, step 508 includes evaluating 11 motif positions of the at least 12 motif positions within the motif window for the selected candidate start position based on a predetermined set of amino acids corresponding to each motif position of the 11 motif positions. In one or more embodiments, for an alpha chain, step 508 includes determining whether a corresponding amino acid at a 1^{st} motif position within the motif window matches one of the predetermined set of amino acids for the 1^{st} motif position, where the predetermined set of amino acids includes glutamic acid (E), lysine (K), asparagine (N), and valine (V). In one or more embodiments, for an alpha chain, step 508 includes determining whether a corresponding amino acid at a 2^{nd} motif position within the motif window matches one of the predetermined set of amino acids for the 2^{nd} motif position, where the predetermined set of amino acids includes alanine (A), glutamic acid (E), lysine (K), and threonine (T).

In one or more embodiments, for an alpha chain, step 508 includes determining whether a corresponding amino acid at a 3^{rd} motif position within the motif window matches one of the predetermined set of amino acids for the 3^{rd} motif position, where the predetermined set of amino acids includes glutamic acid (E) and serine (S). In one or more embodiments, for an alpha chain, step 508 includes determining whether a corresponding amino acid at a 4^{th} motif position within the motif window matches one of the predetermined set of amino acids for the 4^{th} motif position, where the predetermined set of amino acids includes aspartic acid (D), asparagine (N), and serine (S). In various embodiments, for an alpha chain, step 508 includes determining whether a corresponding amino acid at a 5^{th} motif position within the motif window is asparagine (N). In various embodiments, for an alpha chain, step 508 includes determining whether a corresponding amino acid at a 6^{th} motif position within the motif window matches one of the predetermined set of amino acids for the 6^{th} motif position, where the predetermined set of amino acids includes glycine (G), methionine (M), and arginine (R).

In various embodiments, for an alpha chain, step 508 includes determining whether a corresponding amino acid at a 7^{th} motif position within the motif window matches one of the predetermined set of amino acids for the 7^{th} motif position, where the predetermined set of amino acids includes alanine (A), phenylalanine (F), isoleucine (I), and tyrosine (Y). In various embodiments, for an alpha chain, step 508 includes determining whether a corresponding amino acid at an 8^{th} motif position within the motif window matches one of the predetermined set of amino acids for the 8^{th} motif position, where the predetermined set of amino acids includes serine (S) and threonine (T).

In various embodiments, for an alpha chain, step 508 includes determining whether a corresponding amino acid at a 9^{th} motif position within the motif window matches one of the predetermined set of amino acids for the 9^{th} motif position, where the predetermined set of amino acids includes alanine (A) and valine (V). In one or more embodiments, for an alpha chain, step 508 includes determining whether a corresponding amino acid at a 10^{th} motif position within the motif window matches one of the predetermined set of amino acids for the 10^{th} motif position, where the predetermined set of amino acids includes glutamic acid (E) and threonine (T). In one or more embodiments, for an alpha chain, step 508 includes determining whether a corresponding amino acid at a 12^{th} motif position within the motif window matches one of the predetermined set of amino acids for the 12^{th} motif position, where the predetermined set of amino acids includes aspartic acid (D) and asparagine (N).

As described above, for a beta chain, the motif window may include 5 motif positions. In one or more embodiments, for a beta chain, step 508 includes evaluating the five motif positions within the motif window for the selected candidate start position based on a predetermined set of amino acids corresponding to each motif position of the five motif positions. In one or more embodiments, for a beta chain, step 508 includes determining whether a corresponding amino acid at a 1^{st} motif position within the motif window matches one of the predetermined set of amino acids for the 1^{st} motif position, wherein the predetermined set of amino acids includes aspartic acid (D), glutamic acid (E), and lysine (K).

In one or more embodiments, for a beta chain, step 508 includes determining whether a corresponding amino acid at a 2^{nd} motif position within the motif window matches one of the predetermined set of amino acids for the 2^{nd} motif position, wherein the predetermined set of amino acids includes glycine (G), glutamine (Q), and serine (S). In one or more embodiments, for a beta chain, step 508 includes determining whether a corresponding amino acid at a 3^{rd} motif position within the motif window matches one of the predetermined set of amino acids for the 3^{rd} motif position, wherein the predetermined set of amino acids includes aspartic acid (D), glutamic acid (E), glycine (G), and serine (S).

In various embodiments, for a beta chain, step 508 includes determining whether a corresponding amino acid at a 4^{th} motif position within the motif window matches one of the predetermined set of amino acids for the 4^{th} motif position, wherein the predetermined set of amino acids includes isoleucine (I), leucine (L), methionine (M), and valine (V). In various embodiments, for a beta chain, step 508 includes determining whether a corresponding amino acid at a 5^{th} motif position within the motif window matches one of the predetermined set of amino acids for the 5^{th} motif position, wherein the predetermined set of amino acids includes proline (P) and serine (S).

Step 508 includes updating a score for the corresponding candidate start position for each motif position of the set of motif positions that matches an amino acid in the set of amino acids selected for the position. Updating the score may include, for example, without limitation, adding a number of points to the score or subtracting a number of points from the score. In some embodiments, an update to the score adds zero points to the score.

In one or more embodiments, when the first motif position within the motif window is cysteine, the remaining motif positions of the motif window are modified. For example, without limitation, the numbers of the remaining motif positions may be increased by one. For example, the second motif position may become the third motif position, the third motif position may become the fourth position, etc. In one or more embodiments, the different motif positions in the set of motif positions may be weighted differently. For example, without limitation, a sixth motif position within the motif window matching one of a predetermined set of amino acids for the first motif position may be weighted higher than the second motif position matching one of a predetermined set of amino acids for the second motif position.

Step 510 includes determining whether any unprocessed candidate start positions in the plurality of candidate start positions remain. If no unprocessed candidate start positions remain, step 512 is performed, with step 512 including identifying the start position for the selected region of interest based on the candidate start position of the plurality of candidate start positions having a highest score. Otherwise, if any unprocessed candidate start positions remain, process 500 returns to step 506 as described above.

When the selected region of interest is FWR1, step 512 includes identifying the start position for the FWR1 as a 1^{st} motif position of the motif window at the candidate start position of the plurality of candidate start positions having the highest score. In other words, the amino acid at the candidate start position having the highest score is used as the start position for FWR1.

When the selected region of interest is CDR1, identification of the start position depends on the chain type for the amino acid sequence. For example, in one or more embodiments, for a light chain, step 512 includes identifying the start position for the CDR1 as a 5^{th} position after the candidate start position of the plurality of candidate start positions having the highest score. In various embodiments, for a heavy chain, alpha chain, or beta chain, step 512 includes identifying the start position for the CDR1 as an 8^{th} position after the candidate start position of the plurality of candidate start positions having the highest score.

When the selected region of interest is FWR2, identification of the start position depends on the chain type for the amino acid sequence. For example, in one or more embodiments, for a heavy chain, step 512 includes identifying the start position for the FWR2 as a 1^{st} motif position of the motif window at the candidate start position of the plurality of candidate start positions having the highest score. In one or more embodiments, for a light chain, step 512 includes identifying the start position for the FWR2 as the 2^{nd} motif position of the motif window at the candidate start position of the plurality of candidate start positions having the highest score. In various embodiments, for an alpha chain or a beta chain, step 512 includes identifying the start position for the FWR2 as one position before a 1^{st} motif position of the motif window at the candidate start position of the plurality of candidate start positions having the highest score.

When the selected region of interest is CDR2, identification of the start position may depend on the chain type for the amino acid sequence. For example, in one or more embodiments, for a heavy chain, step 512 includes identifying the start position for the CDR2 as a 7^{th} position after a 1^{st} motif position of the motif window at the candidate start position of the plurality of candidate start positions having the highest score. In various embodiments, for an alpha chain, step 512 includes identifying the start position for the CDR2 as the 6^{th} position after 1^{st} motif position of the motif window at the candidate start position of the plurality of candidate start positions having the highest score when the amino acid sequence is associated with an alpha chain.

When the selected region of interest is CDR3, step 512 may include identifying the start position for the CDR2 as an 11^{th} motif position of the 11 motif positions for the candidate start position of the plurality of candidate start positions having the highest score.

When the selected region of interest is FWR3, identification of the start position may depend on the chain type for the amino acid sequence. For example, in one or more embodiments, for a heavy chain, step 512 includes identifying the start position for the FWR3 as a 1^{st} motif position of the motif window at the candidate start position of the plurality of candidate start positions having the highest score. In one or more embodiments, for a light chain, step 512 includes identifying the start position for the FWR3 as a 1^{st} motif position of the motif window at the candidate start position of the plurality of candidate start positions having the highest score.

In one or more embodiments, for an alpha chain, step 512 includes identifying the start position for the FWR3 as one position before a 1^{st} motif position of the motif window at the candidate start position of the plurality of candidate start positions having the highest score. In one or more embodiments, for a beta chain, step 512 includes identifying the start position for the FWR3 as two positions before a 1^{st} motif position of the motif window at the candidate start position of the plurality of candidate start positions having the highest score.

FIG. 6 is a flowchart of a process for generating a sequence output identifying one or more FWRs, one or more CDRs, or both in an amino acid sequence in accordance with various embodiments. Process 600 may be one example of a manner in which step 418 in FIG. 4 may be implemented. In one or more embodiments, process 600 may be implemented using, for example, without limitation, computing platform 142 in FIG. 1.

Step 602 includes, for each of one or more of the detected regions of interest within an amino acid sequence, determining whether the start position identified for that region of interest meets a set of validation criteria. In one or more embodiments, step 602 may include performing this validation for FWR1, CDR1, FWR2, CDR2, FWR3, CDR3, or a combination thereof.

In various embodiments, the set of validation criteria includes one or more criteria to ensure that the start position identified for a FWR or CDR, as described above, is a reasonable or valid start position for that FWR or CDR. For example, the set of validation criteria may include a requirement that each region (e.g., FWR or CDR), that has a region adjacent to it in the 3′ direction of the amino acid sequence, does not have a start position that is higher (e.g., based on numbering, indexing, etc.) than the start position of this adjacent region. For example, without limitation, step 602 may include identifying whether an FWR (e.g., FWR1) has a start position that is greater than the start position of the adjacent CDR (e.g., CDR1) in the 3′ direction.

Step 604 includes, for each region of interest that does not meet the set of validation criteria, providing an indication that the start position is not valid. In one or more embodiments, this indication may be the return of a null region sequence or no region sequence for that region of interest.

Step 606 includes determining whether a presence of a set of indels is detected within the amino acid sequence. An indel is a mutation that is comprised of an insertion, a deletion, or both. If a set of indels is detected, step 608 is performed, with step 608 including updating the start position for one or more of the FWRs and CDRs based on the set of indels detected. For example, the start position for one or more of FWR1, CDR1, FWR2, CDR2, FWR3, CDR3, and FWR4 may be updated. The updating may include, for example, without limitation, shifting the start positions for one or more of the FWRs, one or more of the CDRs, or both by one or more positions. In some embodiments, the start position for one region in the amino acid sequence may be shifted by a different number of positions as compared to the start position for another region in the amino acid sequence.

Step 610 includes finalizing the start position for each valid region of interest, a stop position for each valid region of interest, and the region sequence corresponding to each valid region of interest between and including the start position and the stop position. With reference again to step 606, if no indels are detected, process 600 proceeds directly to step 610.

In describing the various embodiments, a method and/or process may be described as an exemplary sequence of steps. However, the method or process should not be limited to the particular sequence of steps described, and one skilled in the art can readily appreciate that the sequences may be varied and still remain within the spirit and scope of the various embodiments. For example, one or more steps may be performed simultaneously, in a reverse order, or in some other order that is varied from the order presented herein.

### Example PWMs

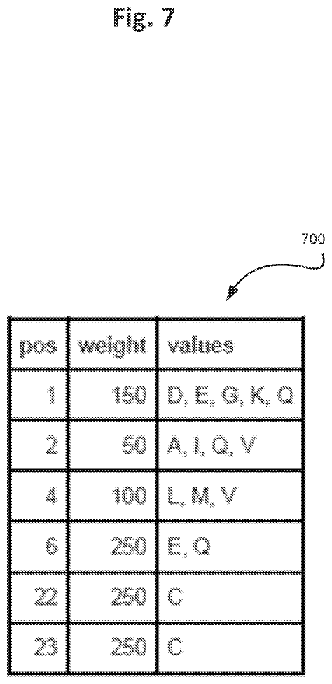

FIG. 7 is an illustration of a position weight matrix for FWR1 in accordance with various embodiments. PWM 700 is one example of a FWR1 motif that may be used to identify the sequence for FWR1. In one or more embodiments, PWM 700 is one example of a FWR1 motif that may be used to implement step 404 in FIG. 4 described above.

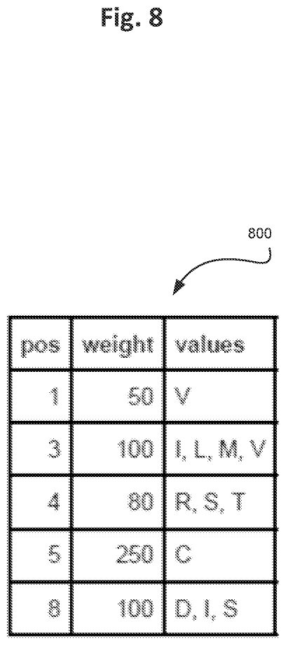

FIG. 8 is an illustration of a position weight matrix for CDR1 in accordance with various embodiments. PWM 800 is one example of a CDR1 motif that may be used to identify the sequence for CDR1. In one or more embodiments, PWM 800 is one example of a CDR1 motif that may be used to implement step 406 in FIG. 4 described above.

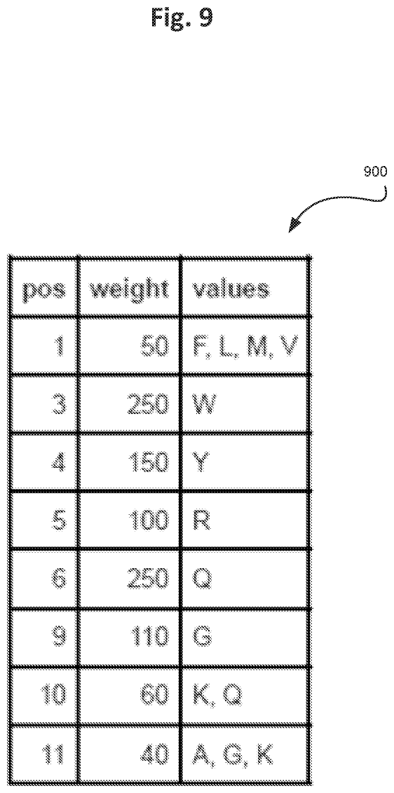

FIG. 9 is an illustration of a position weight matrix for FWR2 in accordance with various embodiments. PWM 900 is one example of a FWR2 motif that may be used to identify the sequence for FWR2. In one or more embodiments, PWM 900 is one example of a FWR2 motif that may be used to implement step 408 in FIG. 4 described above.

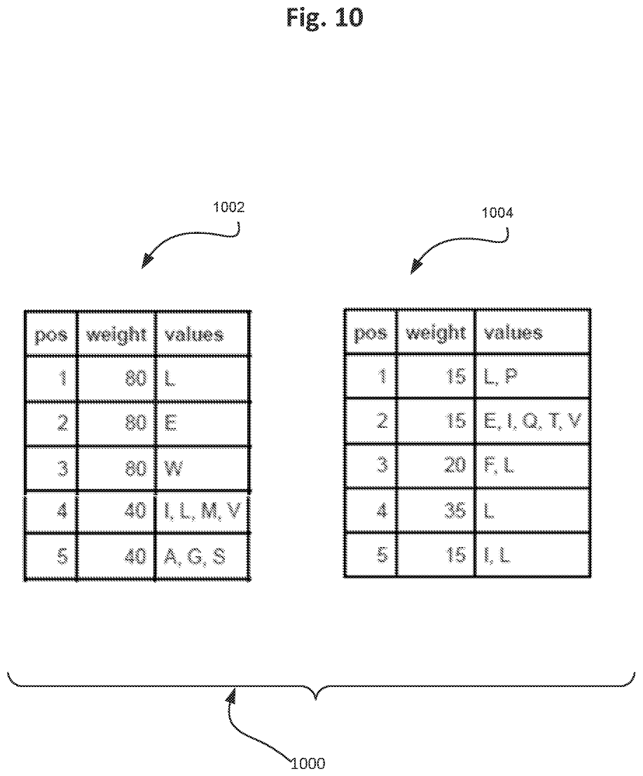

FIG. 10 is an illustration of a set of position weight matrices for CDR2 in accordance with various embodiments. In one or more embodiments, each PWM in set of PWMs 1000 is one example of a CDR2 motif that may be used to identify the sequence for CDR2. In one or more embodiments, each PWM in set of PWMs 1000 is one example of a CDR2 motif that may be used to implement step 410 in FIG. 4 described above.

In various embodiments, set of PWMs 1000 includes PWM 1002 and PWM 1004. PWM 1002 is one example of a CDR2 motif that may be used to identify the sequence for CDR2 when the given amino acid sequence is associated with a heavy chain. PWM 1004 is one example of a CDR2 motif that may be used to identify the sequence for CDR2 when the given amino acid sequence is associated with a light chain, alpha chain, or beta chain.

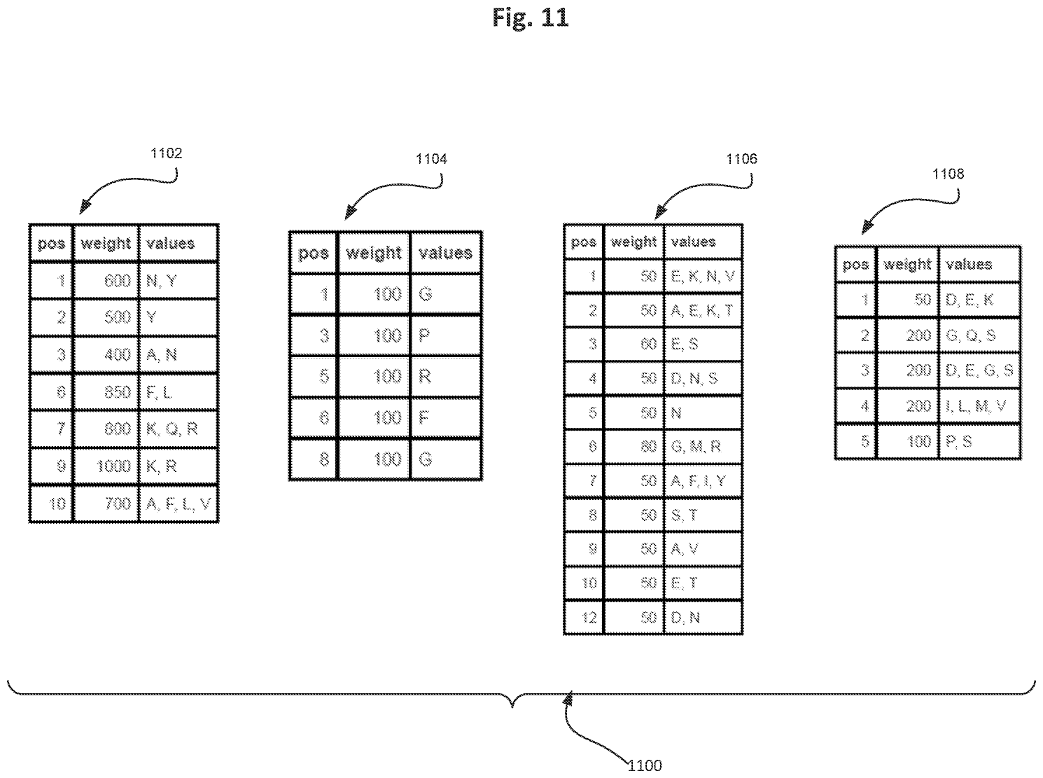

FIG. 11 is an illustration of a set of position weight matrices for FWR3 in accordance with various embodiments. In one or more embodiments, each PWM in set of PWMs 1100 is one example of a FWR3 motif that may be used to identify the sequence for FWR3. In one or more embodiments, each PWM in set of PWMs 1100 is one example of a FWR3 motif that may be used to implement step 414 in FIG. 4 described above.

In various embodiments, set of PWMs 1100 includes PWM 1102, PWM 1004, PWM 1006, and PWM 1006. PWM 1102 is one example of a FWR3 motif that may be used to identify the sequence for FWR3 when the given amino acid sequence is associated with a heavy chain. PWM 1104 is one example of a FWR3 motif that may be used to identify the sequence for FWR3 when the given amino acid sequence is associated with a light chain (e.g., lambda light chain, kappa light chain). PWM 1106 is one example of a FWR3 motif that may be used to identify the sequence for FWR3 when the given amino acid sequence is associated with an alpha chain. PWM 1108 is one example of a FWR3 motif that may be used to identify the sequence for FWR3 when the given amino acid sequence is associated with a beta chain.

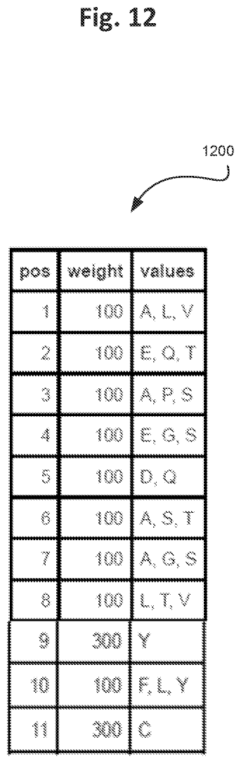

FIG. 12 is an illustration of a position weight matrix for CDR3 in accordance with various embodiments. PWM 1200 is one example of a CDR3 motif that may be used to identify the sequence for CDR3. In one or more embodiments, PWM 1200 is one example of a CDR3 motif that may be used to implement step 412 in FIG. 4 described above.

The PWMs shown in FIGS. 7-12 are exemplary only and are not meant to imply any architectural, functional, or computational limitations to the manner in which a PWM for a FWR or CDR may be implemented. For example, without limitation, in other embodiments, other types of weights (or points) may be applied to a particular motif position.

While the exemplary methods described above have been generally described with respect to single cell datasets, such methods or similar methods and visualization schemas may be used for spatial datasets, where a spatial dataset includes a dataset of at least one of immune cell receptors, antibodies, or fragments thereof from a tissue sample. For example, the spatial dataset may include a dataset of an antigen binding molecule (ABM), e.g., B cell receptor, a T cell receptor, an antibody, a single-chain variable fragment (ScFv), an antigen-binding fragment (Fab), or a combination thereof as obtained from a sample (e.g., a tissue sample).

### V. Computer System

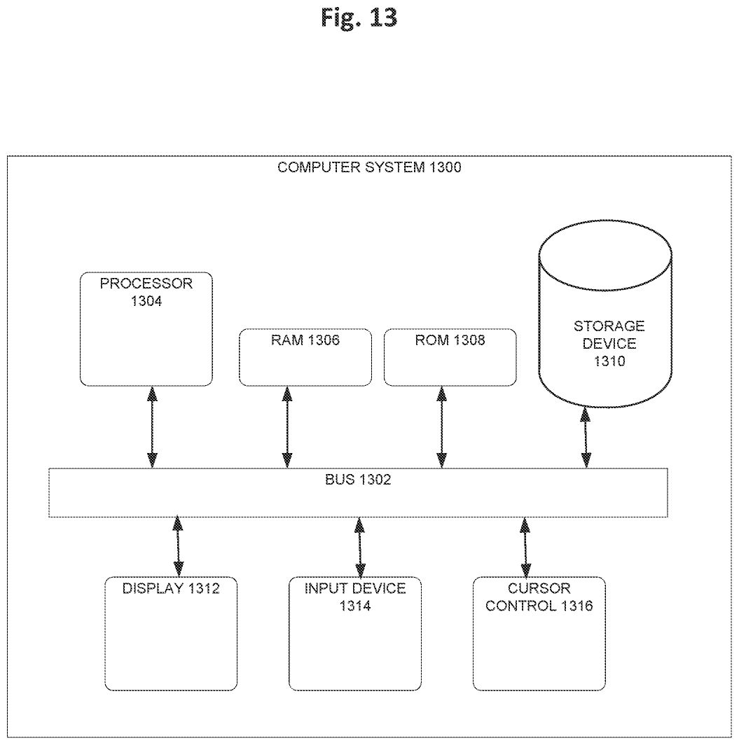

FIG. 13 is a block diagram that illustrates a computer system 1300 in accordance with various embodiments of the present disclosure. In one or more embodiments, computer system 1300 may be used to implement computing platform 142 in FIG. 1.

In various embodiments of the present disclosure, computer system 1300 can include a bus 1302 or other communication mechanism for communicating information, and a processor 1304 coupled with bus 1302 for processing information. In various embodiments, computer system 1300 can also include a memory, which can be a random access memory (RAM) 1306 or other dynamic storage device, coupled to bus 1302 for determining instructions to be executed by processor 1304. Memory also can be used for storing temporary variables or other intermediate information during execution of instructions to be executed by processor 1304. In various embodiments, computer system 1300 can further include a read only memory (ROM) 1308 or other static storage device coupled to bus 1302 for storing static information and instructions for processor 1304. A storage device 1310, such as a magnetic disk or optical disk, can be provided and coupled to bus 1302 for storing information and instructions.

In various embodiments, computer system 1300 can be coupled via bus 1302 to a display 1312, such as a cathode ray tube (CRT) or liquid crystal display (LCD), for displaying information to a computer user. An input device 1314, including alphanumeric and other keys, can be coupled to bus 1302 for communicating information and command selections to processor 1304. Another type of user input device is a cursor control 1316, such as a mouse, a trackball or cursor direction keys for communicating direction information and command selections to processor 1304 and for controlling cursor movement on display 1312. This input device 1314 typically has two degrees of freedom in two axes, a first axis (i.e., x) and a second axis (i.e., y), that allows the device to specify positions in a plane. However, it should be understood that input devices 1314 allowing for 3-dimensional (x, y and z) cursor movement are also contemplated herein.

Consistent with certain implementations of the present disclosure, results can be provided by computer system 1300 in response to processor 1304 executing one or more sequences of one or more instructions contained in memory 1306. Such instructions can be read into memory 1306 from another computer-readable medium or computer-readable storage medium, such as storage device 1310. Execution of the sequences of instructions contained in memory 1306 can cause processor 1304 to perform the processes described herein. Alternatively, hard-wired circuitry can be used in place of or in combination with software instructions to implement the present disclosure. Thus, implementations of the present disclosure are not limited to any specific combination of hardware circuitry and software.

The term “computer-readable medium” (e.g., data store, data storage, etc.) or “computer-readable storage medium” as used herein refers to any media that participates in providing instructions to processor 1304 for execution. Such a medium can take many forms, including but not limited to, non-volatile media, volatile media, and transmission media. Examples of non-volatile media can include, but are not limited to, optical, solid state, magnetic disks, such as storage device 1310. Examples of volatile media can include, but are not limited to, dynamic memory, such as memory 1306. Examples of transmission media can include, but are not limited to, coaxial cables, copper wire, and fiber optics, including the wires that comprise bus 1302.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 1304 of computer system 1300 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flow charts, diagrams and accompanying disclosure can be implemented using computer system 1300 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present disclosure may be implemented as firmware and/or a software program and applications written in conventional programming languages such as C, C++, Python, Rust, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1300 of Appendix D, whereby processor 1304 would execute the analyses and determinations provided by these engines, subject to instructions provided by any one of, or a combination of, memory components 1306/408/410 and user input provided via input device 1314.

### Digital Processing Device

In various embodiments, the systems and methods described herein can include a digital processing device or use of the same. In various embodiments, the digital processing device can include one or more hardware central processing units (CPUs) or general-purpose graphics processing units (GPGPUs) that carry out the device's functions. In various embodiments, the digital processing device further comprises an operating system configured to perform executable instructions. In various embodiments, the digital processing device can be optionally connected a computer network. In various embodiments, the digital processing device can be optionally connected to the Internet such that it accesses the World Wide Web. In various embodiments, the digital processing device can be optionally connected to a cloud computing infrastructure. In various embodiments, the digital processing device can be optionally connected to an intranet. In various embodiments, the digital processing device can be optionally connected to a data storage device.

In accordance with various embodiments, suitable digital processing devices can include, by way of non-limiting examples, server computers, desktop computers, laptop computers, notebook computers, sub-notebook computers, netbook computers, netpad computers, handheld computers, Internet appliances, mobile smartphones, tablet computers, and personal digital assistants. Those of ordinary skill in the art will recognize that many smartphones are suitable for use in the system described herein. Those of ordinary skill in the art will also recognize that select televisions, video players, and digital music players with optional computer network connectivity are suitable for use in the system described herein. Suitable tablet computers include those with booklet, slate, and convertible configurations, known to those of ordinary skill in the art.

In various embodiments, the digital processing device includes an operating system configured to perform executable instructions. The operating system can be, for example, software, including programs and data, which manages the device's hardware and provides services for execution of applications. Those of ordinary skill in the art will recognize that suitable server operating systems include, by way of non-limiting examples, FreeBSD, OpenBSD, Net-BSD, Linux, Apple® Mac OS X Server®, Oracle® Solaris®, Windows Server®, and Novell® NetWare®. Those of ordinary skill in the art will recognize that suitable personal computer operating systems include, by way of non-limiting examples, Microsoft® Windows®, Apple® Mac OS X®, UNIX®, and UNIX-like operating systems such as GNU/Linux®. In various embodiments, the operating system is provided by cloud computing. Those of ordinary skill in the art will also recognize that suitable mobile smart phone operating systems include, by way of non-limiting examples, Nokia® Symbian® OS, Apple® iOS®, Research In Motion® Black-Berry OS®, Google® Android®, Microsoft® Windows Phone® OS, Microsoft® Windows Mobile® OS, Linux®, and Palm® WebOS®.

In various embodiments, the device includes a storage and/or memory device. The storage and/or memory device is one or more physical apparatuses used to store data or programs on a temporary or permanent basis. In various embodiments, the device is volatile memory and requires power to maintain stored information. In various embodiments, the device is non-volatile memory and retains stored information when the digital processing device is not powered. In various embodiments, the non-volatile memory comprises flash memory. In some embodiments, the non-volatile memory comprises dynamic random-access memory (DRAM). In various embodiments, the non-volatile memory comprises ferroelectric random-access memory (FRAM). In various embodiments, the non-volatile memory comprises phase-change random access memory (PRAM). In various embodiments, the device is a storage device including, by way of non-limiting examples, CD-ROMs, DVDs, flash memory devices, magnetic disk drives, magnetic tapes drives, optical disk drives, and cloud computing-based storage. In various embodiments, the storage and/or memory device is a combination of devices such as those disclosed herein.

In various embodiments, the digital processing device includes a display to send visual information to a user. In various embodiments, the display is a cathode ray tube (CRT). In various embodiments, the display is a liquid crystal display (LCD). In various embodiments, the display is a thin film transistor liquid crystal display (TFT-LCD). In various embodiments, the display is an organic light emitting diode (OLED) display. In various embodiments, on OLED display is a passive-matrix OLED (PMOLED) or active-matrix OLED (AMOLED) display. In various embodiments, the display is a plasma display. In various embodiments, the display is a video projector. In various embodiments, the display is a combination of devices such as those disclosed herein.

In various embodiments, the digital processing device includes an input device to receive information from a user. In various embodiments, the input device is a keyboard. In various embodiments, the input device is a pointing device including, by way of non-limiting examples, a mouse, trackball, track pad, joystick, game controller, or stylus. In various embodiments, the input device is a touch screen or a multi-touch screen. In various embodiments, the input device is a microphone to capture voice or other sound input. In various embodiments, the input device is a video camera or other sensor to capture motion or visual input. In various embodiments, the input device is a Kinect, Leap Motion, or the like. In various embodiments, the input device is a combination of devices such as those disclosed herein.

### Non-Transitory Computer Readable Storage Medium

In various embodiments, and as stated above, the systems and methods disclosed herein can include, and the methods herein can be run on, one or more non-transitory computer readable storage media encoded with a program including instructions executable by the operating system of an optionally networked digital processing device. In various embodiments, a computer readable storage medium is a tangible component of a digital processing device. In various embodiments, a computer readable storage medium is optionally removable from a digital processing device. In various embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In various embodiments, the program and instructions are permanently, substantially permanently, semi-permanently, or non-transitorily encoded on the media.

### Computer Program

In various embodiments, the systems and methods disclosed herein can include at least one computer program or use at least one computer program. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. Those of ordinary skill in the art will recognize that a computer program may be written in various versions of various languages.

The functionality of the computer readable instructions may be combined or distributed as desired in various environments. In various embodiments, a computer program comprises one sequence of instructions. In various embodiments, a computer program comprises a plurality of sequences of instructions. In various embodiments, a computer program is provided from one location. In various embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In various embodiments, a computer program includes a web application. Those of ordinary skill in the art will recognize that a web application, in various embodiments, utilizes one or more software frameworks and one or more database systems. In various embodiments, a web application is created upon a software framework such as Microsoft® NET or Ruby on Rails (RoR). In various embodiments, a web application utilizes one or more database systems including, by way of non-limiting examples, relational, non-relational, object oriented, associative, and XML database systems. In various embodiments, suitable relational database systems include, by way of non-limiting examples, Microsoft® SQL Server, mySQL™, and Oracle®. Those of ordinary skill in the art will also recognize that a web application, in various embodiments, is written in one or more versions of one or more languages. A web application may be written in one or more markup languages, presentation definition languages, client-side scripting languages, server-side coding languages, data-base query languages, or combinations thereof. In various embodiments, a web application is written to some extent in a markup language such as Hypertext Markup Language (HTML), Extensible Hypertext Markup Language (XHTML), or eXtensible Markup Language (XML). In various embodiments, a web application is written to some extent in a presentation definition language such as Cascading Style Sheets (CSS). In various embodiments, a web application is written to some extent in a client-side scripting language such as Asynchronous Javascript and XML (AJAX), Flash® Actionscript, Javascript, or Silverlight®. In various embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tel, Smalltalk, WebDNA®, or Groovy. In various embodiments, a web application is written to some extent in a database query language such as Structured Query Language (SQL). In various embodiments, a web application integrates enterprise server products such as IBM® Lotus Domino®. In various embodiments, a web application includes a media player element. In various embodiments, a media player element utilizes one or more of many suitable multimedia technologies including, by way of non-limiting examples, Adobe® Flash®, HTML 5, Apple® QuickTime®, Microsoft® Silverlight®, Java™ and Unity®.

### Mobile Application

In various embodiments, a computer program includes a mobile application provided to a mobile digital processing device. In various embodiments, the mobile application is provided to a mobile digital processing device at the time it is manufactured. In various embodiments, the mobile application is provided to a mobile digital processing device via the computer network described herein.

A mobile application can be created by techniques known to those of ordinary skill in the art using hardware, languages, and development environments known to the art. Those of ordinary skill in the art will recognize that mobile applications can be written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelera-tor®, Celsius, Bedrock, Flash Lite, NET Compact Frame-work, Rhomobile, and WorkLight Mobile Platform. Other development environments are available without cost including, by way of non-limiting examples, Lazarus, Mobi-Flex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of ordinary skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo DSi Shop.

### Standalone Application

In various embodiments, a computer program includes a standalone application, which is a program that is run as an independent computer process, not an add-on to an existing process, e.g., not a plug-in. Those of ordinary skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Rust, Objective-C, COBOL, Delphi, Eiffel, Java™, Lisp, Python™, Visual Basic, and VB.NET, or combinations thereof. Compilation is often performed, at least in part, to create an executable program. In various embodiments, a computer program includes one or more executable complied applications.

### Web Browser Plug-In

In various embodiments, the computer program includes a web browser plug-in (e.g., extension, etc.). In computing, a plug-in is one or more software components that add specific functionality to a larger software application. Makers of software applications support plug-ins to enable third-party developers to create abilities, which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of ordinary skill in the art will be familiar with several web browser plug-ins including, Adobe® Flash® Player, Microsoft® Silver-Light®, and Apple® QuickTime®. In various embodiments, the toolbar comprises one or more web browser extensions, add-ins, or add-ons. In various embodiments, the toolbar comprises one or more explorer bars, tool bands, or desk bands.

Those of ordinary skill in the art will recognize that several plug-in frame works are available that enable development of plug-ins in various programming languages, including, by way of non-limiting examples, C++, Delphi, Java™, PHP, Python™, Rust, and VB NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Fire-Fox®, Google® Chrome, Apple® Safari®, Opera Software® Opera®, and KDE Konqueror. In various embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

### Software Modules

In various embodiments, the systems and methods disclosed herein include a software, server and/or database modules, or incorporate use of the same in methods according to various embodiments disclosed herein. Software modules can be created by techniques known to those of ordinary skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In various embodiments, software modules are in one computer program or application. In various embodiments, software modules are in more than one computer program or application. In various embodiments, software modules are hosted on one machine. In various embodiments, software modules are hosted on more than one machine. In various embodiments, software modules are hosted on cloud computing platforms. In various embodiments, software modules are hosted on one or more machines in one location. In various embodiments, software modules are hosted on one or more machines in more than one location.

### Databases

In various embodiments, the systems and methods disclosed herein include one or more databases, or incorporate use of the same in methods according to various embodiments disclosed herein. Those of ordinary skill in the art will recognize that many databases are suitable for storage and retrieval of user, query, token, and result information. In various embodiments, suitable databases include, by way of non-limiting examples, relational databases, non-relational databases, object-oriented databases, object databases, entity-relation-ship model databases, associative databases, and XML databases. Further non-limiting examples include SQL, Postgr-eSQL, MySQL, Oracle, DB2, and Sybase. In various embodiments, a database is internet-based. In further Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In various embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

In various embodiments, a database is web-based. In various embodiments, a database is cloud computing-based. In other embodiments, a database is based on one or more local computer storage devices.

### Data Security

In various embodiments, the systems and methods disclosed herein include one or features to prevent unauthorized access. The security measures can, for example, secure a user's data. In various embodiments, data is encrypted. In various embodiments, access to the system requires multi-factor authentication and access control layer. In various embodiments, access to the system requires two-step authentication (e.g., web-based interface). In various embodiments, two-step authentication requires a user to input an access code sent to a user's e-mail or cell phone in addition to a username and password. In some instances, a user is locked out of an account after failing to input a proper username and password. The systems and methods disclosed herein can, in various embodiments, also include a mechanism for protecting the anonymity of users' genomes and of their searches across any genomes.

### VI. Recitation of Embodiments

Embodiment 1. A method for identifying framework regions and complementarity-determining regions in an amino acid sequence, the method comprising: receiving the amino acid sequence; identifying a plurality of candidate start positions within the amino acid sequence for a start position for a selected region of interest; generating a score for each candidate start position of the plurality of candidate start positions via analysis of a motif window that begins at each candidate start position; and identifying the start position for the selected region of interest based on a candidate start position of the plurality of candidate start positions having a highest score.

Embodiment 2. The method of embodiment 1, wherein the selected region of interest is either a framework region or a complementarity-determining region.

Embodiment 3. The method of embodiment 1 or 2, wherein generating the score comprises: evaluating a set of motif positions within the motif window for a corresponding candidate start position of the plurality of candidate start positions based on a predetermined set of amino acids corresponding to each motif position of the set of motif positions; and updating the score for the corresponding candidate start position for each motif position in the set of motif positions that matches a corresponding amino acid in the predetermined set of amino acids that corresponds to each motif position.

Embodiment 4. The method of embodiment 3, wherein each motif position of the set of motif positions is weighted differently from at least one other motif position of the set of motif positions.

Embodiment 5. The method of any one of embodiments 1-4, wherein the selected region of interest is framework region 1 (FWR1).

Embodiment 6. The method of embodiment 5, wherein the identifying the plurality of candidate start positions comprises: selecting 50 positions at a beginning of the amino acid sequence as the plurality of candidate start positions.

Embodiment 7. The method of embodiment 5, wherein the motif window includes at least 23 motif positions and wherein generating the score comprises: evaluating six motif positions of the at least 23 motif positions within the motif window for a corresponding candidate start position of the plurality of candidate start positions based on a predetermined set of amino acids corresponding to each motif position of the six motif positions; and updating the score for the corresponding candidate start position for each motif position in the six motif positions that matches a corresponding amino acid in the predetermined set of amino acids that corresponds to each motif position.

Embodiment 8. The method of embodiment 7, wherein identifying the start position comprises: identifying the start position for the FWR1 as a 1^{st} motif position of the motif window at the candidate start position of the plurality of candidate start positions having the highest score.

Embodiment 9. The method of embodiment 7, wherein the evaluating comprises: determining whether a corresponding amino acid at a 1^{st} motif position within the motif window matches one of the predetermined set of amino acids for the 1^{st} motif position, wherein the predetermined set of amino acids includes glutamine (Q), aspartic acid (D), glutamic acid (E), lysine (K), or glycine (G).

Embodiment 10. The method of embodiment 7, wherein the evaluating comprises: determining whether a corresponding amino acid at a 1^{st} motif position within the motif window is cysteine (C).

Embodiment 11. The method of embodiment 10, further comprising: updating positions of the motif window in response to a determination that the 1^{st} motif position within the motif window is cysteine.

Embodiment 12. The method of any one of embodiments 7-11, wherein the evaluating comprises: determining whether a corresponding amino acid at a 2^{nd} motif position within the motif window matches one of the predetermined set of amino acids for the 2^{nd} motif position, wherein the predetermined set of amino acids includes alanine (A), isoleucine (I), glutamine (Q), and valine (V).

Embodiment 13. The method of any one of embodiments 7-11, wherein the evaluating comprises: determining whether a corresponding amino acid at a 4^{th} motif position within the motif window matches one of the predetermined set of amino acids for the 4^{th} motif position, wherein the predetermined set of amino acids includes leucine (L), methionine (M), and valine (V).

Embodiment 14. The method of any one of embodiments 7-13, wherein the evaluating comprises: determining whether a corresponding amino acid at a 6^{th} motif position within the motif window matches one of the predetermined set of amino acids for the 6^{th} motif position, wherein the predetermined set of amino acids includes glutamic acid (E) and glutamine (Q).

Embodiment 15. The method of any one of embodiments 7-14, wherein the evaluating comprises: determining whether a corresponding amino acid at a 22^{nd} motif position within the motif window is cysteine (C).

Embodiment 16. The method of any one of embodiments 7-15, wherein the evaluating comprises: determining whether a corresponding amino acid at a 23^{rd} motif position within the motif window is cysteine (C).

Embodiment 17. The method of any one of embodiments 7-16, wherein the selected region of interest is complementarity-determining region 1 (CDR1).

Embodiment 18. The method of embodiment 17, wherein identifying the plurality of candidate start positions comprises: selecting 9 positions that begin 19 positions after a previously identified start position for a framework region 1 (FWR1) as the plurality of candidate start positions.

Embodiment 19. The method of embodiment 17 or embodiment 18, wherein the motif window includes at least nine motif positions and wherein the generating the score comprises: evaluating six motif positions of the at least nine motif positions within the motif window for a corresponding candidate start position of the plurality of candidate start positions based on a predetermined set of amino acids corresponding to each motif position of the six motif positions; and updating the score for the corresponding candidate start position for each motif position in the six motif positions that matches a corresponding amino acid in the predetermined set of amino acids that corresponds to each motif position.

Embodiment 20. The method of embodiment 19, wherein identifying the start position comprises: identifying the start position for the CDR1 as a 5^{th} position after the candidate start position of the plurality of candidate start positions having the highest score when the amino acid sequence is associated with one of a lambda light chain or a kappa light chain; and identifying the start position for the CDR1 as an 8^{th} position after the candidate start position of the plurality of candidate start positions having the highest score when the amino acid sequence is associated with one of an alpha chain, a beta chain, or a heavy chain.

Embodiment 21. The method of any one of embodiments 19-20, wherein the evaluating comprises: determining whether a corresponding amino acid at a 1^{st} motif position within the motif window is valine (V).

Embodiment 22. The method of any one of embodiments 19-21, wherein the evaluating comprises: determining whether a corresponding amino acid at a 2^{nd} motif position within the motif window is threonine (T).

Embodiment 23. The method of any one of embodiments 19-22, wherein the evaluating comprises: determining whether a corresponding amino acid at a 3^{rd} motif position within the motif window matches one of the predetermined set of amino acids for the 3^{rd} motif position, wherein the predetermined set of amino acids includes isoleucine (I), leucine (L), methionine (M), and valine (V).

Embodiment 24. The method of any one of embodiments 19-23, wherein the evaluating comprises: determining whether a corresponding amino acid at a 4^{th} motif position within the motif window matches one of the predetermined set of amino acids for the 4^{th} motif position, wherein the predetermined set of amino acids includes arginine (R), serine (S), and threonine (T).

Embodiment 25. The method of any one of embodiments 19-24, wherein the evaluating comprises: determining whether a corresponding amino acid at a 5^{th} motif position within the motif window is cysteine (C).

Embodiment 26. The method of any one of embodiments 19-25, wherein the evaluating comprises: determining whether a corresponding amino acid at an 8^{th} motif position within the motif window matches one of the predetermined set of amino acids for the 8^{th} motif position, wherein the predetermined set of amino acids includes isoleucine (I), serine (S), and aspartic acid (A).

Embodiment 27. The method of any one of embodiments 1-26, wherein the selected region of interest is framework region 2 (FWR2).

Embodiment 28. The method of embodiment 27, wherein identifying the plurality of candidate start positions comprises: selecting 23 positions beginning with a 40^{th} position of the amino acid sequence as the plurality of candidate start positions when the amino acid sequence is associated with a heavy chain; and selecting 34 positions beginning with a 40^{th} position of the amino acid sequence as the plurality of candidate start positions when the amino acid sequence is associated with one of a lambda light chain, a kappa light chain, an alpha chain, or a beta chain.

Embodiment 29. The method of embodiment 27 or embodiment 28, wherein the motif window includes at least 11 motif positions and wherein the generating the score comprises: evaluating nine motif positions of the at least 11 motif positions within the motif window for a corresponding candidate start position of the plurality of candidate start positions based on a predetermined set of amino acids corresponding to each motif position of the nine motif positions; and updating the score for the corresponding candidate start position for each motif position in the nine motif positions that matches a corresponding amino acid in the predetermined set of amino acids that corresponds to each motif position.

Embodiment 30. The method of embodiment 29, wherein identifying the start position comprises: identifying the start position for the FWR2 as a 1^{st} motif position of the motif window at the candidate start position of the plurality of candidate start positions having the highest score when the amino acid sequence is associated with a heavy chain; identifying the start position for the FWR2 as a 2^{nd} motif position of the motif window at the candidate start position of the plurality of candidate start positions having the highest score when the amino acid sequence is associated with a light chain; and identifying the start position for the FWR2 as one position before a 1^{st} motif position of the motif window at the candidate start position of the plurality of candidate start positions having the highest score when the amino acid sequence is associated with an alpha chain or a beta chain.

Embodiment 31. The method of embodiment 29 or claim 30, wherein the evaluating comprises: determining whether a corresponding amino acid at a 1^{st} motif position within the motif window matches one of the predetermined set of amino acids for the 1^{st} motif position, wherein the predetermined set of amino acids includes phenylalanine (F), leucine (L), methionine (M), and valine (V).

Embodiment 32. The method of any one of embodiments 29-31, wherein the evaluating comprises: determining whether a corresponding amino acid at a 2^{nd} motif position within the motif window is tyrosine (Y).

Embodiment 33. The method of any one of embodiments 29-32, wherein the evaluating comprises: determining whether a corresponding amino acid at a 3^{rd} motif position within the motif window is tryptophan (W).

Embodiment 34. The method of any one of embodiments 29-33, wherein the evaluating comprises: determining whether a corresponding amino acid at a 4^{th} motif position within the motif window is tyrosine (Y).

Embodiment 35. The method of any one of embodiments 29-34, wherein the evaluating comprises: determining whether a corresponding amino acid at a 5^{th} motif position within the motif window is arginine (R).

Embodiment 36. The method of any one of embodiments 29-35, wherein the evaluating comprises: determining whether a corresponding amino acid at a 6^{th} motif position within the motif window is glutamine (Q).

Embodiment 37. The method of any one of embodiments 29-36, wherein the evaluating comprises: determining whether a corresponding amino acid at a 9^{th} motif position within the motif window is glycine (G).

Embodiment 38. The method of any one of embodiments 29-37, wherein the evaluating comprises: determining whether a corresponding amino acid at a 10^{th} motif position within the motif window matches one of the predetermined set of amino acids for the 10^{th} motif position, wherein the predetermined set of amino acids includes lysine (K) or glutamine (Q).

Embodiment 39. The method of any one of embodiments 29-38, wherein the evaluating comprises: determining whether a corresponding amino acid at a 11^{th} motif position within the motif window matches one of the predetermined set of amino acids for the 11^{th} motif position, wherein the predetermined set of amino acids includes alanine (A), glycine (G), and lysine (K).

Embodiment 40. The method of any one of embodiments 27-39 further comprising: identifying another start position for complementarity-determining region 2 (CDR2) as a 15^{th} position after the start position for FWR2 when the amino acid sequence is associated with a light chain.

Embodiment 41. The method of any one of embodiments 29-39 further comprising: identifying another start position for complementarity-determining region 2 (CDR2) as a 17^{th} position after the start position for FWR2 when the amino acid sequence is associated with a beta chain.

Embodiment 42. The method of any one of embodiments 1-41, wherein the selected region of interest is complementarity-determining region 2 (CDR2).

Embodiment 43. The method of embodiment 42, wherein identifying the plurality of candidate start positions comprises: selecting six positions after a previously identified start position for framework region 2 (FWR2) as the plurality of candidate start positions when the amino acid sequence is associated with a heavy chain; and selecting three positions after the previously identified start position for the FWR2 as the plurality of candidate start positions when the amino acid sequence is associated with one of an alpha chain.

Embodiment 44. The method of embodiment 42 or embodiment 43, wherein the motif window includes at least five motif positions and wherein the generating the score comprises: evaluating the five motif positions within the motif window for a corresponding candidate start position of the plurality of candidate start positions based on a predetermined set of amino acids corresponding to each motif position of the five motif positions; and updating the score for the corresponding candidate start position for each motif position in the five motif positions that matches a corresponding amino acid in the predetermined set of amino acids that corresponds to each motif position.

Embodiment 45. The method of embodiment 44, wherein identifying the start position comprises: identifying the start position for the CDR2 as a 7^{th} position after a 1^{st} motif position of the motif window at the candidate start position of the plurality of candidate start positions having the highest score when the amino acid sequence is associated with a heavy chain; and identifying the start position for the CDR2 as a 6^{th} position after 1^{st} motif position of the motif window at the candidate start position of the plurality of candidate start positions having the highest score when the amino acid sequence is associated with an alpha chain.

Embodiment 46. The method of embodiment 44 or embodiment 45, wherein the amino acid sequence is associated with a heavy chain and wherein the evaluating comprises: determining whether a corresponding amino acid at a 1^{st} motif position within the motif window is leucine (L).

Embodiment 47. The method of any one of embodiments 44-46, wherein the amino acid sequence is associated with a heavy chain and wherein the evaluating comprises: determining whether a corresponding amino acid at a 2^{nd} motif position within the motif window is glutamic acid (E).

Embodiment 48. The method of any one of embodiments 44-47, wherein the amino acid sequence is associated with a heavy chain and wherein the evaluating comprises: determining whether a corresponding amino acid at a 3^{rd} motif position within the motif window is tryptophan (W).

Embodiment 49. The method of any one of embodiments 44-48, wherein the amino acid sequence is associated with a heavy chain and wherein the evaluating comprises: determining whether a corresponding amino acid at a 4^{th} motif position within the motif window matches one of the predetermined set of amino acids for the 4^{th} motif position, wherein the predetermined set of amino acids includes isoleucine (I), leucine (L), methionine (M), and valine (V).

Embodiment 50. The method of any one of embodiments 44-49, wherein the amino acid sequence is associated with a heavy chain and wherein the evaluating comprises: determining whether a corresponding amino acid at a 5^{th} motif position within the motif window matches one of the predetermined set of amino acids for the 5^{th} motif position, wherein the predetermined set of amino acids includes alanine (A), glycine (G), and serine (S).

Embodiment 51. The method of any one of embodiments 44-50, wherein the amino acid sequence is associated with an alpha chain and wherein the evaluating comprises: determining whether a corresponding amino acid at a 1^{st} motif position within the motif window matches one of the predetermined set of amino acids for the 1^{st} motif position, wherein the predetermined set of amino acids includes leucine (L) ad proline (P).

Embodiment 52. The method of any one of embodiments 44-51, wherein the amino acid sequence is associated with an alpha chain and wherein the evaluating comprises: determining whether a corresponding amino acid at a 2^{nd} motif position within the motif window matches one of the predetermined set of amino acids for the 2^{nd} motif position, wherein the predetermined set of amino acids includes glutamic acid (E), isoleucine (I), glutamine (Q), threonine (T), and valine (V).

Embodiment 53. The method of any one of embodiments 44-52, wherein the amino acid sequence is associated with an alpha chain and wherein the evaluating comprises: determining whether a corresponding amino acid at a 3^{rd} motif position within the motif window matches one of the predetermined set of amino acids for the 3^{rd} motif position, wherein the predetermined set of amino acids includes phenylalanine (F) and leucine (L).

Embodiment 54. The method of any one of embodiments 44-53, wherein the amino acid sequence is associated with an alpha chain and wherein the evaluating comprises: determining whether a corresponding amino acid at a 4^{th} motif position within the motif window is leucine (L).

Embodiment 55. The method of any one of embodiments 44-54, wherein the amino acid sequence is associated with an alpha chain and wherein the evaluating comprises: determining whether a corresponding amino acid at a 5^{th} motif position within the motif window matches one of the predetermined set of amino acids for the 5^{th} motif position, wherein the predetermined set of amino acids includes isoleucine (I) and leucine (L).

Embodiment 56. The method of any one of embodiments 1-55, wherein the selected region of interest is complementarity-determining region 3 (CDR3).

Embodiment 57. The method of embodiment 56, wherein identifying the plurality of candidate start positions comprises: selecting all positions in the amino acid sequence as the plurality of candidate start positions.

Embodiment 58. The method of embodiment 56 or embodiment 57, wherein identifying the plurality of candidate start positions comprises: selecting all positions in the amino acid sequence after a previously identified start position for one of FWR1, CDR1, FWR2, or CDR2.

Embodiment 59. The method of any one of embodiments 56-58, wherein the motif window includes at least 11 motif positions and wherein the generating the score comprises: evaluating the 11 motif positions within the motif window for a corresponding candidate start position of the plurality of candidate start positions based on a predetermined set of amino acids corresponding to each motif position of the 11 motif positions; and updating the score for the corresponding candidate start position for each motif position in the 11 motif positions that matches a corresponding amino acid in the predetermined set of amino acids that corresponds to each motif position.

Embodiment 60. The method of embodiment 59, wherein the evaluating comprises: determining whether a corresponding amino acid at a 1^{st} motif position within the motif window matches one of the predetermined set of amino acids for the 1^{st} motif position, wherein the predetermined set of amino acids includes alanine (A), leucine (L), and valine (V).

Embodiment 61. The method of embodiment 59 or embodiment 60, wherein the evaluating comprises: determining whether a corresponding amino acid at a 2^{nd} motif position within the motif window matches one of the predetermined set of amino acids for the 2^{nd} motif position, wherein the predetermined set of amino acids includes glutamic acid (E), glutamine (Q), and threonine (T).

Embodiment 62. The method of any one of embodiments 59-61, wherein the evaluating comprises: determining whether a corresponding amino acid at a 3^{rd} motif position within the motif window matches one of the predetermined set of amino acids for the 3^{rd} motif position, wherein the predetermined set of amino acids includes alanine (A), proline (P), and serine (S).

Embodiment 63. The method of any one of embodiments 59-62, wherein the evaluating comprises: determining whether a corresponding amino acid at a 4^{th} motif position within the motif window matches one of the predetermined set of amino acids for the 4^{th} motif position, wherein the predetermined set of amino acids includes glutamic acid (E), glycine (G), or serine (S).

Embodiment 64. The method of any one of embodiments 59-63, wherein the evaluating comprises: determining whether a corresponding amino acid at a 5^{th} motif position within the motif window matches one of the predetermined set of amino acids for the 5^{th} motif position, wherein the predetermined set of amino acids includes aspartic acid (D) and glutamine (Q).

Embodiment 65. The method of any one of embodiments 59-64, wherein the evaluating comprises: determining whether a corresponding amino acid at a 6^{th} motif position within the motif window matches one of the predetermined set of amino acids for the 6^{th} motif position, wherein the predetermined set of amino acids includes alanine (A), serine (S), and threonine (T).

Embodiment 66. The method of any one of embodiments 59-65, wherein the evaluating comprises: determining whether a corresponding amino acid at a 7^{th} motif position within the motif window matches one of the predetermined set of amino acids for the 7^{th} motif position, wherein the predetermined set of amino acids includes alanine (A), serine (S), and glycine (G).

Embodiment 67. The method of any one of embodiments 59-66, wherein the evaluating comprises: determining whether a corresponding amino acid at an 8^{th} motif position within the motif window matches one of the predetermined set of amino acids for the 8^{th} motif position, wherein the predetermined set of amino acids includes leucine (L), threonine (T), and valine (V).

Embodiment 68. The method of any one of embodiments 59-67, wherein the evaluating comprises: determining whether a corresponding amino acid at a 9^{th} motif position within the motif window is tyrosine (Y).

Embodiment 69. The method of any one of embodiments 59-68, wherein the evaluating comprises: determining whether a corresponding amino acid at a 10^{th} motif position within the motif window matches one of the predetermined set of amino acids for the 10^{th} motif position, wherein the predetermined set of amino acids includes phenylalanine (F), leucine (L), and tyrosine (Y).

Embodiment 70. The method of any one of embodiments 59-69, wherein the evaluating comprises:
determining whether a corresponding amino acid at a 11^{th} motif position within the motif window is cysteine (C).

Embodiment 71. The method of any one of embodiments 59-70, wherein identifying the start position comprises: identifying the start position for CDR3 as an 11^{th} motif position of the 11 motif positions for the candidate start position of the plurality of candidate start positions having the highest score.

Embodiment 72. The method of embodiment 71 further comprising: identifying another start position for a framework region 4 (FWR4) based on the start position for the CDR3.

Embodiment 73. The method of any one of embodiments 1-72, wherein the selected region of interest is framework region 3 (FWR3).

Embodiment 74. The method of embodiment 73, wherein identifying the plurality of candidate start positions comprises: selecting a 40^{th} position before a previously identified start position for a complementarity-determining region 3 (CDR3) through a 34^{th} position before the previously identified start position for the CDR3 as the plurality of candidate start positions when the amino acid sequence is associated with a heavy chain; selecting a 35^{th} position before the previously identified start position for the CDR3 through a 28^{th} position before the previously identified start position for the CDR3 as the plurality of candidate start positions when the amino acid sequence is associated with a light chain; selecting a 36^{th} position before the previously identified start position for the CDR3 through a 33^{rd} position before the previously identified start position for the CDR3 as the plurality of candidate start positions when the amino acid sequence is associated with an alpha chain; and selecting a 38^{th} position before the previously identified start position for the CDR3 through the 35^{th} position before the previously identified start position for the CDR3 as the plurality of candidate start positions when the amino acid sequence is associated with a beta chain.

Embodiment 75. The method of embodiment 73 or embodiment 74, wherein the amino acid sequence is associated with a heavy chain, the motif window includes at least 10 motif positions, and wherein the generating the score comprises: evaluating seven motif positions of the at least 10 motif positions within the motif window for a corresponding candidate start position of the plurality of candidate start positions based on a predetermined set of amino acids corresponding to each motif position of the seven motif positions; and updating the score for the corresponding candidate start position for each motif position in the seven motif positions that matches a corresponding amino acid in the predetermined set of amino acids that corresponds to each motif position.

Embodiment 76. The method of embodiment 75, wherein identifying the start position comprises: identifying the start position for the FWR3 as a 1^{st} motif position of the motif window at the candidate start position of the plurality of candidate start positions having the highest score.

Embodiment 77. The method of embodiment 75 or embodiment 76, wherein the evaluating comprises: determining whether a corresponding amino acid at a 1^{st} motif position within the motif window matches one of the predetermined set of amino acids for the 1^{st} motif position, wherein the predetermined set of amino acids includes asparagine (N) and tyrosine (Y).

Embodiment 78. The method of any one of embodiments 75-77, wherein the evaluating comprises: determining whether a corresponding amino acid at a 2^{nd} motif position within the motif window is tyrosine (Y).

Embodiment 79. The method of any one of embodiments 75-78, wherein the evaluating comprises: determining whether a corresponding amino acid at a 3^{rd} motif position within the motif window matches one of the predetermined set of amino acids for the 3^{rd} motif position, wherein the predetermined set of amino acids includes alanine (A) and asparagine (N).

Embodiment 80. The method of any one of embodiments 75-79, wherein the evaluating comprises: determining whether a corresponding amino acid at a 6^{th} motif position within the motif window matches one of the predetermined set of amino acids for the 6^{th} motif position, wherein the predetermined set of amino acids includes phenylalanine (F) and leucine (L).

Embodiment 81. The method of any one of embodiments 75-80, wherein the evaluating comprises: determining whether a corresponding amino acid at a 7^{th} motif position within the motif window matches one of the predetermined set of amino acids for the 7^{th} motif position, wherein the predetermined set of amino acids includes lysine (K), glutamine (Q), and arginine (R).

Embodiment 82. The method of any one of embodiments 75-81, wherein the evaluating comprises: determining whether a corresponding amino acid at a 9^{th} motif position within the motif window matches one of the predetermined set of amino acids for the 9^{th} motif position, wherein the predetermined set of amino acids includes lysine (K) and arginine (R).

Embodiment 83. The method of any one of embodiments 75-82, wherein the evaluating comprises: determining whether a corresponding amino acid at a 10^{th} motif position within the motif window matches one of the predetermined set of amino acids for the 10^{th} motif position, wherein the predetermined set of amino acids includes alanine (A), phenylalanine (F), valine (V), and leucine (L).

Embodiment 84. The method of embodiment 73, wherein the amino acid sequence is associated with a light chain, the motif window includes at least 8 motif positions, and wherein the generating the score comprises: evaluating five motif positions of the at least 8 motif positions within the motif window for a corresponding candidate start position of the plurality of candidate start positions based on a predetermined set of amino acids corresponding to each motif position of the five motif positions; and updating the score for the corresponding candidate start position for each motif position in the five motif positions that matches a corresponding amino acid in the predetermined set of amino acids that corresponds to each motif position.

Embodiment 85. The method of embodiment 84, wherein identifying the start position comprises: identifying the start position for the FWR3 as a 1^{st} motif position of the motif window at the candidate start position of the plurality of candidate start positions having the highest score.

Embodiment 86. The method of embodiment 84 or embodiment 85, wherein the evaluating comprises: determining whether a corresponding amino acid at a 1^{st} motif position within the motif window is glycine (G).

Embodiment 87. The method of any one of embodiments 84-86, wherein the evaluating comprises: determining whether a corresponding amino acid at a 3^{rd} motif position within the motif window is proline (P).

Embodiment 88. The method of any one of embodiments 84-87, wherein the evaluating comprises: determining whether a corresponding amino acid at a 5^{th} motif position within the motif window is arginine (R).

Embodiment 89. The method of any one of embodiments 84-88, wherein the evaluating comprises: determining whether a corresponding amino acid at a 6^{th} motif position within the motif window is phenylalanine (F).

Embodiment 90. The method of any one of embodiments 84-89, wherein the evaluating comprises: determining whether a corresponding amino acid at an 8^{th} motif position within the motif window is glycine (G).

Embodiment 91. The method of embodiment 73, wherein the amino acid sequence is associated with an alpha chain, the motif window includes at least 12 motif positions, and wherein the generating the score comprises: evaluating 11 motif positions of the at least 12 motif positions within the motif window for a corresponding candidate start position of the plurality of candidate start positions based on a predetermined set of amino acids corresponding to each motif position of the 11 motif positions; and updating the score for the corresponding candidate start position for each motif position in the 11 motif positions that matches a corresponding amino acid in the predetermined set of amino acids that corresponds to each motif position.

Embodiment 92. The method of embodiment 91, wherein identifying the start position comprises: identifying the start position for the FWR3 as a one position before a 1^{st} motif position of the motif window at the candidate start position of the plurality of candidate start positions having the highest score.

Embodiment 93. The method of embodiment 91 or embodiment 92, wherein the evaluating comprises: determining whether a corresponding amino acid at a 1^{st} motif position within the motif window matches one of the predetermined set of amino acids for the 1^{st} motif position, wherein the predetermined set of amino acids includes glutamic acid (E), lysine (K), asparagine (N), and valine (V).

Embodiment 94. The method of any one of embodiments 91-93, wherein the evaluating comprises: determining whether a corresponding amino acid at a 2^{nd} motif position within the motif window matches one of the predetermined set of amino acids for the 2^{nd} motif position, wherein the predetermined set of amino acids includes alanine (A), glutamic acid (E), lysine (K), and threonine (T).

Embodiment 95. The method of any one of embodiments 91-94, wherein the evaluating comprises: determining whether a corresponding amino acid at a 3^{rd} motif position within the motif window matches one of the predetermined set of amino acids for the 3^{rd} motif position, wherein the predetermined set of amino acids includes glutamic acid (E) and serine (S).

Embodiment 96. The method of any one of embodiments 91-95, wherein the evaluating comprises: determining whether a corresponding amino acid at a 4^{th} motif position within the motif window matches one of the predetermined set of amino acids for the 4^{th} motif position, wherein the predetermined set of amino acids includes aspartic acid (D), asparagine (N), and serine (S).

Embodiment 97. The method of any one of embodiments 91-96, wherein the evaluating comprises: determining whether a corresponding amino acid at a 5^{th} motif position within the motif window is asparagine (N).

Embodiment 98. The method of any one of embodiments 91-97, wherein the evaluating comprises: determining whether a corresponding amino acid at a 6^{th} motif position within the motif window matches one of the predetermined set of amino acids for the 6^{th} motif position, wherein the predetermined set of amino acids includes glycine (G), methionine (M), and arginine (R).

Embodiment 99. The method of any one of embodiments 91-98, wherein the evaluating comprises: determining whether a corresponding amino acid at a 7^{th} motif position within the motif window matches one of the predetermined set of amino acids for the 7^{th} motif position, wherein the predetermined set of amino acids includes alanine (A), phenylalanine (F), isoleucine (I), and tyrosine (Y).

Embodiment 100. The method of any one of embodiments 91-99, wherein the evaluating comprises: determining whether a corresponding amino acid at an 8^{th} motif position within the motif window matches one of the predetermined set of amino acids for the 8^{th} motif position, wherein the predetermined set of amino acids includes serine (S) and threonine (T).

Embodiment 101. The method of any one of embodiments 91-100, wherein the evaluating comprises: determining whether a corresponding amino acid at a 9^{th} motif position within the motif window matches one of the predetermined set of amino acids for the 9^{th} motif position, wherein the predetermined set of amino acids includes alanine (A) and valine (V).

Embodiment 102. The method of any one of embodiments 91-101, wherein the evaluating comprises: determining whether a corresponding amino acid at a 10^{th} motif position within the motif window matches one of the predetermined set of amino acids for the 10^{th} motif position, wherein the predetermined set of amino acids includes glutamic acid (E) and threonine (T).

Embodiment 103. The method of any one of embodiments 91-102, wherein the evaluating comprises: determining whether a corresponding amino acid at a 12^{th} motif position within the motif window matches one of the predetermined set of amino acids for the 12^{th} motif position, wherein the predetermined set of amino acids includes aspartic acid (D) and asparagine (N).

Embodiment 104. The method of embodiment 73, wherein the amino acid sequence is associated with a beta chain, the motif window includes five motif positions, and wherein the generating the score comprises: evaluating the five motif positions within the motif window for a corresponding candidate start position of the plurality of candidate start positions based on a predetermined set of amino acids corresponding to each motif position of the five motif positions; and updating the score for the corresponding candidate start position for each motif position in the five motif positions that matches a corresponding amino acid in the predetermined set of amino acids that corresponds to each motif position.

Embodiment 105. The method of embodiment 104, wherein identifying the start position comprises: identifying the start position for the FWR3 as two positions before a 1^{st} motif position of the motif window at the candidate start position of the plurality of candidate start positions having the highest score.

Embodiment 106. The method of embodiment 104 or embodiment 105, wherein the evaluating comprises: determining whether a corresponding amino acid at a 1^{st} motif position within the motif window matches one of the predetermined set of amino acids for the 1^{st} motif position, wherein the predetermined set of amino acids includes aspartic acid (D), glutamic acid (E), and lysine (K).

Embodiment 107. The method of any one of embodiments 104-106, wherein the evaluating comprises: determining whether a corresponding amino acid at a 2^{nd} motif position within the motif window matches one of the predetermined set of amino acids for the 2^{nd} motif position, wherein the predetermined set of amino acids includes glycine (G), glutamine (Q), and serine (S).

Embodiment 108. The method of any one of embodiments 104-106, wherein the evaluating comprises: determining whether a corresponding amino acid at a 3^{rd} motif position within the motif window matches one of the predetermined set of amino acids for the 3^{rd} motif position, wherein the predetermined set of amino acids includes aspartic acid (D), glutamic acid (E), glycine (G), and serine (S).

Embodiment 109. The method of any one of embodiments 104-106, wherein the evaluating comprises: determining whether a corresponding amino acid at a 4^{th} motif position within the motif window matches one of the predetermined set of amino acids for the 4^{th} motif position, wherein the predetermined set of amino acids includes isoleucine (I), leucine (L), methionine (M), and valine (V).

Embodiment 110. The method of any one of embodiments 104-106, wherein the evaluating comprises: determining whether a corresponding amino acid at a 5^{th} motif position within the motif window matches one of the predetermined set of amino acids for the 5^{th} motif position, wherein the predetermined set of amino acids includes proline (P) and serine (S).

Embodiment 111. The method of any one of embodiments 1-110, further comprising: determining whether the start position identified for the selected region of interest meets a set of validation criteria; and providing an indication that the start position is not valid in response to a determination that the start position does not meet the set of validation criteria.

Embodiment 112. The method of any one of embodiments 1-111, further comprising: detecting a presence of a set of indels within the amino acid sequence; and updating the start position based on the set of indels.

Embodiment 113. The method of any one of embodiments 1-112, further comprising: generating a sequence output that includes an identification of a region sequence for the selected region of interest, the start position for the selected region of interest, and a stop position for the selected region of interest, wherein the identification has an amino acid format.

Embodiment 114. The method of any one of embodiments 1-113, further comprising: generating a sequence output that includes an identification of a region sequence for the selected region of interest, the start position for the selected region of interest, and a stop position for the selected region of interest, wherein the identification has a nucleotide format.

Embodiment 115. A method for identifying framework regions and complementarity-determining regions in an amino acid sequence, the method comprising: receiving the amino acid sequence and a chain type for the amino acid sequence; identifying a first start position for FWR1 within the amino acid sequence using an FWR1 motif, identifying a second start position for CDR1 within the amino acid sequence using the chain type, the first start position, and a CDR1 motif, identifying a third start position for FWR2 within the amino acid sequence using the chain type and an FWR2 motif, identifying a fourth start position for CDR2 within the amino acid sequence using the chain type and the third start position; identifying a fifth start position for CDR3 within the amino acid sequence using a CDR3 motif, identifying a sixth start position for FWR3 within the amino acid sequence using the fifth start position and a FWR3 motif selected based on the chain type; and identifying a seventh start position for FWR4 based on the fifth start position; validating the first start position, the second start position, the third start position, the fourth start position, the fifth start position, and the sixth start position; and generating a sequence output that identifies sequences for one or more of the framework regions and complementarity-determining regions determined to have valid start positions.

Embodiment 116. The method of embodiment 115, further comprising: detecting a presence of a set of indels within the amino acid sequence; and updating at least one of the first start position, the second start position, the third start position, the fourth start position, the fifth start position, the sixth start position, or the seventh start position based on the set of indels.

Embodiment 117. A system for identifying framework regions and complementarity-determining regions in an amino acid sequence, the system comprising a data source configured to obtain the amino acid sequence generated from a sample, wherein the amino acid sequence is for a chain of an immune cell receptor that is associated with an individual immune cell in the sample; and a processor configured to receive the amino acid sequence from the data source and further configured to: identify a plurality of candidate start positions within the amino acid sequence for a start position for a selected region of interest; generate a score for each candidate start position of the plurality of candidate start positions via analysis of a motif window that begins at each candidate start position; and identify the start position for the selected region of interest based on a candidate start position of the plurality of candidate start positions having a highest score.

Embodiment 118. The system of embodiment 117, wherein the selected region of interest is either a framework region or a complementarity-determining region and wherein the chain has a chain type selected from a group consisting of a heavy chain, a light chain, an alpha chain, and a beta chain.

Embodiment 119. The system of embodiment 117 or embodiment 118, wherein: the processor is further configured to evaluate a set of motif positions within the motif window for a corresponding candidate start position of the plurality of candidate start positions based on a predetermined set of amino acids corresponding to each motif position of the set of motif positions; and the processor is further configured to update the score for the corresponding candidate start position for each motif position in the set of motif positions that matches a corresponding amino acid in the predetermined set of amino acids that corresponds to each motif position.

Embodiment 120. The system of embodiment 119, wherein the processor is further configured to weigh each motif position of the set of motif positions differently from a least one other motif position of the set of motif positions.

Embodiment 121. The system of embodiment 119 or embodiment 120, wherein: the processor is further configured to determine whether the start position identified for the selected region of interest meets a set of validation criteria; and the processor is further configured to provide an indication that the start position is not valid in response to a determination that the start position does not meet the set of validation criteria.

Embodiment 122. The system of any one of embodiments 117-121, wherein: the processor is further configured to detect a presence of a set of indels within the amino acid sequence; and the processor is further configured to update the start position based on the set of indels.

Embodiment 123. The system of any one of embodiments 117-122, wherein the processor is further configured to generate a sequence output that includes an identification of a region sequence for the selected region of interest, the start position for the selected region of interest, and a stop position for the selected region of interest, wherein the identification has an amino acid format.

Embodiment 124. The system of any one of embodiments 117-122, wherein the processor is further configured to generate a sequence output that includes an identification of a region sequence for the selected region of interest, the start position for the selected region of interest, and a stop position for the selected region of interest, wherein the identification has a nucleotide format.

Embodiment 125. A non-transitory computer-readable medium in which a program is stored, the program being configured for causing a computer to perform a method for identifying framework regions and complementarity-determining regions in an amino acid sequence, the method comprising: receiving the amino acid sequence; identifying a plurality of candidate start positions within the amino acid sequence for a start position for a selected region of interest; generating a score for each candidate start position of the plurality of candidate start positions via analysis of a motif window that begins at each candidate start position; and identifying the start position for the selected region of interest based on a candidate start position of the plurality of candidate start positions having a highest score.

Embodiment 126. The non-transitory computer-readable medium of embodiment 125, wherein the selected region of interest is either a framework region or a complementarity-determining region.

Embodiment 127. The non-transitory computer-readable medium of embodiment 125 or embodiment 126, wherein the amino acid sequence is for a chain of an immune cell receptor.

Embodiment 128. The non-transitory computer-readable medium of any one of embodiments 125-127, wherein the program being configured to cause the computer to generate the score includes the program being configured to cause the computer to: evaluate a set of motif positions within the motif window for a corresponding candidate start position of the plurality of candidate start positions based on a predetermined set of amino acids corresponding to each motif position of the set of motif positions; and update the score for the corresponding candidate start position for each motif position in the set of motif positions that matches a corresponding amino acid in the predetermined set of amino acids that corresponds to each motif position.

Embodiment 129. The non-transitory computer-readable medium of embodiment 128, wherein the program is further configured to cause the computer to weigh each motif position of the set of motif positions differently from a least one other motif position of the set of motif positions.

Embodiment 130. The non-transitory computer-readable medium of embodiment any one of embodiments 125-129, wherein the program is further configured to cause the computer to: determine whether the start position identified for the selected region of interest meets a set of validation criteria; and provide an indication that the start position is not valid in response to a determination that the start position does not meet the set of validation criteria.

Embodiment 131. The non-transitory computer-readable medium of embodiment any one of embodiments 125-130, wherein the program is further configured to cause the computer to: detect a presence of a set of indels within the amino acid sequence; and update the start position based on the set of indels.

Embodiment 132. The non-transitory computer-readable medium of embodiment any one of embodiments 125-131, wherein the program is further configured to cause the computer to: generate a sequence output that includes an identification of a region sequence for the selected region of interest, the start position for the selected region of interest, and a stop position for the selected region of interest, wherein the identification has an amino acid format.

Embodiment 133. The non-transitory computer-readable medium of embodiment any one of embodiments 125-132, wherein the program is further configured to cause the computer to: generate a sequence output that includes an identification of a region sequence for the selected region of interest, the start position for the selected region of interest, and a stop position for the selected region of interest, wherein the identification has a nucleotide format.

Embodiment 134. A non-transitory computer-readable medium in which a program is stored, the program being configured for causing a computer to perform a method for identifying framework regions and complementarity-determining regions in an amino acid sequence, the method comprising: receiving the amino acid sequence and a chain type for the amino acid sequence; identifying a first start position for FWR1 within the amino acid sequence using an FWR1 motif, identifying a second start position for CDR1 within the amino acid sequence using the chain type, the first start position, and a CDR1 motif, identifying a third start position for FWR2 within the amino acid sequence using the chain type and an FWR2 motif, identifying a fourth start position for CDR2 within the amino acid sequence using the chain type and the third start position; identifying a fifth start position for CDR3 within the amino acid sequence using a CDR3 motif, identifying a sixth start position for FWR3 within the amino acid sequence using the fifth start position and a FWR3 motif selected based on the chain type; and identifying a seventh start position for FWR4 based on the fifth start position; validating the first start position, the second start position, the third start position, the fourth start position, the fifth start position, and the sixth start position; and generating a sequence output that identifies sequences for one or more of the framework regions and complementarity-determining regions determined to have valid start positions.

Embodiment 135. The non-transitory computer-readable medium of embodiment 134, wherein the program is further configured to cause the computer to: detect a presence of a set of indels within the amino acid sequence; and update at least one of the first start position, the second start position, the third start position, the fourth start position, the fifth start position, the sixth start position, or the seventh start position based on the set of indels.

Embodiment 136. A system for identifying framework regions and complementarity-determining regions in an amino acid sequence, the system comprising: a data source configured to obtain the amino acid sequence, and a chain type for the amino acid sequence, generated from a sample, wherein the amino acid sequence is for a chain of an immune cell receptor that is associated with an individual immune cell in the sample; and a processor configured to receive the amino acid sequence, and the chain type for the amino acid sequence, from the data source and further configured to: identify a first start position for FWR1 within the amino acid sequence using an FWR1 motif, identify a second start position for CDR1 within the amino acid sequence using the chain type, the first start position, and a CDR1 motif, identify a third start position for FWR2 within the amino acid sequence using the chain type and an FWR2 motif, identify a fourth start position for CDR2 within the amino acid sequence using the chain type and the third start position; identify a fifth start position for CDR3 within the amino acid sequence using a CDR3 motif, identify a sixth start position for FWR3 within the amino acid sequence using the fifth start position and a FWR3 motif selected based on the chain type; and identify a seventh start position for FWR4 based on the fifth start position; validate the first start position, the second start position, the third start position, the fourth start position, the fifth start position, and the sixth start position; and generate a sequence output that identifies sequences for one or more of the framework regions and complementarity-determining regions determined to have valid start positions.

Embodiment 137. The system of embodiment 136, wherein the processor is further configured to: detect a presence of a set of indels within the amino acid sequence; and update at least one of the first start position, the second start position, the third start position, the fourth start position, the fifth start position, the sixth start position, or the seventh start position based on the set of indels.

1. A method for identifying framework regions and complementarity-determining regions in an amino acid sequence, the method comprising:
receiving the amino acid sequence;
identifying a plurality of candidate start positions within the amino acid sequence for a start position for a selected region of interest;
generating a score for each candidate start position of the plurality of candidate start positions via analysis of a motif window that begins at each candidate start position; and
identifying the start position for the selected region of interest based on a candidate start position of the plurality of candidate start positions having a highest score.

2. The method of claim 1, wherein the selected region of interest is either a framework region or a complementarity-determining region; and/or
wherein generating the score comprises:
evaluating a set of motif positions within the motif window for a corresponding candidate start position of the plurality of candidate start positions based on a predetermined set of amino acids corresponding to each motif position of the set of motif positions; and
updating the score for the corresponding candidate start position for each motif position in the set of motif positions that matches a corresponding amino acid in the predetermined set of amino acids that corresponds to each motif position.

3. (canceled)

4. The method of claim 32, wherein each motif position of the set of motif positions is weighted differently from at least one other motif position of the set of motif positions.

5. The method of claim 1, wherein the selected region of interest is complementarity-determining region 3 (CDR3), framework region 1 (FWR1), complementarity-determining region 1 (CDR1), framework region 2 (FWR2), complementarity-determining region 2 (CDR2), or framework region 3 (FWR3).

6. The method of claim 5, wherein:
the selected region of interest is CDR3, and wherein identifying the plurality of candidate start positions comprises:
selecting all positions in the amino acid sequence as the plurality of candidate start positions, and/or
selecting all positions in the amino acid sequence after a previously identified start position for one of FWR1, CDR1, FWR2, or CDR2;
or
the selected region of interest is FWR1, and wherein the identifying the plurality of candidate start positions comprises:
selecting 50 positions at a beginning of the amino acid sequence as the plurality of candidate start positions;
or
wherein the selected region of interest is CDR1, and wherein identifying the plurality of candidate start positions comprises:
selecting 9 positions that begin 19 positions after a previously identified start position for a framework region 1 (FWR1) as the plurality of candidate start positions;
or
wherein the selected region of interest is FWR2, and wherein identifying the plurality of candidate start positions comprises:
selecting 23 positions beginning with a 40^{th} Position of the amino acid sequence as the plurality of candidate start positions when the amino acid sequence is associated with a heavy chain; and
selecting 34 positions beginning with a 40^{th} position of the amino acid sequence as the plurality of candidate start positions when the amino acid sequence is associated with one of a lambda light chain, a kappa light chain, an alpha chain, or a beta chain;
or
wherein the selected region of interest is CDR2, and wherein identifying the plurality of candidate start positions comprises:
selecting six positions after a previously identified start position for framework region 2 (FWR2) as the plurality of candidate start positions when the amino acid sequence is associated with a heavy chain; and
selecting three positions after the previously identified start position for the FWR2 as the plurality of candidate start positions when the amino acid sequence is associated with one of an alpha chain;
or
wherein the selected region of interest is FWR3, and wherein identifying the plurality of candidate start positions comprises:
selecting a 40^{th} position before a previously identified start position for a complementarity-determining region 3 (CDR3) through a 34^{th} position before the previously identified start position for the CDR3 as the plurality of candidate start positions when the amino acid sequence is associated with a heavy chain;
selecting a 35^{th} position before the previously identified start position for the CDR3 through a 28^{th} position before the previously identified start position for the CDR3 as the plurality of candidate start positions when the amino acid sequence is associated with a light chain;
selecting a 36^{th} position before the previously identified start position for the CDR3 through a 33^{rd} position before the previously identified start position for the CDR3 as the plurality of candidate start positions when the amino acid sequence is associated with an alpha chain; and
selecting a 38^{th} position before the previously identified start position for the CDR3 through the 35^{th} position before the previously identified start position for the CDR3 as the plurality of candidate start positions when the amino acid sequence is associated with a beta chain.

7. (canceled)

8. The method of claim 5, wherein:
the selected region of interest is CDR3, and wherein the motif window includes at least 11 motif positions and wherein the generating the score comprises:
evaluating the 11 motif positions within the motif window for a corresponding candidate start position of the plurality of candidate start positions based on a predetermined set of amino acids corresponding to each motif position of the 11 motif positions; and
updating the score for the corresponding candidate start position for each motif position in the 11 motif positions that matches a corresponding amino acid in the predetermined set of amino acids that corresponds to each motif position;
or
the selected region of interest is FWR1, and wherein the motif window includes at least 23 motif positions and wherein generating the score comprises:
evaluating six motif positions of the at least 23 motif positions within the motif window for a corresponding candidate start position of the plurality of candidate start positions based on a predetermined set of amino acids corresponding to each motif position of the six motif positions; and
updating the score for the corresponding candidate start position for each motif position in the six motif positions that matches a corresponding amino acid in the predetermined set of amino acids that corresponds to each motif position;
or
wherein the selected region of interest is CDR1, and wherein the motif window includes at least nine motif positions and wherein the generating the score comprises:
evaluating six motif positions of the at least nine motif positions within the motif window for a corresponding candidate start position of the plurality of candidate start positions based on a predetermined set of amino acids corresponding to each motif position of the six motif positions; and
updating the score for the corresponding candidate start position for each motif position in the six motif positions that matches a corresponding amino acid in the predetermined set of amino acids that corresponds to each motif position;
or
wherein the selected region of interest is FWR2, and wherein the motif window includes at least 11 motif positions and wherein the generating the score comprises:
evaluating nine motif positions of the at least 11 motif positions within the motif window for a corresponding candidate start position of the plurality of candidate start positions based on a predetermined set of amino acids corresponding to each motif position of the nine motif positions; and
updating the score for the corresponding candidate start position for each motif position in the nine motif positions that matches a corresponding amino acid in the predetermined set of amino acids that corresponds to each motif position;
or
wherein the selected region of interest is CDR2, and wherein the motif window includes at least five motif positions and wherein the generating the score comprises:
evaluating the five motif positions within the motif window for a corresponding candidate start position of the plurality of candidate start positions based on a predetermined set of amino acids corresponding to each motif position of the five motif positions; and
updating the score for the corresponding candidate start position for each motif position in the five motif positions that matches a corresponding amino acid in the predetermined set of amino acids that corresponds to each motif position.

9. The method of claim 8, wherein the selected region of interest is CDR3, and wherein the evaluating comprises:
determining whether a corresponding amino acid at a 1^{st} motif position within the motif window matches one of the predetermined set of amino acids for the 1^{st} motif position, wherein the predetermined set of amino acids includes alanine (A), leucine (L), and valine (V); and/or
determining whether a corresponding amino acid at a 2^{nd} motif position within the motif window matches one of the predetermined set of amino acids for the 2^{nd} motif position, wherein the predetermined set of amino acids includes glutamic acid (E) glutamine (Q), and threonine (T); and/or
determining whether a corresponding amino acid at a 3rd motif position within the motif window matches one of the predetermined set of amino acids for the 3^{rd} motif position, wherein the predetermined set of amino acids includes alanine (A), proline (P), and serine (S); and/or
determining whether a corresponding amino acid at a 4^{th} motif position within the motif window matches one of the predetermined set of amino acids for the 4^{th} motif position, wherein the predetermined set of amino acids includes glutamic acid (E), glycine (G), or serine (S); and/or
determining whether a corresponding amino acid at a 5^{th} motif position within the motif window matches one of the predetermined set of amino acids for the 5^{th} motif position, wherein the predetermined set of amino acids includes aspartic acid (D) and glutamine (Q); and/or
determining whether a corresponding amino acid at a 6^{th} motif position within the motif window matches one of the predetermined set of amino acids for the 6^{th} motif position, wherein the predetermined set of amino acids includes alanine (A), serine (S), and threonine (T); and/or
determining whether a corresponding amino acid at a 7^{th} motif position within the motif window matches one of the predetermined set of amino acids for the 7^{th} motif position, wherein the predetermined set of amino acids includes alanine (A), serine (S), and glycine (G); and/or
determining whether a corresponding amino acid at an 8^{th} motif position within the motif window matches one of the predetermined set of amino acids for the 8^{th} motif position, wherein the predetermined set of amino acids includes leucine (L), threonine (T), and valine (V); and/or
determining whether a corresponding amino acid at a 9^{th} motif position within the motif window is tyrosine (Y); and/or
determining whether a corresponding amino acid at a 10^{th} motif position within the motif window matches one of the predetermined set of amino acids for the 10^{th} motif position, wherein the predetermined set of amino acids includes phenylalanine (F), leucine (L), and tyrosine (Y); and/or
determining whether a corresponding amino acid at a 11^{th} motif position within the motif window is cysteine (C).

10-19. (canceled)

20. The method of claim 8, wherein:
the selected region of interest is CDR3, and wherein identifying the start position comprises:
identifying the start position for CDR3 as an 11^{th} motif position of the 11 motif positions for the candidate start position of the plurality of candidate start positions having the highest score; and/or further comprising:
identifying another start position for a framework region 4 (FWR4) based on the start position for the CDR3;
or
the selected region of interest is FWR1, and wherein identifying the start position comprises:
identifying the start position for the FWR1 as a 1 motif position of the motif window at the candidate start position of the plurality of candidate start positions having the highest score;
or
the selected region of interest is CDR1, and wherein identifying the start position comprises:
identifying the start position for the CDR1 as a 5^{th} position after the candidate start position of the plurality of candidate start positions having the highest score when the amino acid sequence is associated with one of a lambda light chain or a kappa light chain, and identifying the start position for the CDR1 as an 8^{th} position after the candidate start position of the plurality of candidate start positions having the highest score when the amino acid sequence is associated with one of an alpha chain, a beta chain, or a heavy chain;
or
the selected region of interest is FWR2, and wherein identifying the start position comprises:
identifying the start position for the FWR2 as a 1^{st} motif position of the motif window at the candidate start position of the plurality of candidate start positions having the highest score when the amino acid sequence is associated with a heavy chain, identifying the start position for the FWR2 as a 2^{nd} motif position of the motif window at the candidate start position of the plurality of candidate start positions having the highest score when the amino acid sequence is associated with a light chain, and identifying the start position for the FWR2 as one position before a 1^{st} motif position of the motif window at the candidate start position of the plurality of candidate start positions having the highest score when the amino acid sequence is associated with an alpha chain or a beta chain;
or
the selected region of interest is CDR2, and wherein identifying the start position comprises:
identifying the start position for the CDR2 as a 7^{th} position after a 1^{st} motif position of the motif window at the candidate start position of the plurality of candidate start positions having the highest score when the amino acid sequence is associated with a heavy chain, and identifying the start position for the CDR2 as a 6^{th} position after 1^{st} motif position of the motif window at the candidate start position of the plurality of candidate start positions having the highest score when the amino acid sequence is associated with an alpha chain.

21-25. (canceled)

26. The method of claim 8, wherein the selected region of interest is FWR1, and wherein the evaluating comprises:
determining whether a corresponding amino acid at a 1^{st} motif position within the motif window matches one of the predetermined set of amino acids for the 1^{st} motif position, wherein the predetermined set of amino acids includes glutamine (Q), aspartic acid (D), glutamic acid (E), lysine (K), or glycine (G); and/or
determining whether a corresponding amino acid at a 1^{st} motif position within the motif window is cysteine (C), and updating positions of the motif window in response to a determination that the 1^{st} motif position within the motif window is cysteine; and/or
determining whether a corresponding amino acid at a 2^{nd} motif position within the motif window matches one of the predetermined set of amino acids for the 2^{nd} motif position, wherein the predetermined set of amino acids includes alanine (A), isoleucine (I), glutamine (Q), and valine (V); and/or
determining whether a corresponding amino acid at a 4^{th} motif position within the motif window matches one of the predetermined set of amino acids for the 4^{th} motif position, wherein the predetermined set of amino acids includes leucine (L), methionine (M), and valine (V); and/or
determining whether a corresponding amino acid at a 6^{th} motif position within the motif window matches one of the predetermined set of amino acids for the 6^{th} motif position, wherein the predetermined set of amino acids includes glutamic acid (E) and glutamine (Q); and/or
determining whether a corresponding amino acid at a 22^{nd} motif position within the motif window is cysteine (C); and/or
determining whether a corresponding amino acid at a 23^{rd} motif position within the motif window is cysteine (C).

27-37. (canceled)

38. The method of claim 8, wherein the selected region of interest is CDR1, and wherein the evaluating comprises:
determining whether a corresponding amino acid at a 1^{st} motif position within the motif window is valine (V); and/or
determining whether a corresponding amino acid at a 2^{nd} motif position within the motif window is threonine (T); and/or
determining whether a corresponding amino acid at a 3^{rd} motif position within the motif window matches one of the predetermined set of amino acids for the 3^{rd} motif position, wherein the predetermined set of amino acids includes isoleucine (I), leucine (L), methionine (M), and valine (V); and/or
determining whether a corresponding amino acid at a 4^{th} motif position within the motif window matches one of the predetermined set of amino acids for the 4^{th} motif position, wherein the predetermined set of amino acids includes arginine (R), serine (S), and threonine (T); and/or
determining whether a corresponding amino acid at a 5^{th} motif position within the motif window is cysteine (C); and/or
determining whether a corresponding amino acid at an 8^{th} motif position within the motif window matches one of the predetermined set of amino acids for the 8^{th} motif position, wherein the predetermined set of amino acids includes isoleucine (I), serine (S), and aspartic acid (A).

39-47. (canceled)

48. The method of claim 468, wherein the selected region of interest is FWR2, and wherein the evaluating comprises:
determining whether a corresponding amino acid at a 1^{st} motif position within the motif window matches one of the predetermined set of amino acids for the 1^{st} motif position, wherein the predetermined set of amino acids includes phenylalanine (F), leucine (L), methionine (M), and valine (V); and/or
determining whether a corresponding amino acid at a 2^{nd} motif position within the motif window is tyrosine (Y); and/or
determining whether a corresponding amino acid at a 3^{rd} motif position within the motif window is tryptophan (W); and/or
determining whether a corresponding amino acid at a 4^{th} motif position within the motif window is tyrosine (Y); and/or
determining whether a corresponding amino acid at a 5^{th} motif position within the motif window is arginine (R); and/or
determining whether a corresponding amino acid at a 6^{th} motif position within the motif window is glutamine (Q); and/or
determining whether a corresponding amino acid at a 9^{th} motif position within the motif window is glycine (G); and/or
determining whether a corresponding amino acid at a 10^{th} motif position within the motif window matches one of the predetermined set of amino acids for the 10^{th} motif position, wherein the predetermined set of amino acids include lysine (K) or glutamine (Q); and/or
determining whether a corresponding amino acid at a 11^{th} motif position within the motif window matches one of the predetermined set of amino acids for the 11^{th} motif position, wherein the predetermined set of amino acids include alanine (A), glycine (G), and lysine (K).

49-56. (canceled)

57. The method of claim 8, wherein the selected region of interest is FWR2, and further comprising:
identifying another start position for complementarity-determining region 2 (CDR2) as a 15^{th} position after the start position for FWR2 when the amino acid sequence is associated with a light chain; and/or
identifying another start position for complementarity-determining region 2 (CDR2) as a 17^{th} position after the start position for FWR2 when the amino acid sequence is associated with a beta chain.

58-62. (canceled)

63. The method of claim 8, wherein the selected region of interest is CDR2, and wherein:
the amino acid sequence is associated with a heavy chain and wherein the evaluating comprises:
determining whether a corresponding amino acid at a 1^{st} motif position within the motif window is leucine (L); and/or
determining whether a corresponding amino acid at a 2^{nd} motif position within the motif window is glutamic acid (E); and/or
determining whether a corresponding amino acid at a 3^{rd} motif position within the motif window is tryptophan (W); and/or
determining whether a corresponding amino acid at a 4^{th} motif position within the motif window matches one of the predetermined set of amino acids for the 4^{th} motif position, wherein the predetermined set of amino acids include isoleucine (I), leucine (L), methionine (M), and valine (V), and/or
determining whether a corresponding amino acid at a 5^{th} motif position within the motif window matches one of the predetermined set of amino acids for the 5^{th} motif position, wherein the predetermined set of amino acids include alanine (A), glycine (G), and serine (S);
or
the amino acid sequence is associated with an alpha chain and wherein the evaluating comprise:
determining whether a corresponding amino acid at a 1^{st} motif position within the motif window matches one of the predetermined set of amino acids for the 1^{st} motif position, wherein the predetermined set of amino acids include leucine (L), and proline (P); and/or
determining whether a corresponding amino acid at a 2^{nd} motif position within the motif window matches one of the predetermined set of amino acids for the 2^{nd} motif position, wherein the predetermined set of amino acids includes glutamic acid (E), isoleucine (I), glutamine (Q), threonine (T), and valine (V); and/or
determining whether a corresponding amino acid at a 3^{rd} motif position within the motif window matches one of the predetermined set of amino acids for the 3^{rd} motif position, wherein the predetermined set of amino acids includes phenylalanine (F) and leucine (L); and/or
determining whether a corresponding amino acid at a 4^{th} motif position within the motif window is leucine (L); and/or
determining whether a corresponding amino acid at a 5^{th} motif position within the motif window matches one of the predetermined set of amino acids for the 5^{th} motif position, wherein the predetermined set of amino acids includes isoleucine (I) and leucine (L).

64-74. (canceled)

75. The method of claim 5, wherein the selected region of interest is FWR3, and wherein:
the amino acid sequence is associated with a heavy chain, the motif window includes at least 10 motif positions, and wherein the generating the score comprises:
evaluating seven motif positions of the at least 10 motif positions within the motif window for a corresponding candidate start position of the plurality of candidate start positions based on a predetermined set of amino acids corresponding to each motif position of the seven motif positions; and
updating the score for the corresponding candidate start position for each motif position in the seven motif positions that matches a corresponding amino acid in the predetermined set of amino acids that corresponds to each motif position,
or
the amino acid sequence is associated with a light chain, the motif window includes at least 8 motif positions, and wherein the generating the score comprises:
evaluating five motif positions of the at least 8 motif positions within the motif window for a corresponding candidate start position of the plurality of candidate start positions based on a predetermined set of amino acids corresponding to each motif position of the five motif positions; and
updating the score for the corresponding candidate start position for each motif position in the five motif positions that matches a corresponding amino acid in the predetermined set of amino acids that corresponds to each motif position;
or
the amino acid sequence is associated with a alpha chain, the motif window includes at least 12 motif positions, and wherein the generating the score comprises:
evaluating 11 motif positions of the at least 12 motif positions within the motif window for a corresponding candidate start position of the plurality of candidate start positions based on a predetermined set of amino acids corresponding to each motif position of the five motif positions; and
updating the score for the corresponding candidate start position for each motif position in the five motif positions that matches a corresponding amino acid in the predetermined set of amino acids that corresponds to each motif position;
or
the amino acid sequence is associated with a beta chain, the motif window includes five motif positions, and wherein the generating the score comprises:
evaluating the five motif positions within the motif window for a corresponding candidate start position of the plurality of candidate start positions based on a predetermined set of amino acids corresponding to each motif position of the five motif positions; and
updating the score for the corresponding candidate start position for each motif position in the five motif positions that matches a corresponding amino acid in the predetermined set of amino acids that corresponds to each motif position.

76. The method of claim 75, wherein:
the amino acid sequence is associated with a heavy chain, and the motif window includes at least 10 motif positions, and wherein identifying the start position comprises identifying the start position for the FWR3 as a 1^{st} motif position of the motif window at the candidate start position of the plurality of candidate start positions having the highest score; or
the amino sequence is associated with a light chain, the motif window includes at least 8 motif positions, and wherein identifying the start position comprises identifying the start position for the FWR3 as a 1^{st} motif position of the motif window at the candidate start position of the plurality of candidate start positions having the highest score; and
wherein the amino acid sequence is associated with an alpha chain, the motif window includes at least 12 motif positions, and wherein identifying the start position comprises identifying the start position for the FWR3 as a one position before a 1^{st} motif position of the motif window at the candidate start position of the plurality of candidate start positions having the highest score; or
wherein the amino acid sequence is associated with a beta chain, the motif window includes five motif positions, and wherein identifying the start position comprises identifying the start position for the FWR3 as two positions before a 1^{st} motif position of the motif window at the candidate start position of the plurality of candidate start positions having the highest score.

77. The method of claim 75, wherein the amino acid sequence is associated with a heavy chain, and the motif window includes at least 10 motif positions, and wherein the evaluating comprises:
determining whether a corresponding amino acid at a 1^{st} motif position within the motif window matches one of the predetermined set of amino acids for the 1^{st} motif position, wherein the predetermined set of amino acids includes asparagine (N) and tyrosine (Y); and/or
determining whether a corresponding amino acid at a 2^{nd} motif position within the motif window is tyrosine (Y), and/or
determining whether a corresponding amino acid at a 3^{rd} motif position within the motif window matches one of the predetermined set of amino acids for the 3^{rd} motif position, wherein the predetermined set of amino acids includes alanine (A), and asparagine (N); and/or
determining whether a corresponding amino acid at a 6^{th} motif position within the motif window matches one of the predetermined set of amino acids for the 6^{th} motif position, wherein the predetermined set of amino acids includes lysine (K), glutamine (Q), and arginine (R), and/or
determining whether a corresponding amino acid at a 9^{th} motif position within the motif window matches one of the predetermined set of amino acids for the 9^{th} motif position, wherein the predetermined set of amino acids includes lysine (K), and arginine (R); and/or
determining whether a corresponding amino acid at a 10^{th} motif position within the motif window matches one of the predetermined set of amino acids for the 10^{th} motif position, wherein the predetermined set of amino acids includes alanine (A), phenylalanine (F), valine (V), and leucine (L).

78-85. (canceled)

86. The method of claim 75, wherein the amino acid sequence is associated with a light chain, the motif window includes at least 8 motif positions, and wherein the evaluating comprises:
determining whether a corresponding amino acid at a 1^{st} motif position within the motif window is glycine (G), and/or
determining whether a corresponding amino acid at a 3^{rd} motif position within the motif window is proline (P); and/or
determining whether a corresponding amino acid at a 5^{th} motif position within the motif window is arginine (R); and/or
determining whether a corresponding amino acid at a 6^{th} motif position within the motif window is phenylalanine (F); and/or
determining whether a corresponding amino acid at a 8^{th} motif position within the motif window is glycine (G).

87-92. (canceled)

93. The method of claim 75, wherein the amino acid sequence is associated with an alpha chain, the motif window includes at least 12 motif positions, and wherein the evaluating comprises:
determining whether a corresponding amino acid at a 1^{st} motif position within the motif window matches one of the predetermined set of amino acids for the 1^{st} motif position, wherein the predetermined set of amino acids includes glutamic acid (E), lysine (K), asparagine (N), and valine (V); and/or
determining whether a corresponding amino acid at a 2^{nd} motif position within the motif window matches one of the predetermined set of amino acids for the 2^{nd} motif position, wherein the predetermined set of amino acids includes alanine (A), glutamic acid (E), lysine (K), and threonine (T); and/or
determining whether a corresponding amino acid at a 3^{rd} motif position within the motif window matches one of the predetermined set of amino acids for the 3^{rd} motif position, wherein the predetermined set of amino acids includes glutamic acid (E), and serine (S); and/or
determining whether a corresponding amino acid at a 4^{th} motif position within the motif window matches one of the predetermined set of amino acids for the 4^{th} motif position, wherein the predetermined set of amino acids includes aspartic acid (D), asparagine (N), and serine (S); and/or
determining whether a corresponding amino acid at a 5^{th} motif position within the motif window is asparagine (N); and/or
determining whether a corresponding amino acid at a 6^{th} motif position within the motif window matches one of the predetermined set of amino acids for the 6^{th} motif position, wherein the predetermined set of amino acids includes glycine (G), methionine (M), and arginine (R); and/or
determining whether a corresponding amino acid at a 7^{th} motif position within the motif window matches one of the predetermined set of amino acids for the 7^{th} motif position, wherein the predetermined set of amino acids includes alanine (A), phenylalanine (F), isoleucine (I), and tyrosine (Y); and/or
determining whether a corresponding amino acid at a 8^{th} motif position within the motif window matches one of the predetermined set of amino acids for the 8^{th} motif position, wherein the predetermined set of amino acids includes serine (S) and threonine (T); and/or
determining whether a corresponding amino acid at a 9^{th} motif position within the motif window matches one of the predetermined set of amino acids for the 9^{th} motif position, wherein the predetermined set of amino acids includes alanine (A) and valine (V); and/or
determining whether a corresponding amino acid at a 10^{th} motif position within the motif window matches one of the predetermined set of amino acids for the 10^{th} motif position, wherein the predetermined set of amino acids includes glutamic acid (E) and threonine (T); and/or
determining whether a corresponding amino acid at a 12^{th} motif position within the motif window matches one of the predetermined set of amino acids for the 12^{th} motif position, wherein the predetermined set of amino acids includes aspartic acid (D) and asparagine (N).

94-105. (canceled)

106. The method of claim 75, wherein the amino acid sequence is associated with a beta chain, the motif window includes five motif positions, and wherein the evaluating comprises:
determining whether a corresponding amino acid at a 1^{st} motif position within the motif window matches one of the predetermined set of amino acids for the 1^{st} motif position, wherein the predetermined set of amino acids includes aspartic acid (D), glutamic acid (E), and lysine (K), and/or
determining whether a corresponding amino acid at a 2^{nd} motif position within the motif window matches one of the predetermined set of amino acids for the 2^{nd} motif position, wherein the predetermined set of amino acids includes glycine (G), glutamine (Q), and serine (S); and/or
determining whether a corresponding amino acid at a 3^{rd} motif position within the motif window matches one of the predetermined set of amino acids for the 3^{rd} motif position, wherein the predetermined set of amino acids includes aspartic acid (D), glutamic acid (E), glycine (G), and serine (S); and/or
determining whether a corresponding amino acid at a 4^{th} motif position within the motif window matches one of the predetermined set of amino acids for the 4^{th} motif position, wherein the predetermined set of amino acids includes isoleucine (I), leucine (L), methionine (M), and valine (V); and/or
determining whether a corresponding amino acid at a 5^{th} motif position within the motif window matches one of the predetermined set of amino acids for the 5^{th} motif position, wherein the predetermined set of amino acids includes proline (P) and serine (S).

107-110. (canceled)

111. The method of claim 1, further comprising:
determining whether the start position identified for the selected region of interest meets a set of validation criteria; and
providing an indication that the start position is not valid in response to a determination that the start position does not meet the set of validation criteria; and/or
detecting a presence of a set of indels within the amino acid sequence, and
updating the start position based on the set of indels; and/or
generating a sequence output that includes a identification of a region sequence for the selected region of interest, the start position for the selected region of interest, and a stop position for the selected region of interest, wherein the identification has an amino acid format; and/or
generating a sequence output that includes an identification of a region sequence for the selected region of interest, the start position for the selected region of interest, and a stop position for the selected region of interest, wherein the identification has a nucleotide format.

112-135. (canceled)
